(19) United States
(12) Patent Application Publication
(10) Pub. No.: US 2009/0163406 A1
(43) Pub. Date: Jun. 25, 2009

(54) COMPOSITIONS AND METHODS FOR DIAGNOSING AND TREATING BRAIN CANCER AND IDENTIFYING NEURAL STEM CELLS

(75) Inventors: Harley I. Kornblum, Los Angeles, CA (US); Daniel H. Geschwind, Santa Monica, CA (US); Ichiro Nakano, Los Angeles, CA (US); Joseph D. Dougherty, New York, NY (US); Jorge Lazareff, Los Angeles, CA (US); Paul S. Mischel, Los Angeles, CA (US); Michael D. Masterman Smith, Playa Del Rey, CA (US); Jing Huan, Los Angeles, CA (US); Houman D. Hemmati, Baltimore, MD (US)

Correspondence Address: MORRISON & FOERSTER LLP, 12531 HIGH BLUFF DRIVE, SUITE 100, SAN DIEGO, CA, 92130-2040, US

(73) Assignee: THE REGENTS OF THE UNIVERSITY OF CALIFORNIA, Oakland, CA (US)

(21) Appl. No.: 11/576,444

(22) PCT Filed: Sep. 30, 2005
(86) PCT No.: PCT/US05/35355

§ 371 Date: Jun. 5, 2008

Related U.S. Application Data
(60) Provisional application No. 60/614,511, filed on Sep. 30, 2004.
(60) Provisional application No. 60/671,696, filed on Apr. 15, 2005.

(51) Int. Cl. A61K 31/70; A61K 31/7088; A61K 38/02; A61K 38/16; A61K 31/20; C40B 40/08; G01N 33/53; A61P 35/00

(52) U.S. Cl. 514/12; 514/44; 514/2; 514/23; 514/558; 506/17; 435/7.21

(57) ABSTRACT

In one aspect, the invention provides composition and methods for the diagnosis, prognosis and treatment of tumors and cancers, e.g., brain cancers. In one aspect, the invention provides compositions and methods for inhibiting the growth, proliferation, differentiation and/or survival of a neural stem cell or a cancer cell, or progenitor stem cell thereof. In one aspect, the invention provides compositions and methods for identifying the genetic profile of a brain cancer cell or a self-renewing neural cancer stem cell. In one aspect, the invention provides methods employing these profiles to identify compounds that inhibit tumor growth.

### STATEMENT OF RIGHTS TO INVENTIONS MADE UNDER FEDERALLY SPONSORED RESEARCH

This invention is supported in part by Grant No. MH65756 of the National Institutes of Health. The United States government may have certain rights in this invention.

### TECHNICAL FIELD

The present invention relates to the fields of cancer, neurology and medicine. In one aspect, the invention provides compositions and methods for the diagnosis, prognosis and treatment of tumors and cancers, e.g., brain cancers. In one aspect, the invention provides compositions and methods for inhibiting the growth, proliferation, differentiation and/or survival of a neural stem cell or a cancer cell, or progenitor stem cell thereof. In one aspect, the invention provides compositions and methods for identifying the genetic profile of a brain cancer cell or a self-renewing neural cancer stem cell. In one aspect, the invention provides methods employing these profiles to identify compounds that inhibit tumor growth.

### BACKGROUND

Brain tumors frequently occur in children and represent the leading cause of cancer mortality in this population. However, still little is known regarding the cellular and genetic makeup of the neural tumor cell. Recent evidence suggests that pediatric brain tumors develop from cells that have many of the same characteristics as neural stem cells. See, e.g., Hemmati (2003) Proc. Natl. Acad. Sci. USA 100:15175-15183.

Self-renewal and multipotency are critical properties of stem cells. This is certainly the case with neural stem cells which are defined by their ability to self-renew, and their capacity to produce the three major cell types of the brain: neurons, astrocytes and oligodendrocytes. In the adult subventricular zone (SVZ), type B cells, a slowly dividing glial fibrillary acidic protein (GFAP)-positive cell type, are thought to be neural stem cells; while type C cells, a more rapidly proliferative population of self-renewing multipotent progenitors, are derived from the type B cells. In early brain development, it is not clear whether such distinctions exist.

Multipotent progenitor cells (“MPC”) are cells that are derived from the central nervous system (CNS), self-renewing and tripotent. Genes that regulate MPC self-renewal play important roles in brain development, regulating cell number and brain size. Although cell fate determination and cell cycle regulation are thought to underlie the process of self-renewal, little is known about specific genetic mechanisms that regulate this process. Identification of specific genetic mechanisms will provide critical insights for development biology as well as provide improved diagnostic tests and therapeutic targets.

A genome-wide screening strategy has been used to discover genes that regulate MPC function. It was reasoned that genes expressed by neural stem/progenitor cell populations and not differentiated cells would be those involved in self-renewal, a fundamental feature of MPC. To identify such genes, a custom, subtracted cDNA microarray was used to discover genes expressed in multiple NSC-containing neurospheres. A screening in situ hybridization analysis was used to narrow this pool of genes by determining which ones were highly expressed in developing germinal zones in vivo. This process identified numerous genes that are enriched in neural progenitors. Many of these genes were expressed within CNS germinal zones in vivo, and thus were candidates for playing roles in MPC function.

MELK, also known as MPK38 was found to be present in multiple NSC-containing populations and in hematopoietic stem cells. MELK is a member of the snf1/AMPK family of kinases. Although several members of the family are known to play roles in cell survival under metabolically challenging conditions, the function of MELK has not previously been determined.

### SUMMARY

The invention provides compositions, e.g., pharmaceutical compositions, for inhibiting the growth (proliferation), differentiation or survival of a neural stem cell or a cancer cell, comprising at least one compound capable of (a) inhibiting transcription of a gene or inhibiting translation of a gene's transcript, wherein the gene is selected from the group consisting of a maternal embryonic leucine zipper kinase (MELK) gene, a T-LAK cell-originated protein kinase (TOPK) gene, a phosphoserine phosphatase (PSP) gene, a forkhead box M1 (FoxM1) gene, a B-myb gene, a Rho/Rac/Cdc42-like GTPase activating protein (RACGAP) gene, a kinesin superfamily protein member 4 (KIF4) or KIF4A gene, a cell cycle control protein CDC2 gene, a EZHa gene, a HCAP-G gene (HCAP-G is a structural maintenance of chromosome (SMC) family protein), a minichromosome maintenance (MCM) protein 7 (MCM7) gene, a chromatin assembly factor-1A (CHAF-1A) gene, a minichromosome maintenance protein 6 (MCM6) gene, a thymopoietin (TMPO) gene, a sperm associated antigen 5 (SPAG5) gene (see Shao (2001) Mol. Reprod. Dev. 59:410-416), a baculoviral IAP repeat-containing 5 (BIRC5) gene, a thymidylate synthase (TYMS) gene, a karyopherin (importin) alpha 2 (KPNA2) gene, a kinesin family member 2C (KIF2c) gene, a MAD2 (mitotic arrest deficient, homolog)-like 1 (MAD2L1) gene, a NIMA (never in mitosis gene a)-related kinase 2 (NEK2) gene, a BUB1 budding uninhibited by benzimidazoles 1 homolog beta (yeast) (BUB1B) gene, a epithelial cell transforming sequence 2 oncogene (ECT2) gene, a ubiquitin-conjugating enzyme E2C (UBE2C) gene, a fatty acid elongase (FEN1) gene, a H2A histone family, member X (H2AFX) gene, a serine/threonine kinase 6 (STK6) gene, a methyltransferase T1 (DNMT1) gene, a proliferating cell nuclear antigen (PCNA) gene, a polymerase A (POLA) gene, a thyroid hormone receptor interactor 13 (TRIP13) gene, a monoclonal antibody Ki-67 (MKI67) gene (or, a monoclonal antibody Ki-67 (MKI67) gene) and a solute carrier family 35, member B1 (SLC35B1) gene (“exemplary genes”); or (b) inhibiting the expression or activity of protein selected from the group consisting of a maternal embryonic leucine zipper kinase (MELK) protein, a T-LAK cell-originated protein kinase (TOPK), a phosphoserine phosphatase (PSP), a forkhead box M1 (FoxM1) protein, a B-myb protein, a Rho/Rac/Cdc42-like GTPase activating protein (RACGAP), a kinesin superfamily protein member 4 (KIF4) or KIF4A protein, a cell cycle control protein CDC2, a EZHa protein, a HCAP-G protein, a MCM7 protein, a CHAF1A protein, a MCM6 protein, a TMPO protein, a SPAG5 protein, a BIRC5 protein, a TYMS protein, a KPNA2 protein, a KIF2c protein, a MAD2L1 protein, a NEK2 protein, a BUB1B protein, a ECT2 protein, a UBE2C protein, a FEN1 protein, a H2AFX protein, a STK6 protein, a DNMT1 protein, a PCNA protein, a POLA protein, a TRIP13 protein, a MK167 protein and a solute carrier family 35 (SLC35B1) protein. The invention provides pharmaceutical compositions comprising at least one of these compositions, and a pharmaceutically acceptable excipient.

In one aspect, the compositions, e.g., pharmaceutical compositions of the invention, inhibit the growth, differentiation or survival of a neural stem cell or a cancer cell by inhibiting the expression of a gene or gene message or protein product that contributes to the growth, differentiation or survival of the neural stem cell or a cancer cell. In one aspect, if the composition is to be used in vivo, the compositions are pharmaceutical compositions comprising a pharmaceutically acceptable excipient, e.g., the pharmaceutical compositions of the invention can be formulated in any acceptable and appropriate manner, depending on whether they comprise nucleic acids, proteins or a combination thereof. In one aspect, if the composition is to be used in vitro, the compositions are formulated for the appropriate use, e.g., in cell or tissue culture.

In one aspect, the composition of the invention or the pharmaceutical composition of the invention comprises at least two, three, four or five or more compounds capable of inhibiting the growth, proliferation, differentiation and/or survival of a neural stem cell or a cancer cell.

In one aspect, the at least one compound (in a composition of the invention or the pharmaceutical composition of the invention) inhibits the growth, proliferation, differentiation and/or survival of a brain tumor cell or a stem cell progenitor thereof. The at least one compound can inhibit growth, proliferation, differentiation and/or survival of a granule cell precursor cell or a self-renewing neural cancer cell or a stem cell progenitor thereof. The at least one compound can comprise a nucleic acid, a carbohydrate, a fat, a small molecule or a polypeptide or peptide. In one aspect, the at least one nucleic acid compound capable of inhibiting transcription of a gene or inhibiting translation of a gene's transcript nucleic acid comprises an oligonucleotide, e.g., the oligonucleotide can comprise an antisense oligonucleotide, a ribozyme, a double-stranded inhibitory RNA (RNAi) molecule, an RNase III-prepared short interfering RNA (esiRNA) or a vector-derived short hairpin RNAs (shRNA). In one aspect, the antisense oligo, ribozyme, double-stranded inhibitory RNA (RNAi) molecule, RNase III-prepared short interfering RNA (esiRNA) or vector-derived short hairpin RNAs (shRNA) comprises a subsequence of a transcriptional activation sequence (e.g., a promoter or enhancer sequence) or a message of a maternal embryonic leucine zipper kinase (MELK) gene, a T-LAK cell-originated protein kinase (TOPK) gene, a phosphoserine phosphatase (PSP) gene, a forkhead box M1 (FoxM1) gene, a B-myb gene, a Rho/Rac/Cdc42-like GTPase activating protein (RACGAP) gene, a kinesin superfamily protein member 4 (KIF4) or KIF4A gene, a cell cycle control protein CDC2 gene, a EZHa gene, a HCAP-G gene, a MCM7 gene, a CHAF1A gene, a MCM6 gene, a TMPO gene, a SPAG5 gene, a BIRC5 gene, a TYMS gene, a KPNA2 gene, a KIF2c gene, a MAD2L1 gene, a NEK2 gene, a BUB1B gene, a ECT2 gene, a UBE2C gene, a FEN1 gene, a H2AFX gene, a STK6 gene, a DNMT1 gene, a PCNA gene, a POLA gene, a TRIP13 gene, a MK167 (proliferation-related Ki-67 antigen) gene or a solute carrier family 35 (SLC35B1) gene.

In one aspect, the at least one polypeptide or peptide compound (in a composition of the invention or the pharmaceutical composition of the invention) is capable of inhibiting transcription of a gene or inhibiting translation of a gene's transcript nucleic acid comprises (a) an antibody, or (b) a polypeptide or peptide capable of binding a transcriptional activator of a maternal embryonic leucine zipper kinase (MELK) gene, a T-LAK cell-originated protein kinase (TOPK) gene, a phosphoserine phosphatase (PSP) gene, a forkhead box M1 (FoxM1) gene, a B-myb gene, a Rho/Rac/Cdc42-like GTPase activating protein (RACGAP) gene, a kinesin superfamily protein member 4 (KIF4) or KIF4A gene, a cell cycle control protein CDC2 gene, a EZHa gene, a HCAP-G gene, a MCM7 gene, a CHAF1A gene, a MCM6 gene, a TMPO gene, a SPAG5 gene, a BIRC5 gene, a TYMS gene, a KPNA2 gene, a KIF2c gene, a MAD2 μl gene, a NEK2 gene, a BUB1B gene, a ECT2 gene, a UBE2C gene, a FEN1 gene, a H2AFX gene, a STK6 gene, a DNMT1 gene, a PCNA gene, a POLA gene, a TRIP13 gene, a MK167 (proliferation-related Ki-67 antigen) gene or a solute carrier family 35 (SLC35B1) gene.

The invention provides methods for inhibiting the growth, proliferation, differentiation and/or survival of a neural stem cell or a cancer cell or progenitor stem cell thereof, comprising the steps of contacting the cell with a composition of the invention (e.g., compositions for inhibiting the growth, proliferation, differentiation and/or survival of a neural stem cell or a cancer cell, or progenitor stem cell thereof, as described herein). In one aspect, the neural stem cell or a cancer cell is a neural tumor cell proliferation or a progenitor thereof.

The invention provides methods for inhibiting the growth, proliferation, differentiation and/or survival of a neural stem cell or a cancer cell, or progenitor stem cell thereof, in an individual in need thereof, comprising the steps of administering to the individual a therapeutically effective amount of a pharmaceutical composition of the invention (e.g., pharmaceutical compositions for inhibiting the growth, proliferation, differentiation and/or survival of a neural stem cell or a cancer cell, or progenitor stem cell thereof, as described herein).

The invention provides arrays (e.g., microarrays) comprising (a) at least one nucleic acid comprising a gene sequence or a transcript or cDNA sequence, wherein the sequence comprises a maternal embryonic leucine zipper kinase (MELK) sequence, a T-LAK cell-originated protein kinase (TOPK) sequence, a phosphoserine phosphatase (PSP) sequence, a forkhead box M1 (FoxM1) sequence, a B-myb sequence, a Rho/Rac/Cdc42-like GTPase activating protein (RACGAP) sequence, a kinesin superfamily protein member 4 (KIF4) or KIF4A sequence, a cell cycle control protein CDC2 sequence, a EZHa sequence, a HCAP-G sequence, a MCM7 sequence, a CHAF1A sequence, a MCM6 sequence, a TMPO sequence, a SPAG5 sequence, a BIRC5 sequence, a TYMS sequence, a KPNA2 sequence, a KIF2c sequence, a MAD2L1 sequence, a NEK2 sequence, a BUB1B sequence, a ECT2 sequence, a UBE2C sequence, a FEN1 sequence, a H2AFX sequence, a STK6 sequence, a DNMT1 sequence, a PCNA sequence, a POLA sequence, a TRIP13 sequence, a MK167 (proliferation-related Ki-67 antigen) sequence or a solute carrier family 35 (SLC35B1) sequence, or a combination thereof; or (b) at least one protein or peptide comprising a sequence or subsequence of a protein or peptide comprising a maternal embryonic leucine zipper kinase (MELK) protein, a T-LAK cell-originated protein kinase (TOPK), a phosphoserine phosphatase (PSP), a forkhead box M1 (FoxM1) protein, a B-myb protein, a Rho/Rac/Cdc42-like GTPase activating protein (RACGAP), a kinesin superfamily protein member 4 (KIF4) or KIF4A protein, a cell cycle control protein CDC2, a EZHa protein, a HCAP-G protein, a MCM7 protein, a CHAF1A protein, a MCM6 protein, a TMPO protein, a SPAG5 protein, a BIRC5 protein, a TYMS protein, a KPNA2 protein, a KIF2c protein, a MAD2L1 protein, a NEK2 protein, a BUB1B protein, a ECT2 protein, a UBE2C protein, a FEN1 protein, a H2AFX protein, a STK6 protein, a DNMT1 protein, a-PCNA protein, a POLA protein, a TRIP13 protein, a MK167 (proliferation-related Ki-67 antigen) protein or a solute carrier family 35 (SLC35B1) protein, or a combination thereof.

The invention provides one or more compilation(s) of probes comprising (a) at least two nucleic acids comprising a gene sequence or a transcript or cDNA sequence, wherein the sequence comprises a maternal embryonic leucine zipper kinase (MELK) sequence, a T-LAK cell-originated protein kinase (TOPK) sequence, a phosphoserine phosphatase (PSP) sequence, a forkhead box M1 (FoxM1) sequence, a B-myb sequence, a Rho/Rac/Cdc42-like GTPase activating protein (RACGAP) sequence, a kinesin superfamily protein member 4 (KIF4) or KIF4A sequence, a cell cycle control protein CDC2 sequence, a EZHa sequence, a HCAP-G sequence, a MCM7 sequence, a CHAF1A sequence, a MCM6 sequence, a TMPO sequence, a SPAG5 sequence, a BIRC5 sequence, a TYMS sequence, a KPNA2 sequence, a KIF2c sequence, a MAD2L1 sequence, a NEK2 sequence, a BUB1B sequence, a ECT2 sequence, a UBE2C sequence, a FEN1 sequence, a H2AFX sequence, a STK6 sequence, a DNMT1 sequence, a PCNA sequence, a POLA sequence, a TRIP13 sequence, a MK167 (proliferation-related Ki-67 antigen) sequence or a solute carrier family 35 (SLC35B1) sequence, or a combination thereof; or (b) at least two proteins or peptides capable of binding specifically to a protein comprising a sequence or subsequence of a maternal embryonic leucine zipper kinase (MELK) protein, a T-LAK cell-originated protein kinase (TOPK), a phosphoserine phosphatase (PSP), a forkhead box M1 (FoxM1) protein, a B-myb protein, a Rho/Rac/Cdc42-like GTPase activating protein (RACGAP), a kinesin superfamily protein member 4 (KIF4) or KIF4A protein, a cell cycle control protein CDC2, a EZHa protein, a HCAP-G protein, a MCM7 protein, a CHAF1A protein, a MCM6 protein, a TMPO protein, a SPAG5 protein, a BIRC5 protein, a TYMS protein, a KPNA2 protein, a KIF2c protein, a MAD2L1 protein, a NEK2 protein, a BUB1B protein, a ECT2 protein, a UBE2C protein, a FEN1 protein, a H2AFX protein, a STK6 protein, a DNMT1 protein, a PCNA protein, a POLA protein, a TRIP13 protein, a MK167 (proliferation-related Ki-67 antigen) protein or a solute carrier family 35 (SLC35B1) protein, or a combination thereof.

The invention provides methods of identifying a compound that inhibits the growth, growth, proliferation, differentiation or survival differentiation or survival of a neural stem cell or a cancer or tumor cell, or a progenitor stem cell thereof, comprising (a) providing a candidate compound and a neural stem cell, a cancer or tumor cell, or a progenitor stem cell thereof; (b) contacting the cell with a candidate compound; (c) measuring the level of expression of at least one of a maternal embryonic leucine zipper kinase (MELK) gene, a T-LAK cell-originated protein kinase (TOPK) gene, a phosphoserine phosphatase (PSP) gene, a forkhead box M1 (FoxM1) gene, a B-myb gene, a Rho/Rac/Cdc42-like GTPase activating protein (RACGAP) gene, a kinesin superfamily protein member 4 (KIF4) or KIF4A gene, a cell cycle control protein CDC2 gene, a EZHa gene, a HCAP-G gene, a MCM7 gene, a CHAF1A gene, a MCM6 gene, a TMPO gene, a SPAG5 gene, a BIRC5 gene, a TYMS gene, a KPNA2 gene, a KIF2c gene, a MAD2L1 gene, a NEK2 gene, a BUB1B gene, a ECT2 gene, a UBE2C gene, a FEN1 gene, a H2AFX gene, a STK6 gene, a DNMT1 gene, a PCNA gene, a POLA gene, a TRIP13 gene, a MK167 (proliferation-related Ki-67 antigen) gene or a SLC35B1 gene, or a combination thereof, wherein the level of expression of the gene is measured by determining the level of expression of the gene, a message transcribed by the gene or a protein encoded by the gene; and (d) comparing under substantially the same conditions the level of expression of at least one of the gene, message or protein in a cell not contacted by the candidate compound to the level of expression of at least one of the gene, message or protein in a cell contacted by the compound, whereby the candidate compound is identified as a compound that inhibits the growth, proliferation, differentiation or survival of the cell growth as one that decreases expression the gene, message or protein. In one aspect, this method further comprising assessing the inhibition of growth, proliferation, differentiation, survival and/or self-renewal potential of the cell in the presence of the compound. In one aspect, the growth, proliferation, differentiation and/or survival inhibition is assessed by primary sphere formation assay, proliferation or differentiation potential. In one aspect, the compound is identified as an inhibitor of growth or proliferation when proliferation or growth of the cell in the presence of the compound is 5%, 10%, 20%, 25%, 30%, 40%, 50%, 55%, 60%, 65%, 70%, 75%, 80%, 85%, 90% or more inhibited in the presence of the compound.

The invention provides methods of identifying a candidate compound for inhibiting growth or proliferation of a neural stem cell or a cancer or tumor cell, or a progenitor stem cell thereof, comprising (a) providing a candidate compound; (b) contacting the candidate compound with a protein comprising a sequence or subsequence of a maternal embryonic leucine zipper kinase (MELK) protein, a T-LAK cell-originated protein kinase (TOPK), a phosphoserine phosphatase (PSP), a forkhead box M1 (FoxM1) protein, a B-myb protein, a Rho/Rac/Cdc42-like GTPase activating protein (RACGAP), a kinesin superfamily protein member 4 (KIF4) or KIF4A protein, a cell cycle control protein CDC2, a EZHa protein, a HCAP-G protein, a MCM7 protein, a CHAF1A protein, a MCM6 protein, a TMPO protein, a SPAG5 protein, a BIRC5-protein, a TYMS protein, a KPNA2 protein, a KIF2c protein, a MAD2L1 protein, a NEK2 protein, a BUB1B protein, a ECT2 protein, a UBE2C protein, a FEN1 protein, a H2AFX protein, a STK6 protein, a DNMT1 protein, a PCNA protein, a POLA protein, a TRIP13 protein, a MK167 (proliferation-related Ki-67 antigen) protein or a solute carrier family 35 (SLC35B1) protein, or a combination thereof; and (c) measuring or determining the effect of the compound on the biological activity of the protein, whereby a compound that inhibits at least one biological activity of at least one protein is identified as a candidate compound for inhibiting growth or proliferation of a neural stem cell or a cancer or tumor cell, or a progenitor stem cell thereof. In one aspect, inhibition of at least one biological activity of at least one protein identifies the compound as a candidate compound for inhibiting the growth, proliferation, differentiation and/or survival of a granule cell precursor cell or a self-renewing neural cancer cell or a stem cell progenitor thereof.

The invention provides methods of diagnosing the metastatic potential of a tumor, e.g., a CNS or brain tumor, such as a neural tumor, comprising determining the presence or absence of expression of a maternal embryonic leucine zipper kinase (MELK) protein, a T-LAK cell-originated protein kinase (TOPK), a phosphoserine phosphatase (PSP), a forkhead box M1 (FoxM1) protein, a B-myb protein, a Rho/Rac/Cdc42-like GTPase activating protein (RACGAP), a kinesin superfamily protein member 4 (KIF4) or KIF4A protein, a cell cycle control protein CDC2, a EZHa protein, a HCAP-G protein, a MCM7 protein, a CHAF1A protein, a MCM6 protein, a TMPO protein, a SPAG5 protein, a BIRC5 protein, a TYMS protein, a KPNA2 protein, a KIF2c protein, a MAD2L1 protein, a NEK2 protein, a BUB1B protein, a ECT2 protein, a UBE2C protein, a FEN1 protein, a H2AFX protein, a STK6 protein, a DNMT1 protein, a PCNA protein, a POLA protein, a TRIP13 protein, a MK167 (proliferation-related Ki-67 antigen) protein or a solute carrier family 35 (SLC35B1) protein, or a combination thereof.

In one aspect, the invention provides compositions and methods for identifying the genetic profile of a brain cancer cell or a self-renewing neural cancer stem cell. For example, the invention provides compositions and methods for inhibiting MELK, T-LAK cell-originated protein kinase (TOPK), phosphoserine phosphatase (PSP), forkhead box M1 (FoxM1), and/or B-myb expression as a means treating metastatic neural tumors. The invention also provides compositions and methods for detecting MELK, T-LAK cell-originated protein kinase (TOPK), phosphoserine phosphatase (PSP), forkhead box M1 (FoxM1), and/or B-myb expression as a means of diagnosing or predicting the onset of metastatic neural tumors. In one aspect, the invention provides compositions and methods using the profiles of these genes to access tumor types, the aggressiveness of tumor growth, to correlate particular treatment successes with particular gene expression profiles, thus aiding the clinician in the selection of a particular treatment plan and helping access the chances of success of any particular treatment plan.

In one aspect, the invention provides methods employing these profiles to identify compounds that inhibit tumor growth. In the aspect, the compositions and methods of the invention are used to identify the genetic profile of a cancer cell or a stem cell, e.g., a neural cancer stem cell or a neural cancer cell, or any progenitor thereof of either, and the use of this profile to identify compounds that modulate cancer cell or a stem cell, e.g., neural cancer stem cell, survival, growth and/or differentiation. Additionally, the genetic profiles provided herein provide a useful diagnostic tool for neural tumors, particularly pediatric tumors.

In one aspect, the invention provides an isolated neural cancer stem cell having enriched expression of maternal embryonic leucine zipper kinase (MELK) gene. The cell can further comprising enriched expression of one or more of the following known genes or their encoded proteins, including T-LAK cell-originated protein kinase (TOPK), phosphoserine phosphatase (PSP), forkhead box M1 (FoxM1), B-myb, Rho/Rac/Cdc42-like GTPase activating protein (RACGAP), kinesin superfamily protein member 4 (KIF4) or KIF4A, cell cycle control protein CDC2, EZHa, HCAP-G, MCM7, CHAF1A, MCM6, TMPO, SPAG5, BIRC5, TYMS, KPNA2, KIF2c, MAD2 μl, NEK2, BUB1B, ECT2, UBE2C, FEN1, H2AFX, STK6, DNMT1, PCNA, POLA, TRIP13, MK167 (proliferation-related Ki-67 antigen), and/or solute carrier family 35 (SLC35B1).

Also provided herein is a method of using the gene profile of the neural cancer stem cell or cancer cell to identify compounds useful in inhibiting their growth or survival, e.g., for use in inhibiting brain tumor growth. Thus, provided herein is a method of identifying a compound useful in inhibiting their growth or survival, e.g., for use in inhibiting tumor, e.g., brain tumor, growth comprising (a) contacting the cell (e.g., neural cancer stem cell or cancer cell) with a candidate compound; (b) assessing the level of expression of maternal embryonic leucine zipper kinase (MELK), T-LAK cell-originated protein kinase (TOPK), phosphoserine phosphatase (PSP), forkhead box M1 (FoxM1), B-myb, RACGAP1, KIF4A, CDC2, EZHa, HCAP-G, MCM7, CHAF1A, MCM6, TMPO, SPAG5, BIRC5, TYMS, KPNA2, KIF2c, MAD2 μl, NEK2, BUB1B, ECT2, UBE2C, FEN1, H2AFX, STK6, DNMT1, PCNA, POLA, TRIP13, MK167 (proliferation-related Ki-67 antigen), and/or SLC35B1; and (c) comparing the level of expression with a cell in the absence of the candidate compound, whereby the candidate compound is identified as a compound that inhibits tumor growth as one that modulates expression levels resulting in an inhibition of tumor growth. The candidate compound can be a protein, a nucleic acid, a carbohydrate (e.g., a polysaccharide), a fat or a small molecule or a combination thereof.

Cell growth inhibition or inhibition of cell survival (e.g., diminishing cell vitality, or decreasing the survivability of a cell, or increasing its sensitivity to apoptosis signals, etc.), including tumor growth inhibition, can be assessed by protocols comprising measuring cell proliferation, cell differentiation capacity or cell self-renewal potential, or a combination thereof. Exemplary assays comprise primary sphere formation assay, proliferation and differentiation potential.

In one embodiment, the candidate compound is identified as an inhibitor of growth or proliferation of a cell (e.g., as a tumor growth inhibitor) when proliferation of the cell (e.g., a stem cell or cancer cell) in the presence of the compound is at least 10%, 20%, 30%, 40%, 50%, 55%, 60%, 65%, 70%, 75%, 80%, 85%, 90% or less of the proliferation of said stem cell in the absence of said compound. Alternatively, the candidate compound is identified as an inhibitor of growth or proliferation of a cell (e.g., as a tumor growth inhibitor) when proliferation of the cell (e.g., a stem cell or cancer cell) in the presence of the compound is inhibited by at least 10%, 20%, 30%, 40%, 50%, 55%, 60%, 65%, 70%, 75%, 80%, 85%, 90% or more in the presence of the compound (i.e., after the cell have been contacted by the compound).

Further provided is a method of identifying a compound that inhibits growth or proliferation of a cell (e.g., as a tumor growth inhibitor), comprising (a) contacting the protein of maternal embryonic leucine zipper kinase (MELK), T-LAK cell-originated protein kinase (TOPK), phosphoserine phosphatase (PSP), forkhead box M1 (FoxM1), B-myb, RACGAP1, KIF4A, CDC2, EZHa, HCAP-G, MCM7, CHAF1A, MCM6, TMPO, SPAG5, BIRC5, TYMS, KPNA2, KIF2c, MAD2L1, NEK2, BUB1B, ECT2, UBE2C, FEN1, H2AFX, STK6, DNMT1, PCNA, POLA, TRIP13, MK167 (proliferation-related Ki-67 antigen), and/or SLC35B1; and (c) assessing the effect of the compound on the biological activity of said protein, whereby an inhibitor is identified as the compound that reduces at least one biological activity of said protein.

Also provided herein is a method for inhibiting growth or proliferation of a cell (e.g., as a tumor growth inhibitor, an inhibitor of proliferation of a neural tumor cell), comprising administering a compound identified by the inhibition of expression of maternal embryonic leucine zipper kinase (MELK), T-LAK cell-originated protein kinase (TOPK), phosphoserine phosphatase (PSP), forkhead box M1 (FoxM1), B-myb, RACGAP1, KIF4A, CDC2, EZHa, HCAP-G, MCM7, CHAF1A, MCM6, TMPO, SPAG5, BIRC5, TYMS, KPNA2, KIF2c, MAD2 μl, NEK2, BUB1B, ECT2, UBE2C, FEN1, H2AFX, STK6, DNMT1, PCNA, POLA, TRIP13, MK167 (proliferation-related Ki-67 antigen), and/or SLC35B1, or the inhibition of the activity of the protein encoded by these genes. In some embodiments, the inhibitor is an oligonucleotide, e.g., an antisense oligonucleotide, a ribozyme, a double-stranded inhibitory RNA (RNAi) molecule, an RNase 111-prepared short interfering RNA (esiRNA) or a vector-derived short hairpin RNAs (shRNA).

Further, provided herein are methods of diagnosing the metastatic potential of a neural tumor comprising determining the presence or absence of MELK expression, or the presence or absence of TOPK.

Also provided herein are kits comprising at least one composition of the invention (e.g., nucleic acids and/or proteins for inhibiting the growth, proliferation, differentiation and/or survival of a neural stem cell or a cancer cell, or progenitor stem cell thereof); and, in one aspect, instructions for practicing the methods provided herein.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIGS. 1A, 1B and 1C illustrate data that demonstrates that both MELK and B-myb are highly expressed in proliferating granule cell precursors in the neonatal brains, as described in detail in Example 1, below.

FIGS. 2A, 2B and 2C illustrate data showing the role of MELK in medulloblastomas, where MELK expression was determined by in situ hybridization using a cerebellum bearing a spontaneous tumor, as described in detail in Example 1, below.

FIG. 3 (FIG. 3A) illustrates data showing that MELK is highly expressed in human medulloblastoma, as described in detail in Example 1, below.

FIG. 4Aa, FIG. 4Ab, FIG. 4Ba, FIG. 4Bb, FIG. 4Bc and FIG. 4Bd, illustrate data showing RNAi treatment targeting MELK inhibits human medulloblastoma growth in vitro, as described in detail in Example 1, below.

FIGS. 5A and 5B illustrate data showing the signaling of MELK is not dependent on sonic hedgehog or akt-mTOR, as described in detail in Example 1, below.

FIGS. 6A, 6B, 6C, 6D and 6F illustrate data showing PBK/TOPK mRNA is specifically expressed in all germinal zones throughout neural development, as described in detail in Example 5, below.

FIGS. 7A, 7B and 7C illustrate data showing that PBK/TOPK protein structure and expression in tumors suggests role in late cell cycle, as described in detail in Example 5, below.

FIGS. 8A, 8B, 8C and 8D illustrate data showing phospho-PBK/TOPK expression is only detected during mitosis, as described in detail in Example 5, below.

FIG. 9A through 9I illustrate data showing that PBK/TOPK is expressed by proliferating progenitors in vitro, and its activity is required for normal cell cycle, as described in detail in Example 5, below.

FIGS. 10A, 10B, 10C, 10D and 10E illustrate data showing PBK/TOPK protein is not expressed in neurons or mature glia in EGL or the SEZ and RMS, as described in detail in Example 5, below.

FIGS. 11A, 11B, 11C, 11D and 11E illustrate data showing PBK/TOPK expressed exclusively in rapidly proliferating progenitor cells in postnatal rodent brain, as described in detail in Example 5, below.

FIGS. 12A and 12B illustrate data showing PBK/TOPK cells were dramatically reduced when stem cells are ablated, as described in detail in Example 5, below.

FIGS. 13A, 13B and 13C illustrate data showing PBK/TOPK cells are GFAP negative progeny of GFAP positive cells, as described in detail in Example 5, below.

FIG. 14 shows a model of PBK/TOPK expression in adult neurogenesis, as described in detail in Example 5, below.

FIG. 15A lists gene-specific primers used to identify genes used to practice the invention, including genes whose expression is inhibited to inhibit the growth, proliferation, differentiation and/or survival of a neural stem cell or a cancer cell, or progenitor stem cell thereof, or treat a cancer or tumor cell, as explained in detail in Examples 1 and 7, below.

FIG. 16 illustrates data from an RT-PCR analysis of brain tumor and normal brain samples, as explained in detail in Example 6, below.

FIG. 17 illustrates data showing normalized MELK expression levels in a variety of tumor types and normal brain, as explained in detail in Example 6, below.

FIG. 18 illustrates a survival curve of patients with GBM divided into two groups; high vs. lower MELK expression, as explained in detail in Example 6, below.

FIGS. 19A, 19B, 19C and 19D illustrate data showing that the exemplary MELK siRNA dramatically inhibited the growth of medulloblastomas in vivo, as explained in detail in Example 6, below.

FIG. 20 illustrates data showing that the exemplary MELK siRNA inhibited sphere production in gliomas, as explained in detail in Example 6, below.

FIG. 21 illustrates data showing gene ontology of MELK correlated genes (left) and anti-correlated genes (right), as explained in detail in Example 6, below.

FIG. 22 illustrates data showing gene expression in Daoy cells treated with either MELK or Luciferase (ctrl) siRNA, as explained in detail in Example 6, below.

FIG. 23 illustrates data showing that FoXM1 and CDC25A are capable of at least partial rescue of the reduced cell number seen in MELK siRNA-treated cells, as explained in detail in Example 6, below.

FIG. 24 illustrates data demonstrating that MELK siRNA treatment ex vivo inhibits in vivo growth of ependymoma progenitors, as explained in detail in Example 6, below.

FIG. 25 illustrates data demonstrating MELK is highly enriched, in multiple neural stem cell-containing cultures. FIG. 25A illustrates data demonstrating that MELK was expressed by NS populations and downregulated after mitogen withdrawal; FIG. 25B illustrates data demonstrating MELK mRNA levels declined after bFGF withdrawal in neural progenitors; FIG. 25C illustrates data demonstrating the characteristics of NS cultures under various differentiation conditions; FIG. 25D illustrates data demonstrating the association of MELK with neural progenitors; as explained in detail in Example 7, below.

FIG. 26 illustrates data showing MELK is downregulated during ontogeny, and brain expression is restricted in the neurogenic regions throughout development. FIG. 26A illustrates data demonstrating that MELK mRNA was expressed in the developing brain during early and mid-embryonic periods; FIG. 26B illustrates data demonstrating nearly exclusive expression of MELK in CNS germinal zones at multiple ages; FIG. 26C illustrates data showing in situ hybridization of an adult section counterstained for GFAP immunoreactivity, and lack of MELK expression in HC, and presence in SVZ; as explained in detail in Example 7, below.

FIG. 27 illustrates data demonstrating that MELK is expressed only in proliferating PCNA-positive cells, but not in TuJ1-positive neuroblasts in developing brains. FIG. 27A illustrates data demonstrating MELK labeling occurred in cells expressing the proliferation marker PCNA; FIG. 27B illustrates data demonstrating that in the adult SVZ, MELK expression was detectable in GFAP-positive cells; FIG. 27C illustrates data demonstrating that MELK was expressed in the hippocampus early postnatal ages; FIG. 27D illustrates data demonstrating that MELK mRNA was identified within the external granule cell layer of the cerebellum; as explained in detail in Example 7, below.

FIG. 28 illustrates data showing that the regulatory element of MELK transcripts is localized in the upstream of its first exon, and is active only in undifferentiated neural progenitors. FIG. 28A is a figure illustrating that mouse and human MELK genes have 16 axons with a translation initiation site at exon 2; FIG. 28B illustrates data showing RT-PCR analysis to detect MELK expression both in EGFP positive and in negative populations; FIG. 28C illustrates data characterizing the cellular specificity of MELK expression in cortical progenitors derived from E12 embryos, as explained in detail in Example 7, below.

FIG. 29 lists and summarizes multiple transcription factor binding sequences in neural gene sequences, as explained in detail in Examples 1 and 7, below.

FIG. 30 illustrates data showing MELK-expressing progenitors are neurosphere (NS)-initiating stem cells. FIG. 30A illustrates data showing MELK-positive E15 progenitors generated more primary neurospheres than LeX-positive cells; FIG. 30B illustrates data showing neurospheres formed from MELK-expressing cells are derived from multipotent progenitors; as explained in detail in Example 7, below.

FIG. 31 illustrates data showing control cultures transfected with PCMV-EGFP yielded equivalent percentages of neurospheres in EGFP positive and negative fractions, as explained in detail in Example 7, below.

FIG. 32 illustrates data showing the results of manipulation of MELK influences neural progenitor proliferation—MELK-overexpressing progenitors generate more neurospheres, and MELK downregulation diminishes neurosphere numbers; and FIG. 32A shows the experimental strategy employed; FIG. 32B illustrates data showing the characterization of adherent progenitors from neurospheres generated from E12 telencephalon and P0 cerebral cortices; FIG. 32C illustrates data showing sphere counts (a-c), total cell counts (d), sphere diameters (e), and percent BrdU incorporation (f), percent apoptotic cells (g) following overexpression or knockdown of MELK in adherent progenitors from E12 telencephalon (a, d-g), E15, and P0 cerebral cortecies (b and c); FIG. 32D illustrates data showing the effect of MELK for neural progenitor differentiation; as described in detail in Example 7, below.

FIG. 33 illustrates data showing MELK expression is specifically altered by the expression vector and by synthesized dsRNA. FIG. 33A illustrates data showing the expression levels of MELK in E12 progenitor cultures after transduction of various constructs; FIG. 33B illustrates data showing that while MELK expression levels were altered, neither overexpression nor siRNA targeting MELK affected the expression levels of other AMPK-family members; FIG. 33C illustrates immunocytochemistry data using anti-Flag antibody following transfection of primary progenitors with the MELK-Flag expression vector (a-c) or CRT1-Flag expression vector (d-f); FIG. 33D, treatment with MELK siRNA resulted in specific silencing of Flag expression only in those cells transfected with MELK-flag; as described in detail in Example 7, below.

FIG. 34A, FIG. 34B and FIG. 34C illustrate and summarize in graph form data demonstrating that the signaling pathway of MELK is independent of Pten-akt pathway, and is likely through a protooncogene, B-myb; as described in detail in Example 7, below.

FIG. 35A, FIG. 35B, FIG. 35C and FIG. 35D illustrate data showing that MELK upregulation is necessary for transition from GFAP-positive neural stem cells into GFAP-negative, LeX positive rapidly amplifying progenitors in vitro; as described in detail in Example 7, below.

Like reference symbols in the various drawings indicate like elements.

### DETAILED DESCRIPTION

The invention provides compositions and methods for the diagnosis, prognosis and treatment of tumors and cancers, e.g., brain cancers. In one aspect, the invention provides compositions and methods for inhibiting the growth, proliferation, differentiation and/or survival of a neural stem cell or a cancer cell, or progenitor stem cell thereof. In one aspect, the invention provides compositions and methods for identifying the genetic profile of a brain cancer cell or a self-renewing neural cancer stem cell. In one aspect, the invention provides methods employing these profiles to identify compounds that inhibit tumor growth.

### Candidate Compounds

The invention provides methods of identifying a compound that inhibits the growth, growth, proliferation, differentiation or survival differentiation or survival of a neural stem cell or a cancer or tumor cell, or a progenitor stem cell thereof (e.g., that inhibits tumor growth), comprising (a) providing a candidate compound that modulates the expression of a maternal embryonic leucine zipper kinase (MELK) gene, a T-LAK cell-originated protein kinase (TOPK) gene, a phosphoserine phosphatase (PSP) gene, a forkhead box M1 (FoxM1) gene, a B-myb gene, a Rho/Rac/Cdc42-like GTPase activating protein (RACGAP) gene, a kinesin superfamily protein member 4 (KIF4) or KIF4A gene, a cell cycle control protein CDC2 gene, a EZHa gene, a HCAP-G gene, a MCM7 gene, a CHAF1A gene, a MCM6 gene, a TMPO gene, a SPAG5 gene, a BIRC5 gene, a TYMS gene, a KPNA2 gene, a KIF2c gene, a MAD2L1 gene, a NEK2 gene, a BUB1B gene, a ECT2 gene, a UBE2C gene, a FEN1 gene, a H2AFX gene, a STK6 gene, a DNMT1 gene, a PCNA gene, a POLA gene, a TRIP13 gene, a MK167 (proliferation-related Ki-67 antigen) gene or a SLC35B1 gene, or a combination thereof; (b) contacting the cell with said compound; and (c) comparing the level of expression with a cell in the absence of the candidate compound, whereby said candidate compound is identified as a compound that inhibits cell or tumor growth as one that modulates gene expression or the activity of the encoded protein to result in cell or tumor growth inhibition.

A variety of different compounds may be identified using the method as provided herein. Compounds can encompass numerous chemical classes. In certain embodiments, they are organic molecules, preferably small organic compounds having a molecular weight of more than 50 and less than about 2,500 daltons. These compounds can comprise functional groups necessary for structural interaction with proteins, particularly hydrogen bonding, and may include at least an amine, carbonyl, hydroxyl or carboxyl group, preferably at least two of the functional chemical groups. The compounds can comprise cyclical carbon or heterocyclic structures and/or aromatic or polyaromatic structures substituted with one or more of the above functional groups.

Compounds also include biomolecules like peptides, saccharides, fatty acids, steroids, purines, pyrimidines, derivatives, structural analogs or combinations thereof.

Compounds of interest also can include peptide and protein agents, such as antibodies or binding fragments or mimetics thereof, e.g., Fv, F(ab′)₂ and Fab.

Compounds for the identification assay also can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

In one embodiment, small molecules can be used as compounds in the identification assay. Small molecule compounds include compounds that are less than about 1,000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic, acids, carbohydrates, small organic molecules (e.g., polyketides), and natural product extract libraries. In another embodiment, the compounds are small, organic non-peptidic compounds. In a further embodiment, a small molecule is not biosynthetic.

The amount of compound that is present in the contact mixture may vary, particularly depending on the nature of the compound. In one embodiment, where the agent is a small organic molecule, the amount of compound present in the reaction mixture can range from about 1 femtomolar to 10 millimolar. In another embodiment, where the agent is an antibody or binding fragment thereof, the amount of the compound can range from about 1 femtomolar to 10 millimolar. The amount of any particular compound to include in a given contact volume can be readily determined empirically using methods known to those of skill in the art.

### Cells

In alternative aspects, the invention provides, or uses, isolated or recombinant stem cells or cancer or tumor cells, e.g., neural cancer stem cells, having enriched expression of maternal embryonic leucine zipper kinase (MELK) gene. In some embodiments, the isolated cancer stem cell further comprises the enriched expression of T-LAK cell-originated protein kinase (TOPK), phosphoserine phosphatase (PSP), forkhead box M1 (FoxM1), and/or B-myb genes. In some embodiments, the isolated neural cancer stem cell also comprises a cell with enriched expression of one or more of the genes selected from the group of genes consisting of RACGAP1, KIF4A, CDC2, EZHa, HCAP-G, MCM7, CHAF1A, MCM6, TMPO, SPAG5, BIRC5, TYMS, KPNA2, KIF2c, MAD2 μl, NEK2, BUB1B, ECT2, UBE2C, FEN1, H2AFX, STK6, DNMT1, PCNA, POLA, TRIP13, MK167, and SLC35B1. These cells can be used in the assays of the invention, e.g., to determine the expression profile of a stem cell or a cancer or tumor cell to correlate the expression of a set of genes and the metastatic, growth or survival potential of a cell, or the response of a stem cell or a cancer or tumor cell to a particular treatment or set of treatments (e.g., radiation and chemotherapy, or therapy with siRNAs or oligonucleotides, or small molecules), to generate a treatment plan, diagnosis or prognosis for an individual where cell having the identified gene expression profile have been detected.

Any suitable cell, e.g., stem cell, cancer cell or tumor cell, may be employed in practicing the methods and compositions of the invention. The cell may express one or more of the genes endogenously or exogenously. Exogenous expression can be achieved using routine molecular biology techniques and can be transient, constitutive, or inducible. In one aspect, a cell employed in practicing the methods and compositions of the invention is a neural cancer stem cell, e.g., a stem cell that is CD133+. The isolated cell provided herein can be derived from (or initially derived from, in the case of a recombinant, or genetically engineered cell) a patient sample (e.g., a biopsy) or an in vitro adapted cell line. In some embodiments, the isolated cell is from a cancer or tumor sample, e.g., a pediatric tumor.

### Measuring Gene Expression

The invention provides compositions (e.g., cells) and assays to determine the expression profile of a stem cell or a cancer or tumor cell to correlate the expression of a set of genes and the metastatic, growth or survival potential of a cell, or the response of a stem cell or a cancer or tumor cell to a particular treatment or set of treatments (e.g., radiation and chemotherapy, or therapy with siRNAs or oligonucleotides, or small molecules), to generate a treatment plan, diagnosis or prognosis for an individual where cell having the identified gene expression profile have been detected. In one aspect, the methods of the invention measure the level of expression of at least one of a maternal embryonic leucine zipper kinase (MELK) gene, a T-LAK cell-originated protein kinase (TOPK) gene, a phosphoserine phosphatase (PSP) gene, a forkhead box M1 (FoxM1) gene, a B-myb gene, a Rho/Rac/Cdc42-like GTPase activating protein (RACGAP) gene, a kinesin superfamily protein member 4 (KIF4) or KIF4A gene, a cell cycle control protein CDC2 gene, a EZHa gene, a HCAP-G gene, a MCM7 gene, a CHAF1A gene, a MCM6 gene, a TMPO gene, a SPAG5 gene, a BIRC5 gene, a TYMS gene, a KPNA2 gene, a KIF2c gene, a MAD2L1 gene, a NEK2 gene, a BUB1B gene, a ECT2 gene, a UBE2C gene, a FEN1 gene, a H2AFX gene, a STK6 gene, a DNMT1 gene, a PCNA gene, a POLA gene, a TRIP13 gene, a MK167 (proliferation-related Ki-67 antigen) gene or a SLC35B1 gene, or a combination thereof, or a protein encoded by any one, several or all of these genes, wherein the level of expression of the gene is measured by determining the level of expression of the gene, a message transcribed by the gene or a protein encoded by the gene.

The level of expression of a nucleic acid or protein in a cell can be determined using any suitable method including, but not limited to RT-PCR, in situ hybridization, and intracellular flow cytometric analysis. See, e.g., Ausebel, et al., eds. CURRENT PROTOCOLS IN MOLECULAR BIOLOGY (John Wiley & Sons, 2003); Higgins, et al., eds. PROTEIN EXPRESSION: A PRACTICAL APPROACH (Oxford University Press 1999).

### Inhibiting Gene or Protein Expression

The invention provides compositions, including nucleic acids, such as siRNA or antisense oligonucleotides, polypeptides or small molecules (developed by the screening methods of the invention) for inhibiting the growth, proliferation, differentiation and/or survival of a neural stem cell or a cancer cell, or progenitor stem cell thereof, comprising at least one compound capable of (i) inhibiting transcription of a gene or inhibiting translation of a gene's transcript.

In one aspect, the gene inhibited is a maternal embryonic leucine zipper kinase (MELK) gene, a T-LAK cell-originated protein kinase (TOPK) gene, a phosphoserine phosphatase (PSP) gene, a forkhead box M1 (FoxM1) gene, a B-myb gene, a Rho/Rac/Cdc42-like GTPase activating protein (RACGAP) gene, a kinesin superfamily protein member 4 (KIF4) or KIF4A gene, a cell cycle control protein CDC2 gene, a EZHa gene, a HCAP-G gene, a minichromosome maintenance (MCM)-7 (MCM7) gene, a chromatin assembly factor-1A (CHAF-1A) gene, a minichromosome maintenance protein 6 (MCM6) gene, a thymopoietin (TMPO) gene, a sperm associated antigen 5 (SPAG5) gene, a baculoviral IAP repeat-containing 5 (BIRC5) gene, a thymidylate synthase (TYMS) gene, a karyopherin (importin) alpha 2 (KPNA2) gene, a kinesin family member 2C (KIF2c) gene, a MAD2 (mitotic arrest deficient, homolog)-like 1 (MAD2L1) gene, a NIMA (never in mitosis gene a)-related kinase 2 (NEK2) gene, a BUB1 budding uninhibited by benzimidazoles 1 homolog beta (yeast) (BUB1B) gene, an epithelial cell transforming sequence 2 oncogene (ECT2) gene, a ubiquitin-conjugating enzyme E2C (UBE2C) gene, a fatty acid elongase (FEN1) gene, a H2A histone family, member X (H2AFX) gene, a serine/threonine kinase 6 (STK6) gene, a methyltransferase Ti (DNMT1) gene, a proliferating cell nuclear antigen (PCNA) gene, a polymerase A (POLA) gene, a thyroid hormone receptor interactor 13 (TRIP13) gene, a MK167 (proliferation-related Ki-67 antigen) gene and a solute carrier family 35, member B1 (SLC35B1) gene; or (ii) inhibiting the expression or activity of protein selected from the group consisting of a maternal embryonic leucine zipper kinase (MELK), a T-LAK cell-originated protein kinase (TOPK), a phosphoserine phosphatase (PSP), a forkhead box M1 (FoxM1) protein, a B-myb protein, a Rho/Rac/Cdc42-like GTPase activating protein (RACGAP), a kinesin superfamily protein member 4 (KIF4) or KIF4A protein, a cell cycle control protein CDC2, a EZHa protein, a HCAP-G protein, a MCM7 protein, a CHAF1A protein, a MCM6 protein, a TMPO protein, a SPAG5 protein, a BIRC5 protein, a TYMS protein, a KPNA2 protein, a KIF2c protein, a MAD2L1 protein, a NEK2 protein, a BUB1B protein, a ECT2 protein, a UBE2C protein, a FEN1 protein, a H2AFX protein, a STK6 protein, a DNMT1 protein, a PCNA protein, a POLA protein, a TRIP13 protein, a MK167 (proliferation-related Ki-67 antigen) protein, a solute carrier family 35 (SLC35B1) protein, or any combination thereof. The invention provides pharmaceutical compositions comprising at least one composition of these compositions, and a pharmaceutically acceptable excipient.

The compound can modulate the expression of one of these genes by modulating expression on a transcriptional or translational level; e.g., by inhibiting the transcription of a message, decreasing the stability of a message, compartmentalizing a message such that it cannot be optimally transcribed or translated, inhibiting translation of a message, accelerating the degradation of a message, and the like. Compounds can interfere with the transcriptional activation of one or more genes. In a specific embodiment, the compound inhibits or abrogates mRNA expression. For example, the compound to inhibit or abrogate mRNA expression can be an antisense oligonucleotide, a double-stranded inhibitory RNA (RNAi) molecule, an RNase III-prepared short interfering RNA (esiRNA) or a vector-derived short hairpin RNAs (shRNA).

The nucleic acids used to practice this invention, whether RNA, iRNA (including esiRNA and shRNA), antisense nucleic acid, cDNA, genomic DNA, vectors, viruses or hybrids thereof, may be isolated from a variety of sources, genetically engineered, amplified, and/or expressed/generated recombinantly. Recombinant polypeptides (e.g., as an inhibitory compound of the invention) generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066. Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS 1N MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

Sequences of nucleic acids used to practice the invention, including the inhibitory compounds of the invention, e.g., used to inhibit or abrogate mRNA transcription or message expression, including antisense oligonucleotides, ribozymes, double-stranded inhibitory RNAs (RNAi), an RNase III-prepared short interfering RNAs (esiRNA) or vector-derived short hairpin RNAs (shRNA), are all well known in the art. For example, sequence comprising a maternal embryonic leucine zipper kinase (MELK) gene, a T-LAK cell-originated protein kinase (TOPK) gene, a phosphoserine phosphatase (PSP) gene, a forkhead box M1 (FoxM1) gene, a B-myb gene, a Rho/Rac/Cdc42-like GTPase activating protein (RACGAP) gene, a kinesin superfamily protein member 4 (KIF4) or KIF4A gene, a cell cycle control protein CDC2 gene, a EZHa gene, a HCAP-G gene, a minichromosome maintenance (MCM)-7 (MCM7) gene, a chromatin assembly factor-1A (CHAF-1A) gene, a minichromosome maintenance protein 6 (MCM6) gene, a thymopoietin (TMPO) gene, a sperm associated antigen 5 (SPAG5) gene, a baculoviral IAP repeat-containing 5 (BIRC5) gene, a thymidylate synthase (TYMS) gene, a karyopherin (importin) alpha 2 (KPNA2) gene, a kinesin family member 2C (KIF2c) gene, a MAD2 (mitotic arrest deficient, homolog)-like 1 (MAD2L1) gene, a NIMA (never in mitosis gene a)-related kinase 2 (NEK2) gene, a BUB1 budding uninhibited by benzimidazoles 1 homolog beta (yeast) (BUB1B) gene, an epithelial cell transforming sequence 2 oncogene (ECT2) gene, a ubiquitin-conjugating enzyme E2C (UBE2C) gene, a fatty acid elongase (FEN1) gene, a H2A histone family, member X (H2AFX) gene, a serine/threonine kinase 6 (STK6) gene, a methyltransferase TI (DNMT1) gene, a proliferating cell nuclear antigen (PCNA) gene, a polymerase A (POLA) gene, a thyroid hormone receptor interactor 13 (TRIP13) gene, a MK167 (proliferation-related Ki-67 antigen) gene and a solute carrier family 35, member B1 (SLC35B1) gene, are all well known in the art.

For example, the maternal embryonic leucine zipper kinase (MELK) gene transcript (message) can be found on the NCBI database as cDNA clone MGC:20350 IMAGE:4547136; and, Strausberg, et al., Proc. Natl. Acad. Sci. U.S.A. 99 (26), 16899-16903 (2002):

(SEQ ID NO:5)

MKDYDELLKYYELHETIGTGGFAKVKLACHILTGEMVAIKIMDKNTLGSD
LPRIKTEIEALKNLRHQHICQLYHVLETKIFMVLEYCPGGELFDYIISQD
RLSEEETRVVFRQIVSAVAYVHSQGYAHRDLKPENLLFDEYHKLKLIDFG
LCAKPKGNKDYHLQTCCGSLAYAAPELIQGKSYLGSEADVWSMGILLYVL
MCGFLPFDDDNVMALYKKIMRGKYDVPKWLSPSSILLLQQMLQVDPKKRI
SMKNLLNHPWIMQDYNYPVEWQSKNPFIHLDDDCVTELSVHHRNNRQTME
DLISLWQYDHLTATYLLLLAKKARGKPVRLRLSSFSCGQASATPFTDIKS
NNWSLEDVTASDKNYVAGLIDYDWCEDDLSTGAATPRTSQFTKYWTESNG
VESKSLTPALCRTPANKLKNKENVYTPKSAVKNEEYFMFPEPKTPVNKNQ
HKREILTTPNRYTTPSKARNQCLKETPIKIPVNSTGTDKLMTGVISPERR
CRSVELDLNQAHMEETPKRKGAKVFGSLERGLDKVITVLTRSKRKGSARD
GPRRLKLHYNVTTTRLVNPDQLLNEIMSILPKKHVDFVQKGYTLKCQTQS
DFGKVTMQFELEVCQLQKPDVVGIRRQRLKGDAWVYKRLVEDILSSCKV

This sequence, as all the other known sequences used to practice the invention, can be used to design and generate the inhibitory nucleic acid-based compounds used to practice the invention, e.g., the nucleic acids used to inhibit or abrogate mRNA transcription or message expression, including antisense oligonucleotides, ribozymes, double-stranded inhibitory RNAs (RNAi), an RNase III-prepared short interfering RNAs (esiRNA) or vector-derived short hairpin RNAs (shRNA). Methods for designing, making and using these inhibitory nucleic acids are well known in the art.

The nucleic acids used to practice the invention can be complementary to a sense or antisense strand (e.g., coding or non-coding strand) of a sequence used to practice the invention, e.g., a maternal embryonic leucine zipper kinase (MELK) gene, a T-LAK cell-originated protein kinase (TOPK) gene, a phosphoserine phosphatase (PSP) gene, a forkhead box M1 (FoxM1) gene, a B-myb gene, a Rho/Rac/Cdc42-like GTPase activating protein (RACGAP) gene, a kinesin superfamily protein member 4 (KIF4) or KIF4A gene, a cell cycle control protein CDC2 gene, a EZHa gene, a HCAP-G gene, a MCM7 gene, a CHAF1A gene, a MCM6 gene, a TMPO gene, a SPAG5 gene, a BIRC5 gene, a TYMS gene, a KPNA2 gene, a KIF2c gene, a MAD2L1 gene, a NEK2 gene, a BUB1B gene, a ECT2 gene, a UBE2C gene, a FEN1 gene, a H2AFX gene, a STK6 gene, a DNMT1 gene, a PCNA gene, a POLA gene, a TRIP13 gene, a MK167 (proliferation-related Ki-67 antigen) gene or a solute carrier family 35 (SLC35B1) gene (“exemplary sequences”). A sequence used to practice the invention also can be double stranded, as in some siRNAs. Nucleic acids used to practice the invention can be capable of inhibiting the transport, splicing or transcription of a gene or its transcript. The inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides which are able to either bind or cleave one of the exemplary sequences, in either case preventing or inhibiting the production or function of the protein encoded by the gene. The association can be through sequence specific hybridization.

Another useful class of inhibitors includes oligonucleotides which cause inactivation or cleavage of exemplary sequence message. The oligonucleotide can have enzyme-like activity which causes such cleavage, such as ribozymes. The oligonucleotide can be chemically modified or conjugated to an enzyme or composition capable of cleaving the complementary nucleic acid. A pool of many different such oligonucleotides can be screened for those with the desired activity. Thus, the invention provides various compositions for the inhibition of the genes encoding the exemplary genes used to practice the invention on a nucleic acid and/or protein level, e.g., antisense, iRNA and ribozymes.

Antisense Oligonucleotides

The invention provides antisense oligonucleotides capable of binding to and inhibiting the exemplary genes used to practice the invention by targeting mRNA or transcriptional regulatory sequences, e.g., promoters. Strategies for designing antisense oligonucleotides are well described in the scientific and patent literature, and the skilled artisan can design such tryptophan-processing enzyme oligonucleotides using the novel reagents of the invention. For example, gene walking/RNA mapping protocols to screen for effective antisense oligonucleotides are well known in the art, see, e.g., Ho (2000) Methods Enzymol. 314:168-183, describing an RNA mapping assay, which is based on standard molecular techniques to provide an easy and reliable method for potent antisense sequence selection. See also Smith (2000) Eur. J. Pharm. Sci. 11:191-198.

Naturally occurring nucleic acids can be used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata (1997) Toxicol Appl Pharmacol 144:189-197; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene (methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense tryptophan-processing enzyme sequences of the invention (see, e.g., Gold (1995) J. of Biol. Chem. 270:13581-13584).

Inhibitory Ribozymes

The invention provides ribozymes capable of binding exemplary sequences (e.g., exemplary genes and their messages) used to practice the invention. These ribozymes can inhibit activity by, e.g., targeting mRNA or promoters. Strategies for designing ribozymes and selecting the optimal antisense sequence for targeting are well described in the scientific and patent literature, and the skilled artisan can design such ribozymes using the novel reagents of the invention. Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it can be released from that RNA to bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The ribozyme of the invention, e.g., an enzymatic ribozyme RNA molecule, can be formed in a hammerhead motif, a hairpin motif, as a hepatitis delta virus motif, a group I intron motif and/or an RNaseP-like RNA in association with an RNA guide sequence. Examples of hammerhead motifs are described by, e.g., Rossi (1992) Aids Research and Human Retroviruses 8:183; hairpin motifs by Hampel (1989) Biochemistry 28:4929, and Hampel (1990) Nuc. Acids Res. 18:299; the hepatitis delta virus motif by Perrotta (1992) Biochemistry 31:16; the RNaseP motif by Guerrier-Takada (1983) Cell 35:849; and the group I intron by Cech U.S. Pat. No. 4,987,071. The recitation of these specific motifs is not intended to be limiting. Those skilled in the art will recognize that a ribozyme of the invention, e.g., an enzymatic RNA molecule of this invention, can have a specific substrate binding site complementary to one or more of the target gene RNA regions. A ribozyme of the invention can have a nucleotide sequence within or surrounding that substrate binding site which imparts an RNA cleaving activity to the molecule.

RNA Interference (RNAi)

In one aspect, the invention provides an RNA inhibitory molecule, a so-called “RNAi” molecule, comprising an exemplary sequence used to practice the invention. The RNAi molecule can comprise an RNase III-prepared short interfering RNA (esiRNA) or a vector-derived short hairpin RNAs (shRNA), or any double-stranded RNA (dsRNA) molecule. The RNAi can inhibit expression of an exemplary gene sequence used to practice the invention. In one aspect, the RNAi is about 15, 16, 17, 18, 19, 20, 21, 22, 23, 24, 25 or more duplex nucleotides in length. While the invention is not limited by any particular mechanism of action, the RNAi can enter a cell and cause the degradation of a single-stranded RNA (ssRNA) of similar or identical sequences, including endogenous mRNAs. When a cell is exposed to double-stranded RNA (dsRNA), mRNA from the homologous gene is selectively degraded by a process called RNA interference (RNAi). A possible basic mechanism behind RNAi is the breaking of a double-stranded RNA (dsRNA) matching a specific gene sequence into short pieces called short interfering RNA, which trigger the degradation of mRNA that matches its sequence. In one aspect, the RNAi's of the invention are used in gene-silencing therapeutics, see, e.g., Shuey (2002) Drug Discov. Today 7:1040-1046. In one aspect, the invention provides methods to selectively degrade RNA using the RNAi's of the invention. The process may be practiced in vitro, ex vivo or in vivo. In one aspect, the RNAi molecules of the invention can be used to generate a loss-of-function mutation in a cell, an organ or an animal.

Methods for making and using RNAi molecules for selectively degrade RNA are well known in the art, see, e.g., U.S. Pat. Nos. 6,506,559; 6,511,824; 6,515,109; 6,489,127. An exemplary method to make siRNA (small interfering RNA) prepared by endoribonuclease digestion (esiRNA) can be found, e.g., in Liu (2005) Dev. Growth Differ. 47:323-331; Calegari (2002) Proc. Natl. Acad. Sci. USA 99:14236-14240. An exemplary method to make vector-derived short hairpin RNAs can be found, e.g., in Fish (2004) BMC Mol. Biol. August 3; 5:9.

RNA interference is a post-transcriptional, targeted gene-silencing technique that uses double-stranded RNA (dsRNA) to degrade messenger RNA (mRNA) containing the same sequence as the dsRNA. The process occurs when an endogenous ribonuclease cleaves the longer dsRNA into shorter, 21- or 22-nucleotide-long RNAs, termed small interfering RNAs or siRNAs. As used herein, the terms “small interfering RNA” (“siRNA”) or “short interfering RNAs”) refer to an RNA (or RNA analog) comprising between about 10-50 nucleotides (or nucleotide analogs) which is capable of directing or mediating RNA interference. The smaller RNA segments then mediate the degradation of the target mRNA. Natural RNAi proceeds via fragments cleaved from free dsRNA which direct the degradative mechanism to other similar RNA sequences. RNAi occurs in cells naturally to remove foreign RNAs (e.g., viral RNAs). Alternatively, RNAi can be initiated using recombinant RNA molecules, for example, to silence the expression of target genes. See, e.g., U.S. Application No. 20040203145.

Kits for synthesis of RNAi are commercially available. Thus, for example, RNAi directed to the expression of an exemplary gene used to practice the invention, e.g., MELK, or any critical upstream or downstream effector for gene (e.g., MELK or TOPK) expression or function are contemplated. In some embodiments, the RNAi can be used to modulate gene (e.g., MELK or TOPK) and associated signaling components.

Inhibitory Proteins

Additionally, in alternative aspects, the invention provides compositions, e.g., pharmaceutical compositions, comprising at least one polypeptide or peptide compound capable of inhibiting transcription of a gene or inhibiting translation of a gene's transcript (message), or inhibiting the activity of a protein encoded by an exemplary gene used to practice the invention.

In some aspects, the polypeptide or peptide comprises an antibody. The term “antibody” includes a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

For preparation of monoclonal antibodies, any technique which provides antibodies produced by continuous cell line cultures can be used. Examples include the hybridoma technique (Kohler and Milstein, Nature, 256:495-497, 1975), the trioma technique, the human B-cell hybridoma technique (Kozbor et al., Immunology Today 4:72, 1983) and the EBV-hybridoma technique (Cole, et al., 1985, in Monoclonal Antibodies and Cancer Therapy, Alan R. Liss, Inc., pp. 77-96). Techniques described for the production of single chain antibodies (U.S. Pat. No. 4,946,778) can be adapted to produce single chain antibodies to the polypeptides encoded by exemplary genes used to practice the invention, or fragments comprising at least 5, 10, 15, 20, 25, 30, 35, 40, 50, 75, 100, or 150 consecutive amino acids thereof. Alternatively, transgenic animal, e.g., mice or goats, may be used to express human, or humanized, antibodies to these polypeptides or fragments thereof; see, e.g., U.S. Pat. No. 5,770,429.

Alternatively, an inhibitory polypeptide or peptide used to practice the invention, e.g., a polypeptide or peptide capable of binding a transcriptional activator (a promoter) of an exemplary gene used to practice the invention (e.g., MELK, TOPK, FoxM1, etc.), or polypeptide or peptide capable of binding a protein encoded by an exemplary gene used to practice the invention, can be “mimetic” or “peptidomimetic” forms. The terms “mimetic” and “peptidomimetic” refer to a synthetic chemical compound which has substantially the same structural and/or functional characteristics of natural polypeptides used to practice the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity.

### Measuring Biological Activity

In some embodiments, the compound inhibits growth, proliferation, differentiation and/or survival of a neural stem cell or a cancer or tumor cell, or progenitor stem cell thereof, by inhibiting at least one enzymatic or biological activity of a polypeptide, e.g., an enzyme or protein encoded by at least one of the following genes: a maternal embryonic leucine zipper kinase (MELK) gene, a T-LAK cell-originated protein kinase (TOPK) gene, a phosphoserine phosphatase (PSP) gene, a forkhead box M1 (FoxM1) gene, a B-myb gene, a Rho/Rac/Cdc42-like GTPase activating protein (RACGAP) gene, a kinesin superfamily protein member 4 (KIF4) or KIF4A gene, a cell cycle control protein CDC2 gene, a EZHa gene, a HCAP-G gene, a MCM7 gene, a CHAF1A gene, a MCM6 gene, a TMPO gene, a SPAG5 gene, a BIRC5 gene, a TYMS gene, a KPNA2 gene, a KIF2c gene, a MAD2L1 gene, a NEK2 gene, a BUB1B gene, a ECT2 gene, a UBE2C gene, a FEN1 gene, a H2AFX gene, a STK6 gene, a DNMT1 gene, a PCNA gene, a POLA gene, a TRIP13 gene, a MK167 (proliferation-related Ki-67 antigen) gene or a SLC35B1 gene, or a combination thereof.

For example, in one aspect the compound can inhibit phosphorylation, enzymatic activity, translocation, protein-protein interactions, and the like. Specifically, tumor inhibition can be assessed using proliferation, differentiation capacity, or self-renewal potential assays. Such assays include primary sphere formation assay, tumor stem cell proliferation, and differentiation potential assays.

Any convenient means can be used to assess the effects of the compound on one or more biological activity including, but not limited to quantified measurements of proliferation, differentiation, and stem cell renewal in vitro either when contacted with compound alone or in the presence of other relevant cell types as well as assessment in vivo. Such assessment includes assessment in the ability to inhibit in vitro proliferation, differentiation potential, or stem cell renewal potential. Models suitable for such analysis are known in the art and are exemplified by those disclosed in the Examples disclosed below.

For example, a compound is an inhibitor of a gene from a neural stem cell or a cancer or tumor cell, or progenitor stem cell thereof, when the compound reduces the incidence of cell (e.g., tumor cell) growth, proliferation, differentiation and/or survival in vitro relative to that observed in the absence of the compound. In one embodiment, the compound reduces growth or proliferation to about 5%, 10%, 20%, 25%, 30%, 40%, 50%, 55%, 60%, 65%, 70%, 75%, 80%, 85%, 90%, or 0%, of the level of growth or proliferation in the absence of the compound under the same conditions. In one aspect, the compound inhibits stem cells or a cancer or tumor cells with little to no negative effect (substantially no negative effect) on non-tumor or normal cell biological activity and/or normal growth or differentiation. In some embodiments, the compound can be assessed relative to other compounds that do not impact the biological activity of the cell being examined. In one embodiment, the compound is identified as a cell or tumor inhibitor when proliferation of the cells (e.g., neural stem or progenitor cells) in the presence of the candidate compound is about 5%, 10%, 20%, 25%, 30%, 40%, 50%, 55%, 60%, 65%, 70%, 75%, 80%, 85% or 90% less of (than) the proliferation of said neural stem or progenitor cell in the absence of the compound.

### Treating and Diagnosing Tumors and Cancers

The invention provides pharmaceutical compositions for inhibiting the growth, proliferation, differentiation and/or survival of a neural stem cell or a cancer cell, or progenitor stem cells thereof, and methods for treating cancers and tumors, including methods for inhibiting the growth, proliferation, differentiation and/or survival of a neural stem cell or a cancer cell, or progenitor stem cell thereof, in an individual in need thereof, comprising the steps of administering to the individual a therapeutically effective amount of a pharmaceutical composition of the invention.

The pharmaceutical compositions and methods of the invention are used to treat and/or assess a tumor or cancer cell, or progenitor stem cells thereof, including diagnosis or identification of the tumor, e.g., for metastatic potential, treatment (drug) sensitivity versus resistance. The tumor cells treated or assessed can be neural tumor cells or neural tumor stem cells (e.g., stem cells that can “differentiate” into cancer or tumor cells). The tumor cell can be derived from a brain tumor, e.g., a pediatric brain tumor. In some embodiments, the tumor cell is CD133 positive.

In practicing the invention, the tumor or cancer cell, or progenitor stem cells thereof, can be contacted with the compound in vivo, ex vivo and/or in vitro. In some embodiments, a neural cancer cell or a neural cancer stem cell is contacted in vivo, ex vivo and/or in vitro. In one aspect, the cell or individual treated is a mammalian cell or mammal, e.g., a human cell or human. The cell or individual treated can be contacted with any known anti-tumor, anti-differentiation or anti-proliferation agents, including but not limited to chemotherapeutic agents, radionucleotides, antibodies, and the like.

In one aspect, the invention provides compositions and methods for diagnosing the metastatic potential of a neural tumor comprising determining the presence or absence of maternal embryonic leucine zipper kinase (MELK) gene and/or protein expression. MELK expression can be determined by any suitable means including but limited to detection of MELK protein levels, MELK RNA levels, or MELK activity. A neural tumor can be diagnosed as having metastatic potential by examining a tumor sample from a patient and determining enhanced or de novo MELK expression relative to non-malignant brain tissue. Methods useful in detection of MELK levels include RT-PCR, in situ hybridization, and flow cytometric analysis. The reagents for such detection and optionally instructions for use can be provided in a kit format.

In alternative embodiments, genetically engineered viruses, e.g., lentiviruses (e.g., recombinant HIV) or adenoviruses, can be used to infect (treat) cells in vivo, ex vivo and/or in vitro to insert into the cell an inhibitory nucleic acid, e.g., an antisense oligonucleotide or an siRNA, or a nucleic acid encoding an inhibitory protein (e.g., an antibody) that is effective in the inhibition of the cell's growth or proliferation, or differentiation.

The invention provides compositions of the invention and a pharmaceutically acceptable excipient. The invention provides parenteral formulations comprising a composition of the invention. The invention provides enteral formulations comprising a composition of the invention. The invention provides methods for treating tumors, e.g., brain tumors, comprising providing a pharmaceutical composition of the invention; and administering an effective amount of the pharmaceutical composition to a subject in need thereof, thereby treating the tumor.

The pharmaceutical compositions used in the methods of the invention can be administered by any means known in the art, e.g., parenterally, topically, orally, or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, e.g., push-fit capsules made of gelatin, as well as soft, sealed capsules made of gelatin and a coating such as glycerol or sorbitol. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., an inhibitory nucleic acid or polypeptide of the invention) in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil-based pharmaceuticals are particularly useful for administration of hydrophobic active agents, including liposome comprising inhibitory nucleic acids or polypeptides used to practice the invention. Oil-based suspensions can be formulated by suspending an active agent (e.g., a chimeric composition of the invention) in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In practicing the compositions and methods of the invention, the pharmaceutical compounds can also be administered by in intranasal, intraocular and intravaginal routes including suppositories, insufflation, powders and aerosol formulations (for examples of steroid inhalants, see Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols. In practicing the compositions and methods of the invention, the pharmaceutical compounds can be delivered by transdermally, by a topical route, formulated as applicator sticks, solutions, suspensions, emulsions, gels, creams, ointments, pastes, jellies, paints, powders, and aerosols.

In practicing the compositions and methods of the invention, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In practicing the compositions and methods of the invention, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

The pharmaceutical compounds and formulations of the invention can be lyophilized. The invention provides a stable lyophilized formulation comprising a composition of the invention, which can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent app. no. 20040028670.

The compositions and formulations of the invention can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587.

The formulations of the invention can be administered for prophylactic and/or therapeutic treatments. In therapeutic applications, compositions are administered to a subject already suffering from a condition, infection or disease in an amount sufficient to cure, alleviate or partially arrest the clinical manifestations of the condition, infection or disease and its complications (a “therapeutically effective amount”). In practicing the compositions and methods of the invention, a pharmaceutical composition is administered in an amount sufficient to treat (e.g., ameliorate) or prevent a condition, diseases or symptom related to overactivity of an exemplary gene used to practice the invention, e.g., a brain tumor, a neural tumor or any other stem cell derived tumor. The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on the dosage and frequency as required and tolerated by the patient. The formulations should provide a sufficient quantity of active agent to effectively treat the treat (e.g., ameliorate) or prevent the condition, disease or symptom (e.g., stem cell growth related condition, disease or symptom). For example, an exemplary pharmaceutical formulation for oral administration of an inhibitory polypeptide or nucleic acid is in a daily amount of between about 0.1 to 0.5 to about 20, 50, 100 or 1000 or more μg per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The compositions and formulations of the invention can further comprise other drugs or pharmaceuticals, e.g., compositions for treating tumors and cancers, e.g., of neural origin, and related symptoms or conditions. The methods of the invention can further comprise co-administration with other drugs or pharmaceuticals, e.g., compositions for treating tumors and cancers, e.g., of neural origin, and related symptoms or conditions. For example, the methods and/or compositions and formulations of the invention can be co-formulated with and/or co-administered with anticancer agents, antibiotics (e.g., antibacterial or bacteriostatic peptides or proteins), fluids, cytokines, immunoregulatory agents, anti-inflammatory agents, complement activating agents, such as peptides or proteins comprising collagen-like domains or fibrinogen-like domains (e.g., a ficolin), carbohydrate-binding domains, and the like and combinations thereof.

The invention provides means of in vivo delivery of nucleic acids used to practice the invention; wherein in one aspect the nucleic acid is operatively linked to a promoter constitutively or inducibly active in a neuron, a brain cell, or a stem cell, or a tumor cell. In one aspect, the invention uses vector constructs that are targeted for delivery and/or expression in a neuron, a brain cell, or a stem cell, or a tumor cell. In another aspect, the invention uses vector constructs that are not otherwise targeted for delivery and/or expression that is restricted to a neuron, a brain cell, or a stem cell, or a tumor cell, but rather are “anatomically” directed by injection of the vector into a blood vessel directly supplying the desired tissue target, e.g., a tumor, the brain, and the like, e.g., by injection into the carotid artery. Such injection can be achieved by catheter introduced substantially (typically at least about 1 cm) within the ostium of the anatomically advantageous artery or vein or other conduits delivering blood to the tumor or brain. By injecting a vector into the lumen of an artery, in one aspect an amount of about 10⁷ to 10¹³ nucleic acid expression particles (e.g., viral particles, such as engineered viruses, e.g., lentiviruses, such as recombinant HIV) as determined by optical densitometry are delivered to maximize therapeutic efficacy of gene transfer, and minimizing undesirable effects at non-desired sites.

Vector constructs, e.g., engineered lentiviruses or adenoviruses, that are specifically targeted to a desired cell or tissue, e.g., a cancer or tumor, a neuron or the brain can be used in place of or, depending on the application, preferably, or in addition to, such directed injection techniques as a means of further restricting expression to the desired tissue. For vectors that can elicit an immune response, it is preferable to inject the vector directly into a blood vessel, as described above. Such vector constructs and viral delivery vehicles are well known in the art, see, e.g., U.S. Pat. No. 6,579,855, describing an adenovirus having a functional thymidine kinase gene is useful in the treatment of brain tumors. Methods and compositions useful for enhancing the diffusion of gene therapy vectors through a mammalian tissue of interest, e.g., a brain, can also be used, see, e.g., U.S. Pat. No. 6,794,376. U.S. Pat. No. 6,683,058, describes use of an adeno-associated viral (AAV) vector with an operable transgene that is effective in expressing a recombinant protein (encoded by the vector) after delivery to the brain and to the CNS for up to 12 months. As such, a long term (chronically available) source of inhibitory nucleic acid or polypeptide is provided to the targeted tissue, e.g., the brain.

The invention can also be practiced using techniques for direct in vivo electrotransfection, e.g., as described in U.S. Pat. No. 6,519,492, describing method for direct in vivo electrotransfection of a plurality of cells of a target tissue (e.g., a cancer) where the target is perfused with a transfection solution. An exterior electrode is positioned so as to surround at least a portion of the target tissue. One or more interior electrodes are placed within the target tissue. The perfusion and the application of the interior and exterior electrodes may be performed in any particular order. After the perfusion and the positioning of the electrodes, both interior and exterior, an electric waveform is applied through the exterior electrode and the interior electrode to transfect the cells in the target tissue.

Pharmaceutical compositions of the invention can be administered intracranially using intracranial implants, which are well known in the art, as intracranial implants have been used for various conditions. For example, stereotactically implanted, temporary, iodine-125 interstitial catheters can be used to treat malignant gliomas; see, e.g., Scharfen, et al., High Activity Iodine-125 Interstitial Implant For Gliomas, Int. J. Radiation Oncology Biol Phys 24(4); 583-591:1992. Permanent, intracranial, low dose ¹²⁵I seeded catheter implants have been used to treat brain tumors, see, e.g., Gaspar, et al., Permanent ¹²⁵I Implants for Recurrent Malignant Gliomas, Int J Radiation Oncology Biol Phys 43(5); 977-982:1999. See also chapter 66, pages 577-580, Bellezza D., et al., Stereotactic Interstitial Brachytherapy, in Gildenberg P. L. et al., Textbook of stereotactic and Functional Neurosurgery, McGraw-Hill (1998). Pharmaceutical compositions of the invention can be stereotactically implanted using analogous techniques. See, e.g., U.S. Pat. No. 6,921,538.

Pharmaceutical compositions of the invention can be administered by local, intracranial delivery to provide a high, local therapeutic level of the toxin and can significantly prevent the occurrence of any systemic toxicity. A controlled release polymer capable of long term, local delivery of pharmaceutical compositions of the invention to an intracranial site can circumvent the restrictions imposed by systemic toxicity and the blood brain barrier, and permit effective dosing of an intracranial target tissue. An exemplary implant is described in U.S. Pat. No. 6,306,423, describing the direct introduction of a chemotherapeutic agent to a brain target tissue via a controlled release polymer. The implant polymers used can be hydrophobic so as to protect the polymer incorporated polypeptide or nucleic acid from water induced decomposition until the toxin is released into the target tissue environment.

Surgically implanted biodegradable implants can be utilized to locally administer the anti-cancer pharmaceutical compositions of the invention. For example, polyanhydride wafers containing 3-bis(chloro-ethyl)-1-nitrosourea (BCNU) (Carmustine) can be used as intracranial implants; e.g., as described by Brem, H. et al., The Safety of Interstitial Chemotherapy with BCNU-Loaded Polymer Followed by Radiation Therapy in the Treatment of Newly Diagnosed Malignant Gliomas Phase I Trial, J Neuro-Oncology 26:111-123:1995.

The target sites for administration of the neurotoxin to the patient may be targeted by using a stereotactic placement apparatus. For example, an implant or a needle containing pharmaceutical compositions of the invention can be stereotactically placed at a desired target site using the Riechert-Mundinger unit and the ZD (Zamorano-Dujovny) multipurpose localizing unit. A contrast-enhanced computerized tomography (CT) scan, injecting 120 ml of omnipaque, 350 mg iodine/ml, with 2 nm slice thickness can allow three dimensional multiplanar treatment planning (STP, Fischer, Freiburg, Germany). This equipment permits planning on the basis of magnetic resonance imaging studies, merging the CT and MRI target information for clear target confirmation.

Other stereotactic systems may also be used, including for example, the Leksell stereotactic system (Downs Surgical, Inc., Decatur, Ga.) modified for use with a GE CT scanner (General Electric Company, Milwaukee, Wis.) as well as the Brown-Roberts-Wells (BRW) stereotactic system (Radionics, Burlington, Mass.). The annular base ring of the BRW stereotactic frame can be attached to the patient's skull. Serial CT sections can be obtained at 3 mm intervals though the (target tissue) region with a graphite rod localizer frame clamped to the base plate. A computerized treatment planning program can be run on a VAX 11/780™ computer (Digital Equipment Corporation, Maynard, Mass.) using CT coordinates of the graphite rod images to map between CT space and BRW space.

Pharmaceutical compositions of the invention can be administered by use of permeable device or containers, e.g., coated, apertured containers, permeable to water but only semi-permeable to the pharmaceutical composition; e.g., as described in U.S. Pat. No. 6,669,954. An excipient formulation can also be in the container, e.g., comprising a biocompatible polymer, e.g., a hard gelatin capsule. The excipient formulation can include release control components, filling agents and lubricating agents. The container can be coated with a covering permeable to water but only semi-permeable to the pharmaceutical agent in the container. The covering may optionally include cellulose acetate.

In delivering pharmaceutical compositions of the invention, compositions for increasing cerebral bioavailability also can be used, e.g., by administering the pharmaceutical compositions of the invention while increasing brain NO levels, e.g., as described in U.S. Pat. No. 6,818,669. This increase in NO levels can be accomplished by stimulating increased production of NO by eNOS, especially by administering L-arginine, by administering agents that increase NO levels independent of ecNOS, or by any combination of these methods. As NO is increased, cerebral blood flow is consequently increased, and drugs in the blood stream are carried along with the increased flow into brain tissue. By increased flow, the site of action will be exposed to more drug molecules. By stimulating increased NO production, administration of drugs that are not easily introduced to the brain may be facilitated and/or the serum concentration necessary to achieve desired physiologic effects may be reduced.

Unless defined otherwise, all technical and scientific terms used herein have the same meaning as is commonly understood by one of ordinary skill in the art to which this invention belongs. All patents, published patent applications and other publications and sequences from GenBank and other databases referred to herein are incorporated by reference in their entirety. If a definition set forth in this section is contrary to or otherwise inconsistent with a definition set forth in patents, published patent applications and other publications and sequences from GenBank and other data bases that are herein incorporated by reference, the definition set forth in this section prevails over the definition that is incorporated herein by reference.

Other features and advantages of the invention will be apparent from the detailed description, and from the claims. As used herein, “a” or “an” means “at least one” or “one or more.”

The following examples are offered to illustrate but not to limit the invention.

### EXAMPLES

### Example 1

### MELK/MPK38 Regulates Multipotent Neuronal Progenitor Cell Self-Renewal

The invention provides a screening strategy to identify multipotent progenitor cells (MPCs), including neural progenitor cells (NPCs), that may be involved in uncontrolled cell growth, e.g., the MPCs or NPCs may be or develop into cancer or tumor cells or their progenitor cells. For example, the invention provides methods for inhibiting the growth, proliferation, differentiation and/or survival of a neural stem cell or a cancer cell, or progenitor stem cell thereof, in an individual in need thereof, comprising the steps of administering to the individual a therapeutically effective amount of a pharmaceutical composition of the invention, which include nucleic acids that inhibit the expression of a gene differentially expressed in a neural progenitor cell (NPC), including, e.g., a sequence comprising a maternal embryonic leucine zipper kinase (MELK) sequence, a T-LAK cell-originated protein kinase (TOPK) sequence, a phosphoserine phosphatase (PSP) sequence, a forkhead box M1 (FoxM1) sequence, a B-myb sequence, a Rho/Rac/Cdc42-like GTPase activating protein (RAC GAP) sequence, a kinesin superfamily protein member 4 (KIF4) or KIF4A sequence, a cell cycle control protein CDC2 sequence, a EZHa sequence, a HCAP-G sequence, a MCM7 sequence, a CHAF1A sequence, a MCM6 sequence, a TMPO sequence, a SPAG5 sequence, a BIRC5 sequence, a TYMS sequence, a KPNA2 sequence, a KIF2c sequence, a MAD2 μl sequence, a NEK2 sequence, a BUB1B sequence, a ECT2 sequence, a UBE2C sequence, a FEN1 sequence, a H2AFX sequence, a STK6 sequence, a DNMT1 sequence, a PCNA sequence, a POLA sequence, a TRIP13 sequence, a MK167 (proliferation-related Ki-67 antigen) sequence or a solute carrier family 35 (SLC35B1) sequence, or a combination thereof.

Genome-wide screening strategy has suggested some genes that may regulate neural progenitor cell (NPC) function. See, e.g., Easterday, M. C., et al., Dev. Biol. Res. (2003) 264:309-322; Geschwind, D. H., et al., Neuron (2001) 29:325-339; Karsten, S., L., et al., Dev. Biol. (2003) 261:165-182; Terskikh, A. V., et al., Proc. Natl. Acad. Sci. USA (2001) 98:7934-7939. Genes expressed by neural stem/progenitor cell populations and not differentiated cells would be those involved in self-renewal, a fundamental feature of neural progenitor cell (NPC).

To identify such genes, a custom, subtracted cDNA microarray was used to identify genes expressed in multiple NSC-containing neurospheres. Screening in situ hybridization analysis was then used to narrow this pool of genes by determining which ones were highly expressed in developing germinal zones in vivo. See, e.g., Easterday, supra (2003). Numerous genes that are enriched in neural progenitors were identified. Many of these genes were expressed within CNS germinal zones in vivo, and thus were candidates for playing roles in MPC function. See, e.g., Geschwind, supra (2001); Easterday, supra (2003).

MELK, also known as MPK38 was present in multiple NSC-containing populations and in hematopoietic stem cells. See, e.g., Gil, M., et al., Gene (1997) 195:295-301; Heyer, B. S., et al., Dev. Dyn. (1999) 215:344-351; Heyer, B. S., et al., Mol. Reprod. Dev. (1997) 47:148-156. MELK is a member of the snf1/AMPK family of kinases. Although several members of the family are known to play roles in cell survival under metabolically challenging conditions, the function of MELK has not previously been determined. See, e.g., Inoki, K., et al., Cell (2003) 115:577-590; Kato, K., et al., Oncogene (2002) 21:6082-6090; Suzuki, A., et al., Oncogene (2003a) 22:6177-6182; Suzuki, A., et al., J. Biol. Chem. (2003b) 278:48-53.

These results demonstrated that MELK regulated the proliferation of progenitor cells derived from ependymomas. Treatment with MELK siRNA inhibited the formation of spheres derived from ependymoma progenitors.

Materials and Methods

Neural progenitor cultures. Neurosphere cultures were prepared as described previously. Cortical telencephalon was removed from E12 CD-1 mice, and cerebral cortex was isolated from E15 and P0 (Charles River). Cells were dissociated with a fire-polished glass pipette, and resuspended at 50,000 cells per ml in DMEM/F12 medium (Invitrogen) supplemented with B27 (Gibco BRL), 20 ng/ml basic fibroblast growth factor (bFGF) (Peprotech), and penicillin/streptomycin (Gemini Bioproducts) and heparin (Sigma). Growth factors were added every 3 days. For differentiation, culture medium was replaced into Neurobasal (Invitrogen) supplemented with B27 without FGF onto poly-L-lysine (PLL)-coated dishes, and maintained up to 5 days. For secondary sphere formation assay, the primary spheres were dissociated and plated into 96-well microwell plates in 0.2 ml volume of growth media including conditioned media at 40,000 cells per milliliter, and the resultant sphere numbers were counted at 7 days.

To assay the influence of gene knockdown or overexpression, the neurosphere culture system was modified. Neurospheres were propagated for 1 week and then dissociated with trypsin (0.05%) followed by trituration with a fire-polished pipette. The cells were then placed in DMEM/F12 with 2% fetal bovine serum (Gibco BRL #26140-079, Carlsbad) and plated onto polyornithine/fibronectin coated glass coverslips (Sun Y., et al., Cell (2001) 104:365-376). After 6 hours, the serum-containing medium was removed and the cells were placed back in the neurosphere growth medium without heparin and supplemented with bFGF (20 ng/ml). Transfection was then performed as described below. To assay the sphere-forming potential of the transfected cells, they were lifted off the plate with trypsin (0.05%) and then placed into Neurobasal media supplemented with B27, bFGF and heparin (Wachs, F. P., et al., Lab. Invest. (2003) 83:949-962). To assay the function of cells expressing EGFP driven by the MELK promoter, 1 week neurospheres were plated onto coverslips as above and transfected. Some cultures were then placed into neurosphere conditions to assay sphere-forming potential, while others were propagated and differentiated on the coated coverslips after transfection. Proliferation activity was measured by BrdU incorporation for 24 hours at DIV3, which is shown as O.D. 492 nm, using Cell Proliferation ELISA, BrdU (colorimetric) kit (Roche), according to manufacturer's protocol.

GFAP-positive astrocyte-enriched cultures. Primary astrocyte cultures were prepared from P1 mouse cortices as described previously (Imura, T., et al., J. Neurosci. (2003) 23:2824-2832). Briefly, as cells became confluent (12-14 DIV), they were shaken at 200 rpm overnight to remove nonadherent cells and obtain pure astrocytes, and passaged on PLL-coated coverslips for RNA collection or FGF stimulation. To determine the expression and function of MELK during the production of neural stem cells from astrocyte-like progenitors, the media were changed to neurosphere growth medium with heparin.

Quantitative and Semiquantitative RT-PCR. Total RNA was isolated from each sample using TRIzol (GIBCO BRL), and 1 ug of RNA was converted to cDNA by reverse transcriptase following the manufacturer's protocol (Impron). For semi-quantitative RT-PCR, the amount of cDNA was examined by RT-PCR using primers for glyceraldehyde-3-phosphate-dehydrogenase gene (GAPDH) as an internal control from 20 to 45 cycles. After correction for the amount of GAPDH amplicons for each set, the resultant cDNA was subjected to PCR analysis using gene-specific primers listed in FIG. 15A. The protocol for the thermal cycler was: denaturation at 94° C. for 3 min, followed by corresponding cycles of 94° C. (30 sec), 60° C. (1 min), and 72° C. (1 min), with the reaction terminated by a final 10 min incubation at 72° C. Control experiments were done either without reverse transcriptase and/or without template cDNA to ensure that the results were not due to amplifications of genomic or contaminating DNA. Each reaction were visualized after 2% agarose gel electrophoresis for 30 min, and the expression levels were compared between the cDNA samples on a same gel.

For quantitative RT-PCR. DNase treated RNA samples (1 μg) were directly reverse transcribed with ImPromt-II RT™ (Promega). Real-time PCR was performed utilizing a LightCycler rapid thermal cycler system (Roche Diagnostics) according to the manufacturer's instructions. A master mix of the following reaction components was prepared to the indicated end-concentrations: 8.6 μl of water, 4 μl of Betaine (1 M), 2.4 μl of MgCl₂ (4 mM), 1 μl of primer mix (0.5 μM) and 2 μl LightCycler (Fast Start DNA Master SYBR Green I: Roche Diagnostics). LightCycler MASTERMIX™ (18 μl) was filled in the LightCycler glass capillaries and 2 μl cDNA was added as PCR template. A typical experimental run protocol consisted of an initial denaturation program (95° C. for 10 min), amplification and quantification program repeated 45 times (95° C. for 15 s, 62° C. for 5 s, 72° C. for 15s followed by a single fluorescence measurement). Relative quantification is determined using the LightCycler Relative Quantification Software (Roche Diagnostics), which takes the crossing points (CP) for each target transcript and divides them by the reference GAPDH CP.

Immunocytochemistry. Immunocytochemistry of neurospheres, adherent progenitors, and neonatal astrocytes were performed as described previously (Geschwind, supra (2001)). Cells were fixed with 3% paraformaldehyde (PFA) for 30 minutes and immunostained with the following primary antibodies: nestin (Rat401; 1:200; Developmental Studies Hybridoma Bank), LeX (CD15; 1:200; Invitrogen), TuJ1 (1:500, Berkeley Antibodies), GFAP (1:1000, DAKO), and O4 (1:50, Chemicon). Primary antibodies were visualized with Alexa 568 (red), 488 (green) and 350 (blue) conjugated secondary antibodies (Molecular Probes). Hoechst 333342 (blue) and PI (red) were used as a fluorescent nuclear counterstain.

Sphere Diameter Analysis. Secondary neurospheres from E12.5 telencephalon were plated into coverslips and fixed with 4% PFA. Diameters of 30-120 randomly chosen spheres from each condition were measured using the Microcomputer Imaging Device Program (MCID). A minimum cutoff of 40 um was used in defining a neurosphere.

Construction of Vectors.

pCMY-MELK The full-length coding region of mouse MELK was amplified by PCR using mouse embryonic neurospheres as a template, and subcloned into TEasy vector (Promega). After sequence verification, MELK fragment was subcloned into pCMV-tag vector (STRATAGENE) at NotI site.

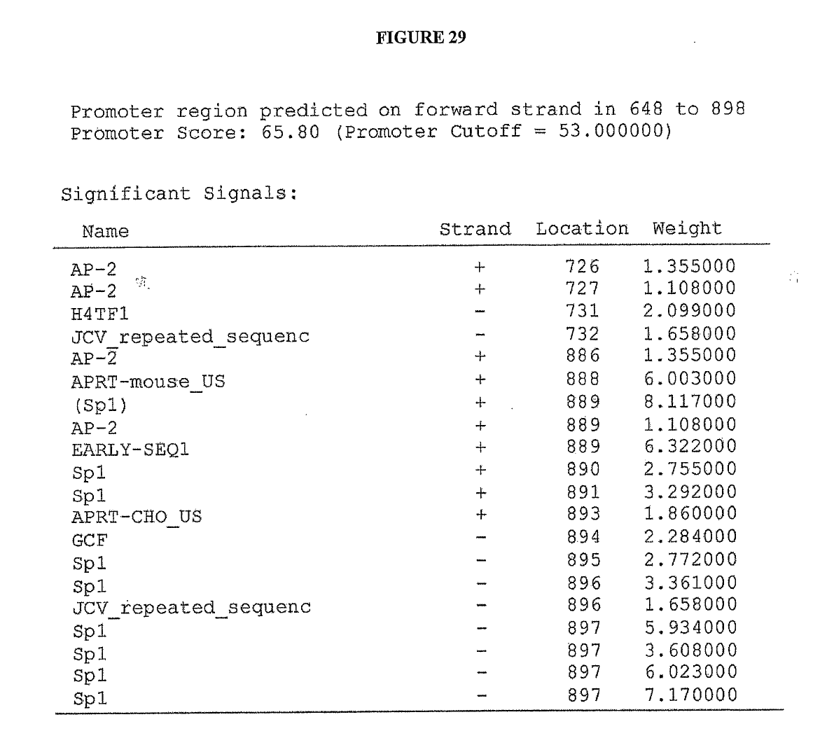

PMELK-EGFP. The putative MELK promoter region was defined using PROMOTERSCAN™ (http://bimas.dcrt.nih.gov/molbio/proscan/). This program indicated that the 2.7 kb upstream of the starting ATG codon had multiple transcription factor binding sequences as is shown in FIG. 29. A bacterial artificial chromosome (BAC) clone was obtained from BAC/PAC resources (Children's Hospital Oakland Research Institute in Oakland). Using this BAC clone as a template, 3.5 kb and 0.7 kb upstream of the starting ATG codon of mouse MELK was amplified and subcloned into Teasy vector. After the sequence confirmation, a genomic region of MELK promoter was fused to EGFP polyA (Clontech) yielding PMELK-EGFP.

siRNA Synthesis. siRNA was synthesized using the Silencer siRNA Construction Kit following manufacturer's instruction (Ambion). Four different targeting sequences were designed from coding region of mouse MELK. Each of the four demonstrated different levels of mRNA knockdown, and one was chosen for further analysis. Its targeting sequences are as follows: MELK specific siRNA, AACCCAAGGGCAACAAGGAdTdT (SEQ ID NO:1).

Flow Cytometry and Sorting. Flow cytometry and sorting of EGFP+ cells from E12- and E15-derived neural progenitors were performed on a FACS Vantage (Becton-Dickinson) using a purification-mode algorithm. Gating parameters were set by side and forward scatter to eliminate dead and aggregated cells, and EGPF vector without promoter transfected cells were used for a negative control to set the background fluorescence; false positive cells were less than 0.5%. For isolation of LeX+ cells (Capela, A., et al., Neuron (2002) 35:865-875), E12 progenitors were labeled with LeX antibody (Invitrogen) for 30 minutes and Alexa 530 was used for flow cytometry and sorting. Background signals were investigated by the same set of progenitors without primary antibody.

Transient Transfection. Cells were transfected using LIPOFECTAMINE 2000™ (Invitrogen) following manufacturer's protocol. For transfection of plasmid vectors, the cells were incubated with reagents for 6 hours with the primary progenitor cells, and for 24 hours with N2a cells. For transfection of the double stranded siRNA complex, serial doses of siRNA from 5 to 200 nM were tested to obtain specific knockdown of the gene of interest, and 100 nM was chosen as the concentration for functional study. Incubation with siRNA complex was 6 hours with primary cells and 24 hours with cell lines.

Results

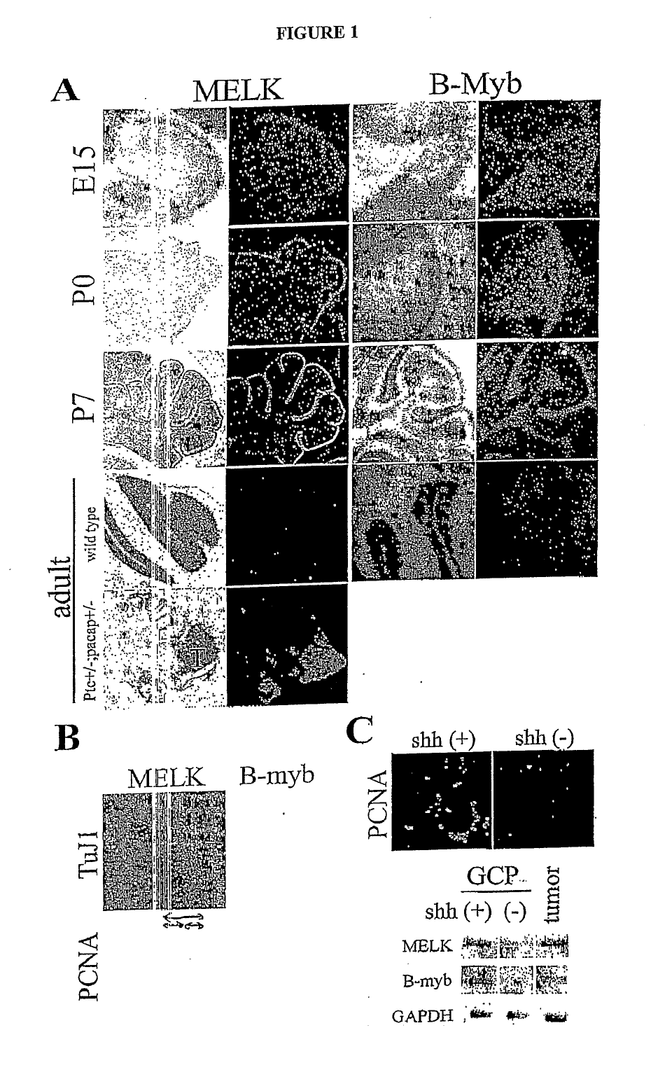

Both MELK and B-myb were highly expressed in proliferating granule cell precursors in the neonatal brains. MELK was previously identified as highly enriched in the germinal zones in the developing brains and as a regulator of self-renewal for multipotent progenitor cell (MPC) in vitro. This study focused on MELK in the developing cerebellum and medulloblastoma formed from this region. By using double labeling with in situ hybridization (ISH) and immunohistochemistry, the cell types which express MELK and its putative downstream proto-oncogene, B-myb, were investigated. Throughout the ontogeny, a striking similarity was observed in the expression of these genes in the cerebellum, as illustrated in FIG. 1. These genes were faintly expressed in the rhombic tip at E13, and after birth, they were only detected in the granule cell layer (GCL). By double-labeling with TuJ1, MELK was found exclusively in the precursor population in the outer granule cell layer, but not in the premigratory postmitotic neurons, see FIG. 1. These data was supported by RT-PCR of MELK using cell culture of granule cell precursors (GCP). With its mitogen, sonic hedgehog, GCL's keep proliferating, while a withdrawal of this mitogen ceases it. MELK and B-myb expression were both identified only in proliferating GCP's, see FIG. 1. These data suggested that MELK and B-myb were exclusively expressed in the proliferating GCP's.

FIG. 1A illustrates: In situ hybridization (ISH) of MELK and B-myb during development. FIG. 1B illustrates: Dual labeling of ISH with immunohistochemistry in the granule cell layer of the cerebellum at P7. The Immature premigratory neurons are labeled with TuJ1, and proliferating precursors are labeled with PCNA. Signals for MELK and B-myb are shown as black dots. FIG. 1C illustrates: Granule cell precursors at P7 are cultured with or without sonic hedgehog. Immunocytochemistry with PCNA is shown in 1A, and RT-PCR for MELK and B-myb is shown in 1B. GAPDH is used for an internal control. A tumor sample from Ptc/pacap mice is used as a positive control.

MELK is highly expressed in spontaneous cerebellar tumors in Ptc+/−; pacap+/−mice, and regulates its tumor growth in vitro. Medulloblastoma is the most common pediatric brain tumor in the cerebellum, which is considered to be formed from GCP's. Sonic hedgehog (shh) signaling is one of the key cascades which regulates proliferation of both GCP and medulloblastoma, and heterozygous mice of Patched, antagonistic membrane protein against shh, form spontaneous tumors in the cerebellum with a high frequency when they are crossed with heterozygous mice of pacap. These mouse tumors resemble to human medulloblastoma in regard to the histology and the affected region.

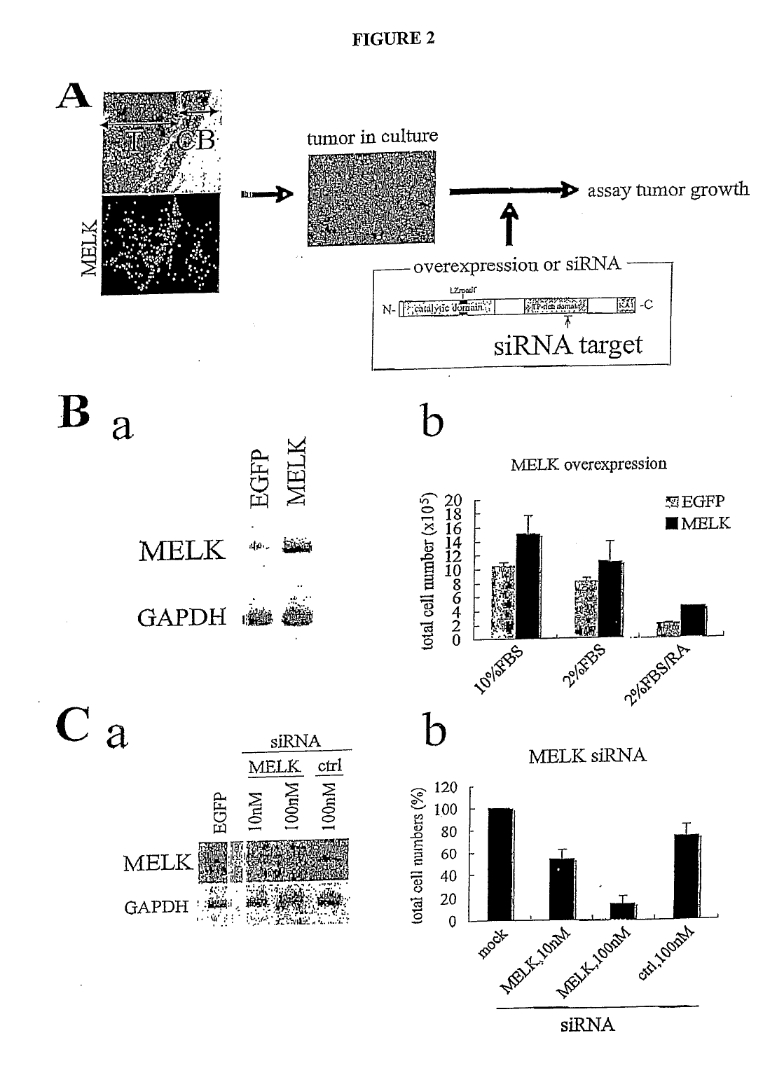

To investigate a role of MELK in medulloblastoma, MELK expression by in situ hybridization was examined using a cerebellum bearing a spontaneous tumor. As is shown in FIG. 2A, panel d, MELK was strongly expressed in the tumor cavity but not in the normal cerebellum. In a magnified view (f), a clear border of MELK expression was seen at the edge of the tumor. To investigate MELK function in these tumors, double-strand RNA (siRNA) targeting MELK, see FIG. 2B, and B-myb were designed and synthesized. With siRNA treatment for these tumor cultures, gene doses and protein expression were examined whether or not the treated siRNA affected expression level of MELK and B-myb in these tumor cells. RT-PCR and immunocytochemistry studies showed that both MELK and B-myb were down-regulated, see FIG. 2C. After the confirmation of downregulation of these two genes expression, the treated tumor cultures were maintained for 5 days, and proliferation analysis performed. Total cell numbers in each condition showed that downregulation of both MELK and B-myb resulted in inhibition of tumor growth in vitro.

FIG. 2 illustrates data demonstrating that MELK is highly expressed in spontaneous cerebellar tumors in Ptc/pacap mice, and regulates its tumor growth in vitro. FIG. 2A illustrates: A photograph of cerebellum of Ptc/pacap mouse (a). MicroPET scan of a Ptc/pacap mouse bearing tumors (b). ISH of MELK using a mouse with a tumor (d), and cresyl violet staining of adjacent slice (c). FIG. 2B illustrates: Overexpression of MELK into Ptc/pacap tumor cells in culture. MELK expression was examined in tumors after transfection (a). Five days after transfection, total cells were counted in both EGFP expressing tumor cells and MELK expressing tumor cells. FIG. 2C illustrates: A schema showing MELK structure and a target for mouse siRNA. RT-PCR with the tumor cells using MELK primers after treatment of siRNA for MELK. siRNA treated tumor cells were cultured for five days and the resultant total cell number was counted for each condition. T-test. The data is based on three independent experiments. Abbreviation; FBA; fetal bovine serum, RA; retinoic acid.

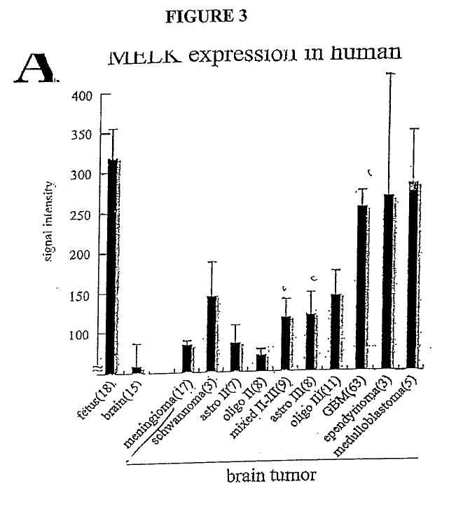

MELK is highly expressed in human medulloblastoma, and regulates its proliferation in vitro. As MELK was highly expressed in mouse medulloblastoma and regulated its proliferation, we examined MELK expression using multiple human samples. Among 229 human samples including 116 primary brain tumors, MELK expression was compared by signal intensity on cDNA microarray slides, and as a result, the cell/tumor type with the highest MELK expression was normal fetus followed by medulloblastoma, as illustrated in FIG. 3 (FIG. 3A). MELK expression level was compared among 96 normal human samples and 128 brain tumor samples based on microarray results. The number for each condition represents the number of samples.

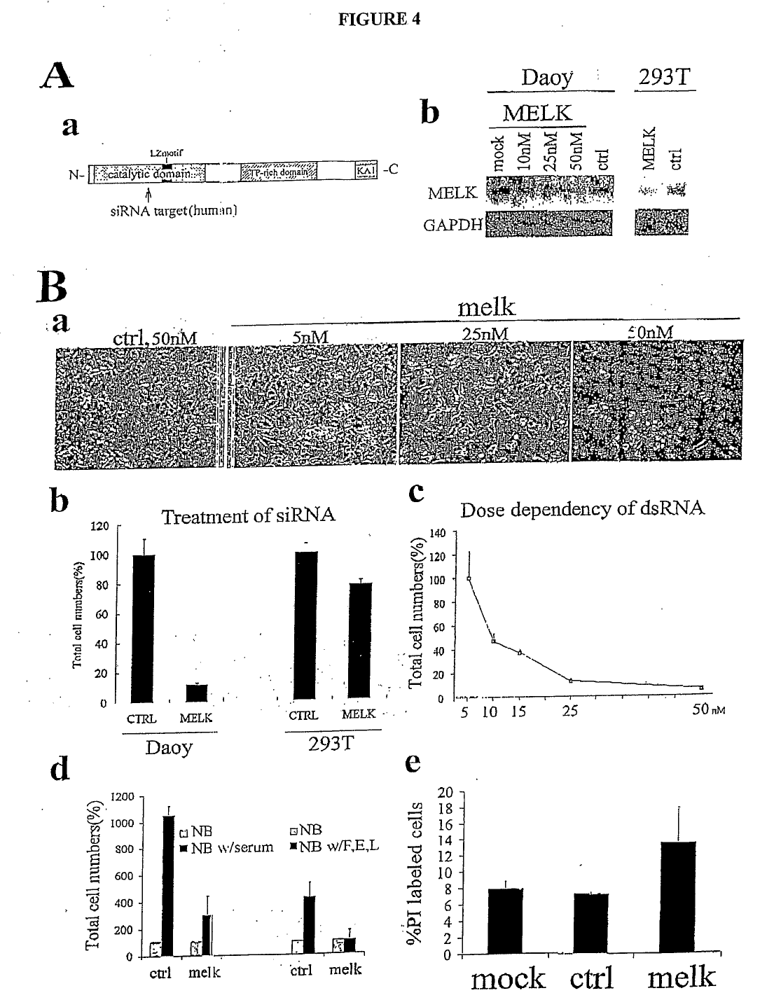

To examine MELK function in human medulloblastoma, another siRNA targeting different region of MELK was designed and synthesized, as illustrated in FIG. 4Aa, and siRNA was treated for human medulloblastoma cell line, Daoy. RT-PCR confirmed knockdown of MELK dose dependently, as illustrated in FIG. 4Ab. To investigate the effect of MELK for medulloblastoma in culture, the siRNA was treated for Daoy cells and cell growth after the treatment was observed for each condition. As a control, human fibroblast cell line, 293T cells, was also treated with siRNA for MELK. The results in FIG. 4Ba, FIG. 4Bb, FIG. 4Bc and FIG. 4Bd, show that MELK siRNA inhibited Daoy cell growth but not 293T cell growth in a dose-dependent manner, which indicated that MELK is not the general regulator of cell growth, but rather is required for the growth of medulloblastoma proliferation. We then examined whether the effect of MELK siRNA is for cell survival or cell proliferation.

FIG. 4 illustrates data demonstrating that RNAi treatment targeting MELK inhibits human medulloblastoma growth in vitro. FIG. 4A illustrates: A schema showing the target of siRNA (a). Note that different region was chosen from the target of mouse MELK. FIG. 4B illustrates: RT-PCR showing the alteration of MELK expression in Daoy cells and 293T cells by siRNA for MELK (b). FIG. 4B illustrates: Pictures of siRNA treated Daoy cells (a) and the resultant total cell numbers (b and c) are shown after treatment for five days. The graph in c shows the dose dependent effect of siRNA for tumor growth in culture. Human fibroblastoma cell line, 293T, was used as a control. Propium iodide-labeled tumor populations were measured for cell death assay after treatment of siRNA for two days (e).

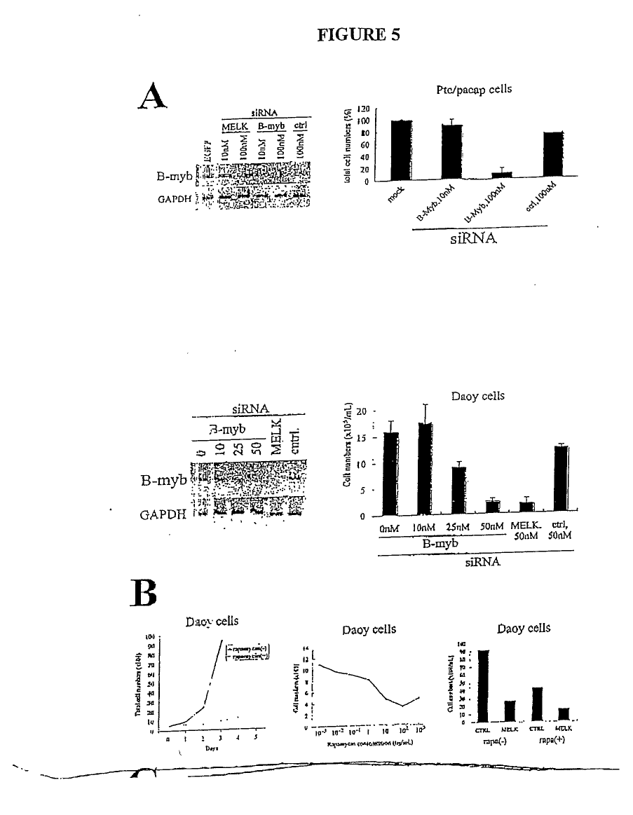

Signaling of MELK is not dependent on sonic hedgehog or akt-mTOR. One of the major signaling cascade regulating medulloblastoma proliferation is shh-Gli1 signaling, and recent investigations suggest that cyclopamine, an inhibitor for this signaling pathway, can block medulloblastoma in vivo and in vitro. Therefore, the effect MELK siRNA together with cyclopamine was tested in order to investigate if MELK effect for medulloblastoma proliferation is through shh-Gli1 signaling cascade, see FIG. 5A. By treating Daoy cells with cyclopamine, inhibition of cell growth was observed, and a combination of cyclopamine and MELK siRNA, but not control siRNA, showed additive effect for Daoy cell proliferation. These data indicated that MELK regulates medulloblastoma proliferation independently from shh-Gli1 signaling pathway. Next, akt-mTOR pathway was tested by using mTOR inhibitor, rapamycin, see FIG. 5B. Phospho-S6 is the downstream protein of mTOR, and immunostaining with antibody against phospho-S6 confirmed the inhibition of downstream signaling by rapaamycin.

FIGS. 5A and 5B illustrate data showing signaling of MELK is not dependent on sonic hedgehog or akt-mTOR. B. Treatment of Daoy cells were cultured with or without mTOR inhibitor, rapamycin for up to five days, and the effect was measured by counting the total cell number (a). The graph in b shows the effect of rapamycin with different doses. Combination of treatment by MELK siRNA and rapamycin against Daoy cells in culture (c). After siRNA was treated for Daoy culture, rapamycin was added, and the tumor cells were incubated for four days.

### Example 2

### Identification of Candidates Genes in Neural Stem Cell Self-Renewal

The microarray studies of neural stem cell genes revealed candidates that regulate neural stem cell self-renewal. Functional testing in neural stem cell cultures were initiated on the following genes. See e.g., Geschwind, supra (2001); Terskikh, supra (2001); Easterday, supra (2003); Karsten, supra (2003). Using RNA interference or pharmacological inhibitors, the following genes were demonstrated to regulate neural stem cell proliferation in vitro:

| | Reference | Reference | | |
|---|---|---|---|---|
| Gene | (identification) | (function) | Methods | Effect |
| MELK | Easterday, 2003 | (Nakano) | siRNA | Pos. regulator of cell cycle |
| TOPK | Easterday, 2003 | (Dougherty) | Pharmacological | Pos. regulator of cell cycle |
| | | | inhibition | |
| PSP | Geschwind, 2001 | NA | siRNA | Pos. regulator of cell cycle |
| | Terskikh, 2001 | | | |
| | Easterday, 2003 | | | |
| FoxM1 | Karsten, 2003 | NA | siRNA | Pos. regulator of cell cycle |
| B-MYB | | | SiRNA | Pos. regulator of cell cycle |
| | | | | (regulated by MELK) |

### Example 3

### Neural Stem Cell Self-Renewal Genes are Brain Tumor Hub Genes

Patterns of gene expression in brain tumors were studied and networks of genes that were interconnected were identified. This network can be subdivided into expression modules of related genes whose functions are similar. One of these modules was a cell cycle modules, genes that regulate the cell cycle. Within these networks were so-called “hub” genes—those whose expression were very highly interconnected with other members of the network. An analysis of hub genes and neural progenitor gene overlap was performed. Microarray data from mouse neural progenitor screens that were performed under the following conditions:
1 Spheres grown in bFGF vs. differentiating spheres.
2. Spheres Grown in bFGF vs. those grown in the EGFR ligand, TGF alpha
3. Spheres grown from PTEN-deficient vs. PTEN wildtype embryos.

In each of these samples, there was increased self renewal of neural stem cells under the first condition listed. When the list of 185 hub genes in the cell cycle module was examined, subtracting out those that were not represented on the microarray, approximately 40% of the hub genes that were present on the mouse arrays were enriched under conditions of enhanced stem cell self-renewal.

For example, genes were identified using microarrays from spheres grown in PTEN-deficient embryos versus PTEN wildtype embryos. Below is a list of genes that were enriched in PTEN knockout neurospheres and also found in the cell cycle expression module:

Gene Symbol

| | | |
|---|---|---|
| TOP2A | UBE2C | FEN1 |
| RACGAP1 | MELK | H2AFX |
| KIF4A | TOPK | STK6 |
| CDC2 | MCM7 | DNMT1 |
| EZH2 | CHAF1A | PCNA |
| HCAP-G | MCM6 | POLA |
| KIF2C | TMPO | TRIP13 |
| MAD2L1 | SPAG5 | MKI67 |
| NEK2 | BIRC5 | SLC35B1 |
| BUB1B | TYMS | |
| ECT2 | KPNA2 | |

### Example 4

### Genes that are Both Neural Stem Cell Genes and Glioma Hub Genes Regulate Brain Tumor Growth

Brain tumors contain cells that serve as cancer stem cells. That is, they were multipotent and self-renewing and, upon transplantation into immunodeficient mice, give rise to secondary tumors with the molecular and cellular characteristics of the parent tumors. See, e.g., Hemmati, supra (2003); Galli, R., et al., Cancer Res. (2004) 64:7011-7021; and Singh, S. K., et al., Nature (2004) 432:396-401. A remarkable degree of genetic overlap between cell cycle genes expressed by normal neural stem cell cultures and those that contain brain tumor stem cells has been identified. Genes likely to play key regulatory roles in glioma proliferation (so-called “hub” genes) were also highly associated with conditions of enhanced neural stem cell self-renewal. One of the genes found to be a “hub” gene, to be associated with brain tumor stem cells, and to regulate neural stem cell self-renewal was maternal embryonic leucine zipper kinase (MELK), a previously poorly characterized member of the AMPK/snf1 family. Such observations permit the identification of small molecule inhibitors of brain tumor cells, particularly those targeting MELK expression.

Small molecule inhibitors of MELK expression. MELK mRNA is highly expressed in brain tumors and brain tumor progenitors and correlates inversely with glioma outcome. Thus, inhibitors of MELK expression will regulate brain tumor stem cell growth.

Experimental Design Initial experiments utilize medulloblastoma (Daoy) and glioblastoma (U87) cell lines, both of which express MELK at a high level. Cells are infected with lentivirus containing the MELK promoter-EGFP sequence. The cells are sorted by FACS and plated at clonal density under growth conditions. Colonies are subcloned and expanded. Control lines are prepared using the CMV promoter.

We have developed a screen specifically for this purpose. The following exemplary protocol can be used: 1. Dispense MELK-EGFP or CMV-EGFP cells @ 10³-10⁵ cells in 384 well plates using a Multidrop 384 (Thermo LabSystems) and allow to attach overnight. 2. Compounds are added via pin-transfer of 50-100 mL of compound per well, resulting in an effective concentration of ˜10 uM. Compounds are provided by the MSSR and are from the ChemBridge DIVERset, a 30 k library of diverse small molecules. Plates are incubated for 24 hours at 37° C./5% CO₂ (or whatever). 3. Media is aspirated using a ELx405 plate washer, and cells are lysed for optimal quantitation of EGFP levels (automated microscopy can also be used to look at intact cells if needed). 4. EGFP fluorescence is quantified on an Analyst HT 384™ well plate reader (LJL Biosystems). CMV-EGFP cells will be used as a control (only hits specific for the MELK-EGFP are analyzed further).

Compound validation: Compounds that give “positive” (upregulation of MELK-EGFP) or “negative” (downregulation of MELK-EGFP) hits are added to the parent cell lines. MELK expression is assayed by RT-PCR after 24 hours. If the compounds regulate MELK, then their effects on multiple tumor cell lines and primary neural progenitor proliferation is determined using total cell number (as indicated by fluorescent vital dye staining) as well as BrdU incorporation. The most promising hits are resynthesized (to incorporate molecular tags, such as biotin) to facilitate target identification using affinity chromatography and (human) proteome microarrays.

Detection of compounds that inhibit the proliferation of or kill brain tumor stem cells. Putative brain tumor stem cells from gliomas and medulloblastomas were identified. See e.g., Hemmati, supra (2001). These cells can be highly enriched using FACS for the CD-133 antigen. See e.g., Galli, supra (2004); Singh, supra (2004). Compounds that selectively inhibit the growth of these cells are important lead compounds in the development of cancer stem cell-specific therapies.

Experimental Design Initial experiments are being carried out using cancer-derived progenitors using a fluorescent dye to estimate the number of living cells. 1,000 cells in a 384 well format are readily and reliably detected. For the screen, gliomas and medulloblastomas will be used. These studies will be repeated with the larger libraries. Cells will be sorted using anti-CD-133 (prominin) and 3000 positive cells will be placed into wells. Broad inclusion criteria will include those combinations of compounds yielding a 25% reduction in cell number after 3 days.

Compound validation: Compounds that are scored as “hits” are added to additional cultures of tumor-derived progenitors, primary neural progenitors and fibroblasts. Those compounds that specifically inhibit brain tumor progenitors or both brain tumor and primary neural progenitors, but not fibroblasts are pursued. Further analysis can include the determination of whether compounds influence normal neural stem cells as well as cancer stem cells and whether multiple types of brain tumor (and other tumor) cells are affected. The ideal candidate is one that has a broad range of antitumor activity, but which do not negatively influence normal stem cells.

### Example 5

### PBK/TOPK, a MAPKK Active During Neural Progenitor Mitosis

Previously, we performed genomic subtraction and gene expression profiling that identified the PDZ-binding kinase/TLAK cell originating protein kinase (PBK/TOPK) as a gene highly enriched in neural stem cell cultures. Here, with a combination of bioinformatic and experimental techniques, we show that PBK/TOPK, a MAPKK that is active in mitotic cells and phosphorylates P38 MAPK, plays an important role in neural progenitor proliferation. PBK/TOPK and P38 are activated in a cell-cycle dependant manner in neural progenitor cells, and this activation is necessary for their proliferation in vitro. In vivo, PBK/TOPK is expressed in cerebellar granule cell precursors from early post-natal animals, and in Mash1 positive, rapidly proliferating GFAP negative neural progenitors in the subependymal zone (SEZ). Using transgenic mice, we show that PBK/TOPK positive cells are GFAP negative, but arise from GFAP positive neural stem cells during adult neurogenesis, and are ablated concomitant with SEZ progenitor ablation, demonstrating that PBK/TOPK is a marker for transiently amplifying neural progenitors.

Neural stem cells (NSCs) are an endogenous, self-renewing population of cells capable of generating all major cell types of the mature central nervous system (CNS) (see, e.g., Capela, supra (2002); Lendahl, U., et al., Cell (1990) 60:585-595; Reynolds, B. A., et al., Science (1992) 255:1707-1710). NSCs exist throughout the germinal zones of the developing embryonic brain and persist into adulthood providing for ongoing neurogenesis in select regions of the mammalian brain, offering hope for neural repair strategies (see, e.g., Gage, supra (2000); Lie, D. C., et al., Annu. Rev. Pharmacol. Toxicol. (2004) 44:399-421; Morshead C. M., and van der Kooy, D., Curr. Opin. Neurobiol. (2004) 14:125-131; Palmer, T. D., et al., J. Neurosci. (1999) 8487-8497). In spite of their rich potential for therapeutic applications, research on NSCs has been hampered by a lack of markers to identify NSCs prospectively, and a rudimentary understanding of the intracellular pathways involved in the regulation of their proliferation and differentiation (see, e.g., Anderson, D. J., et al., Neuron (2001) 30:19-35; Lindvall, O., et al., Nat. Med. (2004) 10:S42-50). Notwithstanding this need, there has been progress in the area of marker identification and functional annotation, as evidenced by of genes such as Nestin (see, e.g., Lendahl, supra (1990)), EgfR (see, e.g., Doetsch, F., et al, Neuron (2002) 36:1021-1034), LeX (see, e.g., Capela, supra (2002)), NG2 (see, e.g., Aguirre, A. A., et al., J. Cell. Biol. (2004) 165:575-589), and the transcription factors DLX2 and MASH1, the latter of which is expressed in multipotent progenitors (see, e.g., Parras, C. M., et al., Embo. J. (2004) 23:4495-4505).

To identify genes involved in NSC proliferation and differentiation, we performed extensive analysis of the gene expression profile in primary neural stem cell cultures (see, e.g., Easterday, supra (2003); Geschwind, supra (2001); Karsten, supra (2003)). To provide further functional annotation that would aid in identifying genes involved in NSC proliferation or self-renewal, we identified genes expressed in multiple CNS germinal zones and non-neural stem cell populations (see, e.g., Easterday, supra (2003); Terskikh, supra (2001)). PDZ-binding kinase/TLAK cell originating protein kinase (PBK/TOPK) was one transcript found to be consistently elevated in all stem cell populations examined, even relative to other proliferating populations.

PBK/TOPK was not previously known to be involved in any facet of CNS development, but work in non-neural cells suggested that it was expressed in a variety of specialized, proliferative cell types. For example, PBK/TOPK expression was detected in male germ line progenitor cells, activated T-cells, and a variety of lymphomas and leukemias. However, it was not expressed in all highly-proliferative cell lines. For example, it was absent in WiDr and HT-29 colon cancer cells, indicating that it was not ubiquitously expressed in cycling cells (see, e.g., Abe, Y., et al., J. Biol. Chem. (2000) 275:21525-21531; Simons-Evelyn, M., et al., Blood Cells Mol. Dis. (2001) 27:825-829; Zhao, S., et al., Int. J. Biochem. Cell. Biol. (2001) 33:631-636). Previous work also suggested that PBK/TOPK was a cell-cycle regulated member of the MAPK Kinase family (see, e.g., Abe, supra (2000); Gaudet, S., et al., Proc. Natl. Acad. Sci. USA (2000) 97:5167-5172; Matsumoto, S., et al., Biochem. Biophys. Res. Commun. (2004) 325:997-1004). Activated PBK/TOPK phosphorylated P38 MAPK but not JNK or ERK MAPK in vitro or when overexpressed in COS-7 cells. Furthermore, activation of PBK/TOPK seemed to require phosphorylation by both the M-Phase kinase complex CyclinB/CDK1 and another unknown kinase, possibly RAFC or RAFA (see, e.g., Gaudet, supra (2000); Yuryev, A., et al., Genomics (2003) 81:112-125). These findings suggest that PBK/TOPK may play an important role in linking extracellular signals to intracellular state, possibly allowing extracellular influence on the cell cycle related processes of proliferation or differentiation. This is important to consider in the context of NSC, in which little is known about how cell cycle and cell fate mechanisms interact to allow a self-renewing pluripotent stem cell state.

Here, we investigated PBK/TOPK function and expression in the CNS. Initial functional annotation suggested a role for PBK/TOPK in the late phases of cell cycle for neural cells. Therefore, we tested the impact of manipulation of PBK/TOPK signaling on proliferation of cycling primary neuronal progenitors in vitro, confirming a role for PBK/TOPK in progenitor mitosis and proliferation. We then expanded these results to demonstrate PBK/TOPK expression in specific progenitor cells in vivo, showing by lineage mapping that PBK/TOPK is highly expressed in a critical population of neural progenitors: the transiently amplifying progenitor cell, and thus may serve as a useful marker for this population.

Results:

PBK/TOPK Transcript and Protein are Expressed Exclusively in Neurogenic Regions in Embryonic and Adult CNS:

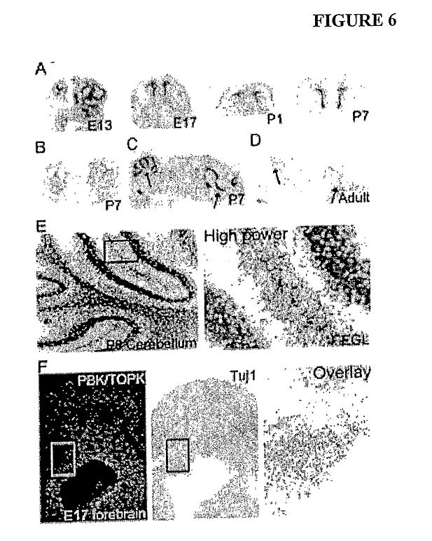

Previous array studies demonstrated that PBK/TOPK was expressed by a wide variety of stem and progenitor populations, consistent with a role in stem cell self-renewal (see, e.g., Easterday, supra (2003); Geschwind, supra (2001); Terskikh, supra (2001)). Here we verified continued expression of PBK/TOPK in postnatal regions of neurogenesis including the forebrain germinal zone, see FIG. 1A, developing hippocampus and dentate gyrus, see FIG. 6B, and the rostral migratory stream (RMS); see FIG. 6C, FIG. 6D. Especially striking was the expression in the external granular cell layer (EGL) of the cerebellum in the P7 animal, see FIG. 6E. At this age this structure is populated strictly by granular cell precursors, suggesting that PBK/TOPK is definitely expressed by neuronal progenitors. This is similar to PBK/TOPK expression in embryonic forebrain periventricular germinal zones, where it was expressed in the region containing stem and progenitor cells but not post-mitotic neurons, see FIG. 6F.

FIG. 6 illustrate data showing PBK/TOPK mRNA is specifically expressed in all germinal zones throughout neural development. FIG. 6A-D illustrate autoradiographic films of in situ hybridization with S³⁵ labeled PBK/TOPK antisense RNA in regions where stem and progenitor cells are found: FIG. 6A) PBK/TOPK is expressed in the forebrain germinal zones at E13 (sagital, whole head), E17 (coronal, whole head) P1 (coronal) and P7 (coronal) FIG. 6B) PBK/TOPK is also expressed in developing hippocampus and dentate gyrus at P7 (coronal) FIG. 6C) and in the developing cerebellum (red arrow) and the rostral migratory stream (RMS) (blue arrows) in sagital sections of P7. FIG. 6D) Expression continues in the subventricular zone and RMS of the sagital Adult brain (blue arrow), but not cerebellum (red arrow). FIG. 6E) Emulsion dipped and cresyl violet counterstained sections of the sagital P7 brain showing PBK/TOPK expression (black grains) in external granule layer, a region that only produces granule cell neurons. FIG. 6F) PBK/TOPK signal (black grains) does not overlap with immunoreactivity for immature neuronal marker Tuj1 (brown) in emulsion dipped coronal sections of E17 forebrain ventricular zone.

Preliminary Functional Annotation by Gene Co-Regulation Analysis: PBK/TOPK is Implicated in cell Cycle Progression.

For a bioinformatics based functional annotation of PBK/TOPK in neural cells, we used an approach based on the observation that genes that are involved in the same biological process are often tightly co-regulated (see, e.g., Eisen, M. B., et al., Proc. Natl. Acad. Sci. USA (1998) 95:14863-14868; Miki, R., et al., Proc. Natl. Acad. Sci. USA (2001) 98:2199-2204; Ren, B., et al., Science (2000) 290:2306-2309). If we identified specific categories of biological processes highly co-regulated with PBK/TOPK in nervous system tissue or cells, this would generate testable hypotheses regarding PBK/TOPK function in neural tissue. To perform this analysis, we needed a large neural dataset with variable PBK/TOPK expression, and so we capitalized on array data from neural tumors available in the NINDS/NIMH microarray database. Although brain tumors are not normal progenitors, they are proliferative, neural tissue, and data from several sources has recently demonstrated that some tumors, including gliomas, contain a multipotent tumor stem cell from which they derive (see, e.g., Galli, supra (2004); Hemmati, supra (2003); Ignatova, T. N., et al., Glia (2002) 39:193-206; Singh, S. K., et al., Cancer Res. (2003) 63:5821-5828). We reasoned that using this rich neural tumor dataset to derive hypotheses about the function of a neural stem cell gene would be appropriate, provided they these hypotheses were then tested in the non-cancerous, primary neural cells of interest. We therefore focused on a large scale study of gliomas in this database (see, e.g., Freije, W. A., et al., Cancer Res. (2004) 64:6503-6510). While PBK/TOPK was detected in 79 of the 85 gliomas in this set, it showed a high degree of variability, ranging over 50-fold, which made this data set amenable to assessing gene co-regulation.

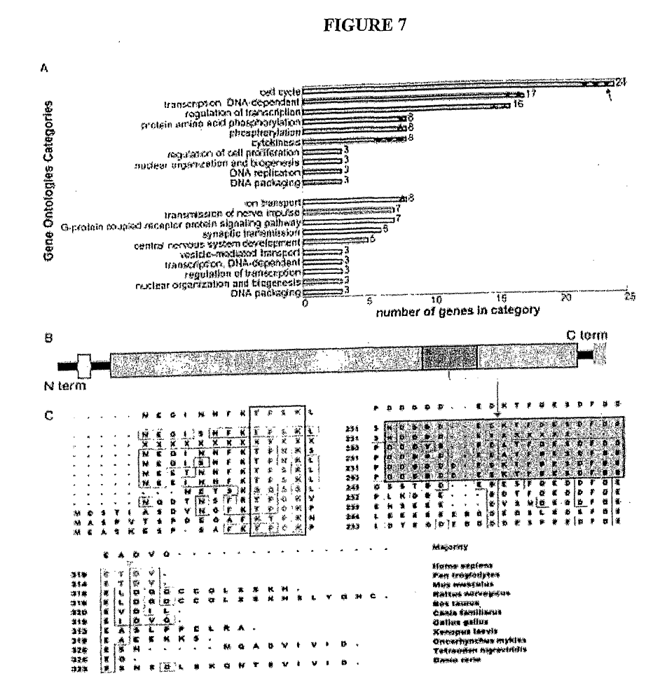

We created a gene co-regulation matrix and observed that PBK/TOPK was strikingly correlated with a large number of genes involved in the cell cycle machinery, including ki-67, aurora kinase B, and cyclin B1 (see Methods). To understand the level of significance of this finding in an unbiased manner, we performed statistical analysis of Gene Ontologies (G0) categories using EASE (see, e.g., Hosack, D. A., et al., Genome Biol. (2003) 4:R70), which revealed a significant overrepresentation of genes involved in the cell cycle (Ease statistic, p<10e-15), especially M-phase genes (Ease statistic, p<10e-12; see FIGS. 7A and 7B). This data in neural tissues was parallel to data showing cell cycle regulation of PBK/TOPK expression in synchronized HELA cells (see, e.g., Matsumoto, supra (2004); Whitfield, M. L., et al., Mol. Biol. Cell. (2002) 13:1977-2000). This specific pattern of correlation suggested that PBK/TOPK may play a fundamental role in the cell cycle in neural progenitor cells and tumors that derive from them, especially in M phase of the cell cycle. Furthermore, an examination of phylogenetic conservation of the PBK/TOPK protein sequence demonstrated a conserved cyclinB/CDK1 phosphorylation site and kinase domains, while the eponymous PDZ-binding motif is found only in primates, see FIG. 7C. Protein domains that are conserved across a variety of species may be important to a protein's function so we chose to focus our initial in vitro analysis on PBK/TOPK regulation in the cell cycle.

FIGS. 7A to 7C illustrate data showing that PBK/TOPK protein structure and expression in tumors suggests role in late cell cycle. FIG. 7A) PBK/TOPK was detectably expressed in 79 of 85 tumors by microarray analysis. To form hypothesis about the function of PBK/TOPK, we looked at the function of its correlates in this data set. Bars on graph represent functional classification by G0 biological process for top 100 PBK/TOPK-correlated (blue) and -anti-correlated (yellow) genes. 46 of 100 correlated genes and 21 of 100 anti-correlated genes were ‘known genes’ that could be categorized. Of these 46 genes, 24 were involved in the cell cycle (blue arrow). EASE was used to test for statistical overrepresentation of categories. Categories related to the cell cycle were the most significant for correlates, and processes related to ion transport were most significant for anti-correlates. *=p<0.05, **=p<0.005, ***=p<10e-10. FIG. 7B) Schematic of PBK/TOPK protein. Position of kinase domains is shown in gray, cyclinB/CDK1 phosphorylation site is shown in blue, aspartic acid rich region is shown in orange, and C terminal PDZ-binding motif in yellow. FIG. 7C) Multiple species alignment reveals conservation of cyclinB/CDK1 phosphorylation site, suggesting importance of this cell cycle motif. Sections of alignments for eleven species of vertebrates. Color-highlighted regions correspond to B. Boxes show matches to consensus. CyclinB/CDK1 site (T/S-P-X-K/R) is conserved in 10 of 11 species, but C-terminal PDZ-binding domain is not.

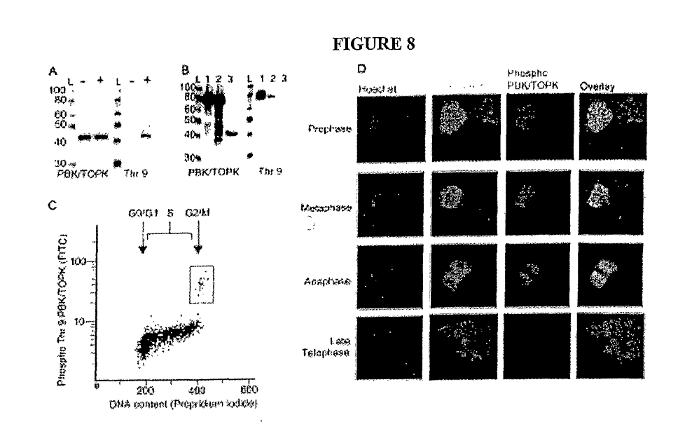

PBK/TOPK phosphorylation on cyclin B site is cell cycle regulated: The gene expression analysis of PBK/TOPK suggested an involvement in the cell cycle in neural cells, consistent with earlier work that suggest that PBK/TOPK was a cell cycle regulated kinase in non-neural cells, which required cyclinB/CD 1 phosphorylation for activation (see, e.g., Gaudet, supra (2000)). Therefore, we produced an antibody against a phosphorylated form of the cyclinB/CDK1 target site as a method of gauging activation of PBK/TOPK. This antibody recognized a PBK/TOPK sized band only in cells blocked in mitosis with nocodazole, see FIG. 8A. This antibody also detected recombinant, activated, GST-PBK/TOPK, and signal decreased dramatically when recombinant GST-PBK/TOPK was phosphatase-treated, see FIG. 8B.

We then examined the phosphorylation of PBK/TOPK across the cell cycle. Flow cytometry revealed that phospho-PBK/TOPK positive cells all had high DNA content indicating that they had likely finished S phase and were in either G2 or M phase of the cell cycle (FIG. 8C). Immunocytochemistry on N2a neuroblastoma, or P19 embryonic carcinoma cells revealed that phospho-PBK/TOPK was only detected in cells with condensed chromatin indicative of M-phase cells. Furthermore, it was clearly detected throughout all stages of mitosis, outlining condensed chromosomes, but its expression decreased abruptly and dramatically in telophase, and by late telophase PBK/TOPK protein itself seemed to disappear (FIG. 8D).

FIGS. 8A, 8B, 8C and 8D illustrate data showing phospho-PBK/TOPK expression is only detected during mitosis. FIG. 8A) An antibody raised against phosphorylated cyclinB/cdk1 site at Threonine #9 on PBK/TOPK only has signal in ME-180 cells treated with nocodazole, which blocks cells in mitosis (right blot). Probing with total PBK/TOPK antibody reveals equal amounts of PBK/TOPK protein in treated and untreated conditions (left blot). L=molecular weight marker, +=treated, −=untreated. FIG. 8B) PBK/TOPK (left blot) and Phospho-PBK/TOPK (right blot) antibodies recognize a recombinant activated GST-PBK/TOPK. Phospho-PBK/TOPK signal decreases with phosphatase treatment. L=molecular weight marker, 1=ProQinase active GST-PBK (80 kDa), 2=GST-PBK after lambda phosphatase treatment, 3=ME-180 (untreated) whole cell lysate. FIG. 8C) Flow cytometric analysis of untreated Jurkat cells, using Phospho-PBK/TOPK antibody labeled with FITC (Y-axis) versus DNA content measured by propidium iodide (X-axis), which can be used to measure position of a cell in the cell cycle. The boxed population indicates phospho-PBK/TOPK-positive cells have 4N DNA content indicative of G2 or M phase cells. FIG. 8D) 100× Immunocytochemistry on N2A cells shows phospho-PBK/TOPK is expressed specifically throughout mitosis, but expression of PBK/TOPK (green) and phospho-PBK/TOPK (red) decrease dramatically in late telophase. Notice that non-mitotic adjacent cells are phospho-PBK/TOPK negative.

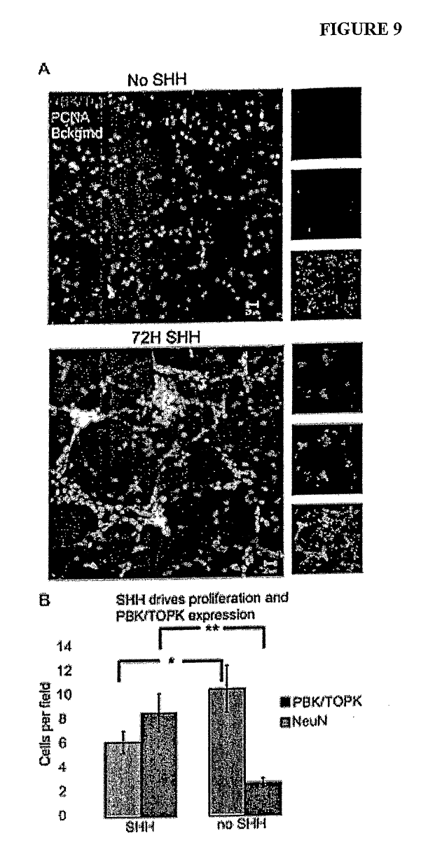

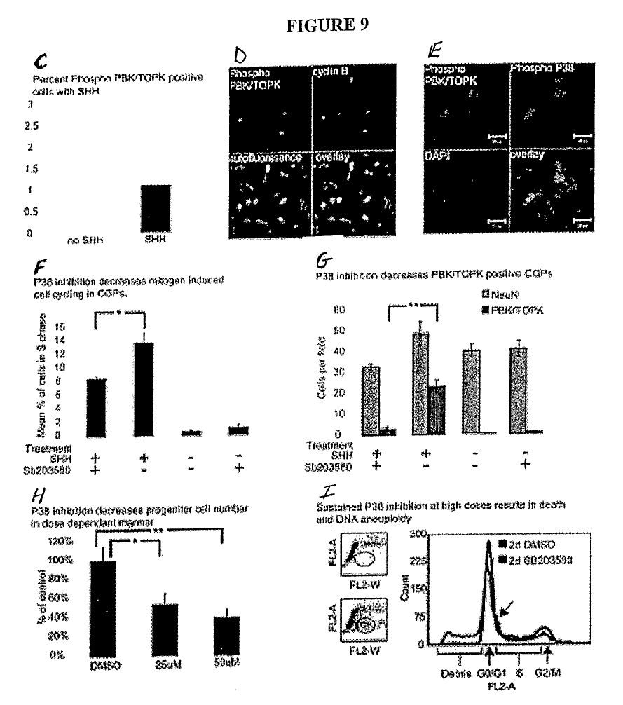

PBK/TOPK is expressed by proliferating cerebellar granule cell precursors: To confirm this finding in primary progenitors, we isolated primary cerebellar granule cells precursors (CGPs) from the EGL of the cerebellum, since PBK/TOPK was highly expressed in these cells, and they represent a relatively homogeneous progenitor pool, see FIG. 6E. Previous work has shown that CGPs will proliferate in vitro in response to the mitogen Sonic Hedge Hog (SHH) (see, e.g., Wechsler-Reya, R. J., et al., Neuron (1999) 22:103-114). We compared the expression of PCNA, PBK/TOPK, NeuN, and Doublecortin (Dcx) in CGP's cultured with or without mitogenic stimulus. PBK/TOPK expression overlapped highly with PCNA and expression of both PCNA and PBK/TOPK decreased dramatically in the absence of mitogen, see FIG. 9A, in which case CGPs developed elaborate Dcx positive process. In contrast, relative expression of NeuN, a marker of more mature neurons, increased in the absence of SHH, and PBK/TOPK expression did not overlap with NeuN, see FIG. 9B and FIG. 9C. This showed that in vitro PBK/TOPK is expressed in proliferating progenitor cells. Furthermore, as with N2a and P19 cell lines, phospho-PBK/TOPK was only detected in CGP's that were in M-phase, where it appeared to be highly associated with the mitotic apparatus, see FIG. 9D.

FIG. 9A through 9I illustrate data showing that PBK/TOPK is expressed by proliferating progenitors in vitro, and its activity is required for normal cell cycle. FIG. 9A) Consistent with what is seen in vivo in the cerebellum, 72 hours post-dissection, CGPs treated with mitogen SHH form proliferative clumps of PBK/TOPK, PCNA positive cells while the untreated cells stop proliferating and differentiate. FIG. 9B) Quantification of this effect reveals CGPs in cultures treated with mitogen maintain expression of PBK/TOPK. Untreated cultures have proportionally more cells that express the neuronal maturation marker NeuN. FIG. 9C) Strong P38 MAPK phosphorylation (red) occurs only in G2/M, cyclin B positive CGPs (green) in CGP suggesting P38 is activated primarily in G2/M phase in these cells. 9D) Strong Phospho-P38 positive (green) CGPs are always Phospho-PBK/TOPK cells (red) and vice versa, suggesting PBK/TOPK activates Phospho-P38 in this system. FIG. 9E) P38 MAPK specific inhibitor SB203580 reduces fraction of S-Phase cells in proliferating CGP. Harvested CGP were cultured with or without mitogen for 48 hours and 50 uM of the specific P38 MAPK inhibitor, SB203580 was added. 24 hours later, cells were harvested and subjected to cell cycle analysis via flow cytometric measurement of DNA content, and immunocytochemistry. FIG. 9F: described below. FIG. 9G) Cell counts of PBK/TOPK and NeuN positive cells reveals that SB203580 reduces PBK/TOPK positive cells, and total number of cells, without significantly impacting non-proliferative, differentiated NeuN positive cells. FIG. 9H) A similar effect is seen in multipotent neural progenitors cultured from E12 telencephalon, where there is a dose dependant decrease in proliferation as assessed by cell number. FIG. 9I) Forty-eight hours of exposure to highest dose of drug resulted in DNA aneuploidy. Dot plots: cell cycle assessment in progenitors as measured by measure of area (FL2-A) and width (FL2-W) of propidium iodide staining of DNA content. Yellow circle indicates normally cycling cells. Blue circle indicates cells with DNA aneuploidy, which have slightly more DNA (total area) than normal G1 cells but with unusual signal width. Histogram derived from dot plots shows a normal cell cycle profile and one treated with drug. DNA aneuploidy here is seen as the shoulder indicated by blue arrow. Note also increase in debris and decrease in S phase cells.

P38 MAPK is phosphorylated at mitosis in primary cerebellar granule precursor cells: In non-neural cells, PBK/TOPK phosphorylates P38 MAPK (see, e.g., Abe, supra (2000). Since MAPK pathways are highly conserved, we examined the phosphorylation of P38 MAPK in relation to proliferation and the cell cycle in primary CGP cells. We found that differentiation induced by mitogen withdrawal significantly reduced the number of cells positive for phospho-P38 (paired T-test, p<0.01). Furthermore, strong phospho-P38 was only detected in cyclin B positive CGP's (see FIG. 9C). Cyclin B is the G2/M phase-expressed cyclin that directs CDK1 to phosphorylate PBK/TOPK. Co-expression of cyclin B and phospho-P38 suggested that P38 is selectively activated in these cells in the G2/M phases of the cell cycle. Close examination of DNA in phospho-P38 positive cells revealed that they show the condensed chromatin characteristic of mitotic cells. Thus P38 appears to be phosphorylated specifically during mitosis in proliferating cerebellar granule cell precursors. This is consistent with other reports of mitotic activate on P38 in neuronal progenitors cultured from other germinal zones (see, e.g., Campos, C. B., et al., Neuroscience (2002) 112:583-591).

To determine whether PBK/TOPK was also active at this a time, we performed double labeling with phospho-PBK/TOPK and phospho-P38 MAPK. All phospho-PBK/TOPK positive cells were phospho-P38 positive, which strongly suggested that PBK/TOPK is responsible for phosphorylation of P38 MAPK in M phase in cerebellar granule cell precursors (FIG. 9D). The same results were seen in N2a and P19 cell lines.

Inhibition of the PBK/TOPK MAPKK signaling pathway decreases proliferation: Treatment of cultured primary CGP with the P38 inhibitor SB203580 (see, e.g., Gallagher, T. F., et al., Bioorg. Med. Chem. (1997) 5:49-64) resulted in a dramatic decrease in the fraction of cells in S-phase (FIG. 4E). Twenty-four hours of P38 inhibition caused a reduction in the number of the PBK/TOPK positive cells without any impact on the number of NeuN positive cells. However, in the absence of mitogen treatment on the primary progenitors, P38 inhibition had no impact on cell number (FIG. 9F), indicating that P38 inhibition was not killing cells in a non-specific manner, but specifically impacting the proliferating cells. To extend these findings to other primary progenitor populations in addition to the CGPs, we also exposed attached neural progenitors cultured from E12 mouse telencephalic germinal zones to varying doses of SB203580 in the presence of the mitogen bFGF. We saw a dose dependant decrease in cell number after exposure to drug (FIG. 9G), and a decrease in cycling cells as assessed by flow cytometry. At long exposures or high doses, we also observed an accumulation of DNA-aneuploid cells, suggesting that those cells that did survive were not completing normal divisions that accompany cell cycle progression (FIG. 9H), similar to what was recently seen in HELA cells treated with PBK/TOPK siRNA, where there was approximately a five fold increase in multinucleate cells (see, e.g., Matsumoto, supra (2004)).

PBK/TOPK is not expressed in post mitotic neuroblasts or mature glia in vivo: The in vitro data suggested a role for PBK/TOPK in neural progenitor cell cycle progression. To examine whether such a role was consistent with in vivo expression data, we performed a battery of immunohistochemical analyses with several markers of proliferation, differentiation, and progenitor states. Two different antibodies to PBK/TOPK in early postnatal and adult mice showed the same cytoplasmic and occasionally nuclear expression in the regions of ongoing neurogenesis: the sub-granular layer of the dentate gyrus, the subependymal zone (SEZ) of the lateral ventricles, and the rostral migratory stream (RMS) in all ages, and the EGL of the early postnatal cerebellum, consistent with in situ hybridization results.

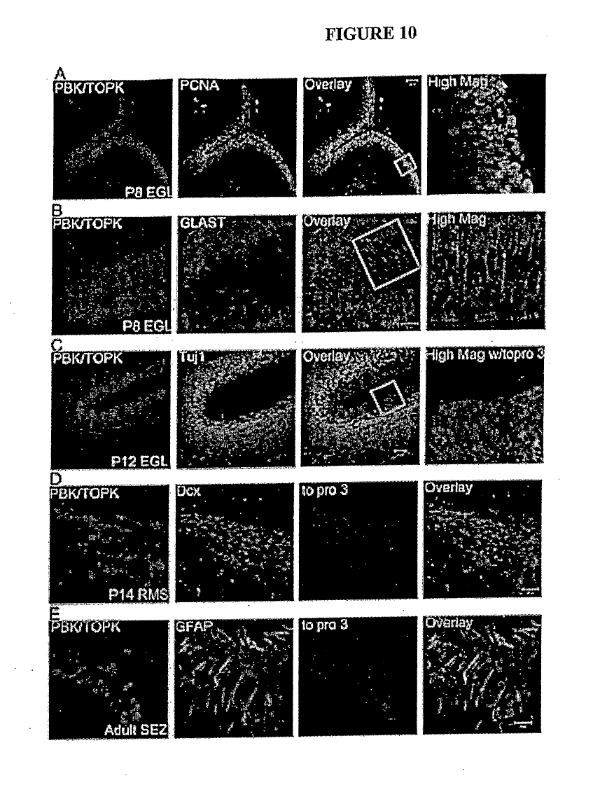

We examined the expression of PBK/TOPK in-depth in two regions of postnatal neurogenesis, the EGL, and the SEZ/RMS, to identify what cell type expresses PBK/TOPK. For the first two weeks after birth, cerebellar granule neurons continue to be born from the EGL. The EGL contains a mitotic layer with PCNA positive proliferating progenitors, a premigratory layer with post mitotic immature neurons, and the radially oriented processes of Bergmann glia (see, e.g., Migheli, A., et al, Am. J. Pathol. (1999) 155:365-373). Double-label immunohistochemistry of PBK/TOPK and Beta-III-tubulin (Tuj1) or Dcx (not shown), both markers of immature neurons, reveal clear expression of PBK/TOPK in the PCNA-positive mitotic (see FIG. 10A) but not the premigratory (see FIG. 10C) zones of the EGL. Not surprisingly, there was no PBK/TOPK expression in the cerebellum in the P21 or adult animal, when neurogenesis in the cerebellum is complete (not shown). In the EGL, scaffolding for cell division and migration are provided by GLAST positive Bergmann Glia (see, e.g., Furuta, A., et al., J. Neurosci. (1997) 17:8363-8375), which are PBK/TOPK negative (FIG. 10B). This pattern of expression is consistent with a role in proliferation of neuronal progenitors in the cerebellum.

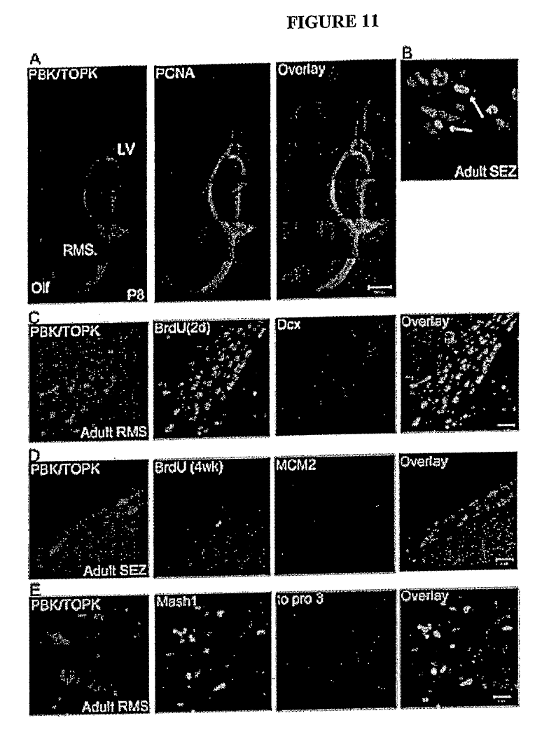

In adult animals, PBK/TOPK is expressed sporadically within the subgranular layer of the dentate gyrus and strongly within the subependymal zone of the lateral ventricle. Especially striking is the postnatal PBK/TOPK expression in the neurogenic SEZ and the full extent of the rostral migratory stream from the anterior lateral ventricle to the beginning of the olfactory bulb (see FIG. 11A). This expression is seen in all ages examined, but decreases in intensity from P7 to adulthood. Most PBK/TOPK positive cells were also PCNA positive when examined at high magnification. To further assess PBK/TOPK expression within migratory immature neuroblasts in the SEZ and RMS, we performed double labeling with Tuj1 and Dcx. PBK/TOPK was expressed adjacent to, but not in, clusters of Dcx (see FIG. 10D) positive cells, consistent again with PBK/TOPK being expressed in proliferating neuronal progenitors, but not in post-mitotic immature neurons.

Recent evidence strongly suggests that the primary stem cell of the adult central nervous system is GFAP positive (see, e.g., Doetsch, F., et al., Cell (1999a) 97:703-716; Garcia, A. D., et al., Nat. Neurosci. (2004); Imura, supra, (2003)). We performed double label immunohistochemistry for GFAP and PBK/TOPK to assess potential co-expression. We did not observe any double-labeled cells in the adult subependymal zone (see FIG. 10E), suggesting if they did exist, they were relatively rare.

FIGS. 10A, 10B, 10C, 10D and 10E illustrate data showing PBK/TOPK protein is not expressed in neurons or mature glia in EGL or the SEZ and RMS. FIG. 10A) PBK/TOPK (red) is expressed in cytoplasm of cells in Proliferating Cell Nuclear Antigen positive (PCNA—green) mitotic layer of P8 EGL. Right panel: 100× magnification of region similar to box. FIG. 10B) PBK/TOPK (red) is not expressed in GLAST (green) positive Bergmann Glia whose fibers provide scaffolding in P8 EGL. Right panel: 100× magnification of region in box. FIG. 10C) PBK/TOPK (red) is not expressed in Tuj1 (green) positive immature granule cell neurons in P12 EGL. Right panel: 100× magnification of region similar to box, with topro-3-iodide (blue) added to label nuclei. FIG. 10D) PBK/TOPK (red) does not overlap with immature migrating neurons expressing Dcx (green) in a sagital postnatal RMS. Nuclei counterstained with topro-3-iodide (blue). FIG. 10E) PBK/TOPK (red) does not generally overlap with GFAP (green) positive mature astrocytes in adult SEZ counterstained with topro-3-iodide (blue). All scale bars 20 uM.

PBK/TOPK expression overlaps with markers of proliferation and progenitor cells: To further examine the cellular context of PBK/TOPK expression, we performed double and triple label immunohistochemistry with neural progenitor cell markers and several markers of cell proliferation (see FIG. 10A, FIG. 11A-D). After 4 injections of BrdU over two days, all PBK/TOPK positive cells were BrdU positive (see FIG. 11B). However, there were many BrdU positive, but PBK/TOPK negative cells. Most of these cells were Dcx positive. We surmised that the PBK/TOPK BrdU double positive cells were currently proliferating population of cells, while the Dcx/BrdU positive population represented primarily recently born neurons.

We tested this hypothesis in more detail, first, by co-labeling with the PCNA antibody, which recognizes a subunit of the DNA-polymerase III complex involved in DNA synthesis during S-Phase (see e.g., Tsurimoto, T., Front. Biosci. (1999) 4:D849-858). Virtually all (>90%) of the cells expressing PBK/TOPK in all regions, and at all ages, also expressed PCNA, suggesting that PBK/TOPK was expressed in cycling cells (see FIG. 10A, FIG. 11A). However, not all of the PCNA positive cells were PBK/TOPK positive, suggesting that the majority (>90%), but not all cycling cells in this region expressed PBK/TOPK (see FIG. 11B). Next, we examined whether PBK/TOPK was expressed by rapidly cycling cells, or slowly cycling cells, by assessing PBK/TOPK expression several weeks after BrdU injections. This typical experimental design is based on the observation that slower cycling cells retain BrdU, while rapidly cycling cells dilute the BRDU beyond detection. The MCM2 protein, which is expressed in G1 phase, was used to determine whether any of the slowly cycling cells were re-entering the cell cycle (see e.g., Maslov, A. Y., et al., Neurosci. (2004) 24:1726-1733). Four weeks after BrdU injection, we saw few BrdU positive cells in the SEZ and did not see any BrdU/MCM2 double positive cells, suggesting the BrdU labeled cells were quiescent or post mitotic. There was no overlap between PBK/TOPK and BrdU positive cells, suggesting PBK/TOPK is not expressed in SEZ cells when they are not currently proliferative. There was, however, extensive overlap generally between MCM2 and PBK/TOPK, once again demonstrating that PBK/TOPK is expressed in actively cycling cells (see FIG. 11D).

To examine PBK/TOPK expression in putative neuronal progenitors outside the EGL, we studied its co-localization with the proneural bHLH transcription factor Mash1. Mash1 has recently been shown to specify neuronal and oligodendritic fate in the postnatal brain in vitro and in vivo (see e.g., Parras, supra (2004)) and Mash1 knockout mice have morphological defects of the olfactory bulb (see e.g., Guillemot, F., Biol. Cell (1995) 84:3-6; Murray, R. C., et al., J. Neurosci. (2003) 23:1769-1780; Parras, supra (2004)). This suggests that Mash1 may play a role in olfactory bulb neurogenesis, which occurs throughout a mammal's lifetime, as neurons born in the subependymal zone of the anterior lateral ventricle and rostral migratory stream (RMS) migrate to the olfactory bulb (see e.g., Luskin, M. B., Neuron (1993) 11:173-189). We reasoned that if Mash1 plays a role in neurogenesis in the embryonic olfactory bulb, it may also play a role in adult SEZ neurogenesis. Consistent with this, we observed Mash1 expression for the extent of the SEZ and RMS in the adult animal, as was recently reported by Parras, supra (2004). Strikingly at least 90% of Mash1 positive cells in adult SEZ were also clearly PBK/TOPK positive, suggesting that PBK/TOPK is expressed in a significant proportion of neuronal progenitors destined for the olfactory bulb (see FIG. 8E). Mash1 and PBK/TOPK double positive cells were seen in SEZ and RMS, but the PBK/TOPK positive cells of the EGL of the cerebellum at P7 were clearly Mash1 negative, demonstrating that not all neurogenic cells expressed Mash1, and not all PBK/TOPK cells were Mash1 positive. However, at least 70% of PBK/TOPK positive cells were Mash1 positive in adult SEZ, showing that PBK/TOPK is highly enriched in these progenitors.

FIGS. 11A, 11B, 11C, 11D and 11E illustrate data showing PBK/TOPK expressed exclusively in rapidly proliferating progenitor cells in postnatal rodent brain. A) Montage of 15 10× fields showing front third of sagitally cut P8 brain. PBK/TOPK is expressed for the extent of subependymal zone (SEZ) of the lateral ventricle and RMS around PCNA positive nuclei. LV=lateral ventricle, RMS=rostral migratory stream, Olf=olfactory bulb. B) Most PBK/TOPK positive cells have PCNA positive nuclei: 100× section of adult SEZ showing PCNA single labeled (blue arrow) and PCNA-PBK/TOPK double labeled (yellow arrows) cells. C) In adult RMS after 4 BrdU pulses in 2 days, all PBK/TOPK (red) cells are BrdU (green) positive, showing recent birth or current proliferation. Remaining BrdU positive cells overlap with Dcx (blue), a marker of immature neurons, but no clear overlap of Dcx and PBK/TOPK was detected. D) 4 weeks after a BrdU injection, there is still extensive overlap between proliferation marker MCM2 (blue) and PBK/TOPK (red) and but no overlap with MCM2 or PBK/TOPK and BrdU (green) in what are presumably slower cycling and quiescent cells. E) There is extensive overlap between Mash1 positive (green) neuronal progenitor nuclei and PBK/TOPK (red) in adult RMS.

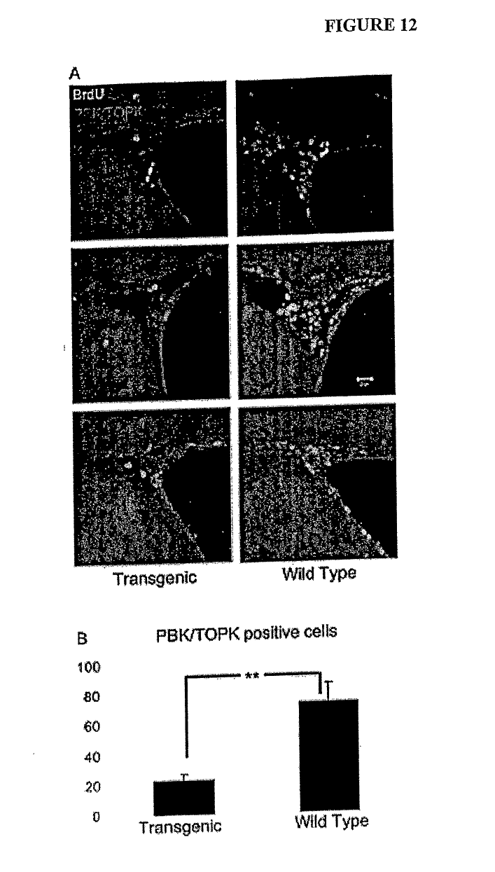

PBK/TOPK positive cells are mitotically active progenitors in vivo: The expression pattern in vitro as a mitotically active kinase, coupled with in vivo data, provided evidence that PBK/TOPK was expressed in several mitotically active progenitor cell populations in the central nervous system, but not in quiescent, slower cycling putative stem cells. To more conclusively establish PBK/TOPK expression in stem and progenitor cells in vivo, we examined PBK/TOPK in transgenic mice expressing the herpes simplex virus thymidine kinase gene (HSV-TK) under control of the GFAP promoter (see, e.g., Bush, T. G., et al., Neuron (1999) 23:297-308; Garcia, supra (2004)). Since HSV-TK phosphorylates the drug ganciclovir and phosphorylated ganciclovir kills cells at mitosis, when these mice are given ganciclovir, all dividing GFAP positive cells are killed. Previous work has shown that when these mice are treated with ganciclovir, there is a complete ablation of stem cell potential, as assessed by multipotent neurosphere forming ability in vitro (see, e.g., Imura, supra (2003); Morshead, C. M, et al., Eur. J. Neurosci. (2003) 18:76-84), and a complete ablation of neurogenesis in vivo (Garcia, supra (2004)). Three transgenic mice were treated for 21 days with ganciclovir followed by BrdU injections for two days, and then processed for immunohistochemistry. This treatment resulted in a complete ablation of Dcx positive cells in SEZ, and a roughly 70% reduction in PBK/TOPK positive cells (Independent Samples T-Test, p<0.001) in the SEZ, relative to non-transgenic controls. All remaining PBK/TOPK positive cells were BrdU positive, demonstrating that they had been born after the discontinuation of ganciclovir treatment (see FIG. 12A, 12B), or were cycling cells not yet killed by the ganciclovir, which is lethal at mitosis. This finding provided direct in vivo evidence that PBK/TOPK positive cells arise from proliferating GFAP positive cells, consistent with PBK/TOPK being expressed exclusively by proliferating progenitor cells in the adult brain.

This finding raised several important questions, including an apparent paradox: ablation of GFAP positive cells lead to ablation of PBK/TOPK positive cells yet, at any given point in time, we were unable to identify any cells expressing both GFAP and PBK/TOPK. This suggested a lineage relationship, such that TOPK/PBK positive cells are generated from GFAP positive cells, but represent a different stage in progenitor lineage. Based on the previous data presented, this would be consistent with the PBK/TOPK positive cells being expressed by a rapidly proliferating, GFAP negative, population of cells, called transient amplifying cells, that arise from more quiescent, GFAP positive cells, as suggested by, e.g., Alvarez Bullya, Doetsch, and others (see, e.g., Doetsch, supra (1999a); Doetsch, F., et al., J. Neurosci. (1997) 17:5046-5061; Doetsch, F., et al., Proc. Natl. Acad. Sci. USA (1999b) 96:11619-11624; Morshead, C. M., et al., Development (1998) 125:2251-2261; Morshead, supra (2003); Morshead, C. M., et al., Neuron (1994) 13:1071-1082).

FIGS. 12A and 12B illustrate data showing PBK/TOPK cells were dramatically reduced when stem cells are ablated. FIG. 12A) Subependymal zones from three replicate wild type (bottom) and transgenic animals (top) treated with 21 days of ganciclovir to ablate neurogenesis, and then given BrdU (green), show clear reduction of PBK/TOPK positive cells (red). FIG. 12B) Quantification using STEREOINVESTIGATOR™ reveals a highly significant 70% decrease in PBK/TOPK positive cells in transgenic animals.

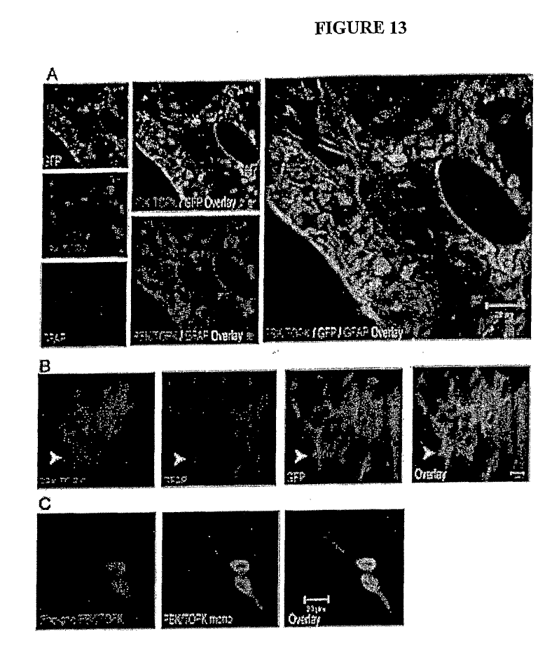

To test this hypothesis, we utilized a transgenic mouse that was generated by crossing a GFAP promoter driven CRE, with a floxed-stop-eGFP reporter (Garcia, supra (2004)). In this mouse, all progeny of cells that have ever expressed GFAP become permanently eGFP positive. Virtually all (>95%) of the PBK/TOPK positive cells in SEZ were eGFP positive, demonstrating conclusively that they arise from GFAP positive cells (see FIG. 8A), even thought they are themselves GFAP negative. In one mouse, we were also able to detect one PBK/TOPK positive cell containing a GFAP positive fiber (see FIG. 13B). This cell had the condensed chromatin indicative of a mitotic, metaphase cell (not shown), perhaps suggesting that some proliferating GFAP positive cells may express PBK/TOPK at mitosis. This would be consistent with our difficulty in detecting GFAP PBK/TOPK double positive cells: if only 10% of GFAP positive cells in the SEZ are proliferating (Garcia, supra (2004)), and about 1% of proliferating cells are mitotic, this would make such PBK/TOPK-GFAP positive these cells rare in vivo. These data are further supportive of the general model, discussed below, which contains a transient amplifying (C cell) in adult SVZ neurogenesis (Doetsch, supra (1997)).

FIGS. 13A, 13B and 13C illustrate data showing PBK/TOPK cells are GFAP negative progeny of GFAP positive cells; FIG. 13A) Subependymal zone from a transgenic where all progeny of GFAP positive cells express eGFP. Virtually all PBK/TOPK positive cells (red) are GFAP negative (blue), but are progeny of GFAP positive cells (green). FIG. 13B) A rare SEZ cell containing a GFAP (blue) fiber (white arrow) expressed PBK/TOPK when undergoing mitosis. FIG. 13C) Astrocytes cultured from postnatal forebrain also express PBK/TOPK (green) and phospho PBK/TOPK (red) during mitosis.

Discussion: We previously initiated studies to discover and characterize genes that could serve as potential markers of neuronal progenitors, or provide functional insight into neuronal progenitor proliferation, by implementing a gene expression profiling strategy that employed representational difference analysis subtraction coupled to microarray screening (see, e.g., Geschwind, supra (2001)). We further stratified that profile with extensive screening for overlapping expression in stem cell populations and germinal zones, identifying a few key genes with enriched expression in multiple stem cell populations, suggesting a role in progenitor self-renewal and proliferation (see, e.g., Easterday, supra (2003)). Here, we have extensively characterized one of those candidates: PBK/TOPK, a MAPKK not previously known to be involved in CNS development. We show, using in vitro and in vivo models, that PBK/TOPK plays an important role in the proliferation of progenitor populations, and serves to identify a specific population of proliferating progenitors in the adult brain, the transient amplifying cell (see, e.g., Doetsch, supra (1997)).

Analysis of gene co-regulation networks in a large-scale microarray data set supported the hypothesis that PBK/TOPK was a highly regulated protein, whose function was most likely related to M-phase in the cell cycle. Study of its sequence conservation across phylogeny from zebrafish to human supported this by identifying a highly conserved putative cyclin B phosphorylation site, again suggesting a role in mitosis. So, we explored this hypothesis in vitro by examining cell cycle regulation of PBK/TOPK using a phospho-specific antibody directed against the thr-9 site that comprised the likely target of CyclinB/CDK1. In both cell lines and primary neuronal progenitors from the cerebellum, we demonstrate that this site is specifically phosphorylated only in mitosis and that this phosphorylation rapidly disappears in late telophase. Further, P38 MAPK, PBK/TOPK's target in non-neural tissue (see, e.g., Abe, supra (2000), is also phosphorylated at mitosis in neural cells: P38 phosphorylation is observed only in cyclinB positive cells, with condensed chromatin, and its appearance is enhanced by mitogen. Finally, addition of the P38 MAPK inhibitor sb203580 resulted in a significant decrease in the number of cells re-entering S-phase and large degree of DNA aneuploidy. There was both a decrease in the total number of cells and the number of PBK/TOPK positive cells, without a significant impact on the number of non-proliferating NeuN positive cells. This data demonstrates that PBK/TOPK signaling is essential for the proliferation of neuronal progenitors in vitro.

In vivo, the current studies also provide clear evidence that neuronal progenitors, and possibly multipotent progenitors, express PBK/TOPK while they are proliferating. First, PBK/TOPK is strongly expressed in the mitotic layer of the external granule layer, in PCNA positive, Dcx, Tuj1, and GLAST negative cells. This structure only gives rise to cerebellar granule neurons, thus PBK/TOPK must be expressed in the precursors of these neurons. This is supported by data showing that purified CGP in vitro express PBK/TOPK, which is mitogen dependent, parallel with the SSH requirement for CGP proliferation (see, e.g., Wechsler-Reya, supra (1999)). Indeed, in culture, PBK/TOPK expression still overlaps strongly with PCNA, a marker of proliferation, and not with NeuN, a marker of maturing neurons. Furthermore, many proliferating cells in the SEZ and RMS, structures that give rise to neurons throughout the life of the animal (see, e.g., Luskin, supra (1993)), are positive for both PBK/TOPK and the pro-neural Mash1 gene. Considered together, all of the evidence clearly demonstrates PBK/TOPK is expressed by multiple neuronal progenitor populations during development, and possibly multipotent progenitors as well (see, e.g., Parras, supra (2004)).

Cytological investigations have identified three major morphologically-defined populations of cells in the SEZ: ultrastructurally migrating neurons, GFAP positive glial cells, and highly proliferative ultrastructurally immature cells (see, e.g., Doetsch, supra (1997); Doetsch, supra (2002)). The current belief is that slow cycling GFAP positive cells give rise to rapidly cycling GFAP negative progenitor cells which then give rise to immature neurons that migrate to the olfactory bulb. This model has been supported by studies that demonstrate neurospheres must directly or indirectly arise from slow cycling and GFAP positive cells (see, e.g., Doetsch, supra (1999a); Garcia, supra (2004); Imura, supra (2003); Morshead, supra (1998); Morshead, supra (1994)). Using three different markers of proliferation, PCNA, MCM2, and short pulse BrdU labeling, we show that PBK/TOPK cells are a highly proliferative population of cells. Using markers of migrating immature neurons, Tuj1 (see, e.g., Menezes, J. R., et al., Mol. Cell. Neurosci. (1995) 6:496-508) and Dcx, we have shown that PBK/TOPK expressing cells are adjacent to but not overlapping with these immature neurons. Neither was there generally detectable overlap with quiescent GFAP positive slow cycling cells in adult animals.

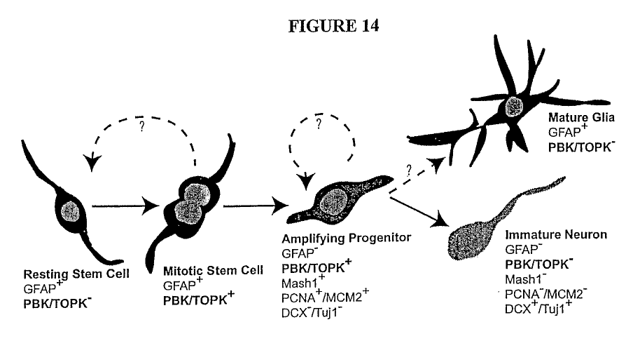

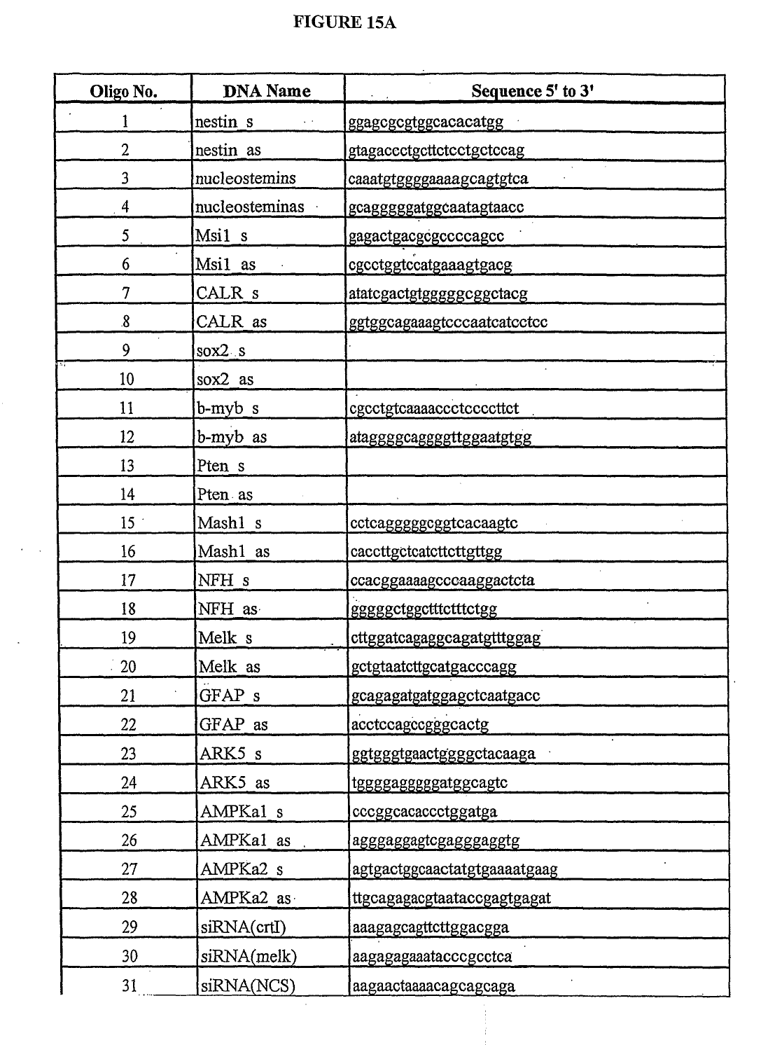

This data demonstrate the following model for PBK/TOPK expression in the adult SEZ, illustrated in FIG. 14: A resting GFAP positive cell in the SEZ enters the cell cycle. PBK/TOPK is expressed by mitosis of this GFAP positive cell, and continues to be expressed by rapidly proliferating MASH1 positive, and GFAP negative progeny. These cells then give rise to PBK/TOPK negative, Tuj1, Dcx positive immature neuronal progeny that migrate to the olfactory bulb to become mature neurons. This model is strongly supported by the dramatic decrease in PBK/TOPK positive cell number following ablation of GFAP positive cycling cells, and the lineage experiments that demonstrate that PBK/TOPK positive cells must arise from GFAP positive cells. Therefore, the mitotic kinase PBK/TOPK is likely to serve as a marker of transiently amplifying progenitor cells in the SEZ and provides further support for the models proposed by Alvarez-Buylla, Deutsch and colleagues (Doetsch, supra (1997); Doetsch, supra (2002).

FIG. 14 shows a model of PBK/TOPK expression in adult neurogenesis: past evidence and our current studies suggest that there is a quiescent population of GFAP positive, PBK/TOPK negative stem cells (blue) in the adult subependymal zone that can be recruited to the cell cycle. These cells are express PBK/TOPK during mitosis and divide either symmetrically or asymmetrically to give rise to at least one, PBK/TOPK positive, GFAP negative, rapidly proliferating cell (red), that in turn, gives rise to PBK/TOPK negative, DCX positive post-mitotic immature neurons (green). Relative amount of amplification of rapidly proliferating progenitor cell is unknown, and it is also unknown if progeny of PBK/TOPK positive cells can become glia. This diagram only includes markers investigated here.

While we have demonstrated that PBK/TOPK can serve as marker for this distinct class of progenitor cells in the adult.

These data have identified PBK/TOPK as significantly enriched in this critical mitotically active population, its regulated phosphorylation during this process, and its relationship to p38 MAPK provides another tool with which to begin to understand molecular pathways of cell cycle regulation and their coupling to cell fate decisions in the CNS (Anderson, supra (2001); Ohnuma, S., et al, Neuron (2003) 40:199-208).

Experimental Procedures

In situ hybridization: In situ hybridization was performed as previously described Geschwind, supra (2001). Probes from a 384 bp fragment (Genbank CA782113), and full length PBK/TOPK had identical expression patterns. In situ/immunohistochemistry double labeling was done as described (see, e.g., Kornblum, H. I., et al., Eur. J. Neurosci. (1999) 11:3236-3246). For all in situ hybridizations, sense RNA controls showed no labeling above background.

Analysis of microarray data: Data were downloaded from NINDS/NIMH database (arrayconsortium.tgen.org). This set included 85 gliomas from 79 patients hybridized onto Affymetrix HG133A & B arrays (see, e.g., Freije, supra (2004)). Arrays were normalized with dCHIP (www.dchip.org) and expression values were calculated (see, e.g., Li, C., et al., Proc. Natl. Acad. Sci. USA (2001) 98:31-36). We filtered for genes with a coefficient of variation>0.8 to identify genes that varied significantly across samples. After filtering, there were 2,217 probes representing 1,874 highly variable genes, including PBK/TOPK. Pearson's correlation was performed to identify genes whose expression co-varied at a highly significant level across the samples. DAVID was used to classify correlated and anti-correlated genes into level 5 Biological Processes Gene Ontologies (see, e.g., Dennis, G., Jr., et al, Genome Biol. (2003) 4:P3). EASE was used to test for statistical overrepresentation of categories relative to a background of all 1,874 analyzed genes (see, e.g., Hosack, supra (2003). Results are similar if less filtered or unfiltered gene sets are used, or if either whole array or whole genome is used for background comparison in EASE analysis.

Phylogenic analysis: Complete PBK/TOPK sequence for Homo sapiens and Mus musculus have already been reported (see, e.g., Abe, supra (2000); Gaudet, supra (2000); Zhao, supra (2001). To identify key functional domains, we searched EST libraries with NCBI's BLAST, and draft genome sequences with UCSC's Blat for PBK/TOPK sequence in all available vertebrate species. From these sequences, we were able to construct complete putative homologues for PBK/TOPK in Rattus norvegicus, Xenopus laevis, Gallus gallus, Danio rerio, Oncorhynchus mykiss, Canis familiarus, Tetraodon negroviridis and nearly complete sequences for Pan troglodytes and Bos taurus using the DNASTAR Seqman software. We aligned these sequences with clustalW, implemented on DNAstars Megalign software.

Culture of Cerebellar Granule Cell Precursors: Cerebella were harvested from P6-P8 CD1 mouse pups and digested in Papain with DNase, and dissociated in PBS BSA with fire polished pipettes followed by a cell strainer. Granule cell precursors were then separated on a 35%/65% percoll step gradient at 1500 g for 12 minutes as previously described (Wechsler-Reya, supra (1999)). Cells were either transfected at this point or plated at 250K cells/well onto Poly-L-Lysine coated glass coverslips in 24 well plates in 330 uls of Neurobasal Media containing 2% B27 supplement, 1 mM sodium pyruvate, 2 mM glutamine, and 1% penicillin/streptomycin, supplemented with 2.5 ug/ml mouse recombinant Sonic Hedge Hog (R&D systems 461-5H-025) as noted in the text, above.

Flow cytometry for cell cycle: Staining was as described: (see, e.g., Krishan, A., J. Cell. Biol. (1975) 66:188-193). Briefly, cells were lysed in a hypotonic, buffer with Triton X-100, RNase, and Propidium iodide. DNA content of >13,000 nuclei was measured with using CELLQUEST software and a Facscaliber cytometer from Becton Dickinson, and data were analyzed with ModFit 3.1 with service pack 2 for Macintosh to determine percentages of cells. All C.V.'s and RCS values were below five.

Preparation and specificity of anti-PBK/TOPK antibodies: The phosphospecific (Thr9) and total PBK/TOPK antibodies were produced by immunizing New Zealand White rabbits with synthetic peptides. The following peptides, coupled to keyhole limpet hemocyanin, were used: Thr9(P) (NFKT*PSKLSEKC) (SEQ ID NO:2) and total PBK/TOPK (CTNEDPKDRPSAAHIVE) (SEQ ID NO:3). Immunoglobulin was purified using protein A-Sepharose. To ensure phosphospecificity of the phospho-PBK/TOPK (Thr9) antibody, antibodies reactive with the nonphosphopeptide were removed by adsorption to a nonphosphopeptide affinity column. Antibodies that flowed through this column were passed over a column of immobilized phosphopeptide; after the column was washed, antibodies were eluted at low pH and dialyzed. For total PBK/TOPK, protein A-Sepharose purified antibodies reactive with the immunogenic peptide column were eluted and dialyzed. Analysis of the phosphospecificity of the resulting phospho-Thr9 antibody and phospho-independence of the resulting total PBK/TOPK antibody was performed by immunoblotting against (i) whole-cell extracts from control and nocodazole-blocked ME-180 cells and (ii) recombinant activated GST-PBK/TOPK or the protein dephosphorylated in vitro with lambda phosphatase (NEB#P0753). The phospho-independence of the total PBK/TOPK antibody was further established by comparing whole cell extracts from NIH-3T3 and PC12 cells that were treated with the Ser/Thr phosphatase inhibitor calyculin A (CST #9902) to extracts that were subjected to in vitro dephosphorylation with lambda protein phosphatase.

Animals: Transgenic mice were created and treated as described (Bush, supra (1999); Garcia, supra (2004); Imura, supra (2003)).

Immunohistochemistry: Post-natal day 7 (P7), P12, P14, P21 CD1, and adult CD1, C57/BL6, and transgenic mice were perfused trans-cardially with ice cold PBS followed by ice cold 4% paraformaldhyde in PBS, pH 7.4. Brains were removed, fixed in 4% paraformaldhyde overnight, sunken 20% sucrose PBS, frozen in 4-methyl-butane, and stored at −80° C. until use. Forty micron sections were cut on a cryostat and stored in PBS 0.1% azide at 4° C. until use. Free floating sections were incubated overnight in 24 well plates on a rotator at room temperature in the presence of 0.1% azide, 0.25% triton and 5% normal goat serum in 500 uls PBS and primary antibody at the following concentrations: anti-PBK/TOPK serum 1:500 (Gaudet, supra (2000)), anti-PBK monoclonal 1:50 (BD Transducin 612170), anti-Beta III Tubulin (Tuj1) 1:1000 (Covance MMS-435P), anti-Mash1 1:20 (556604 BD Pharmingen), anti-GLAST 1:5000 (Chemicon AB1782), anti-Doublecortin 1:500 (Chemicon AB5910), anti-PCNA 1:10000 (DakoCytomation M 0879), anti-BrdU 1:5000 (Maine Biotech PAB 105P), anti-GFAP 1:1000 (Chemicon AB 1540). For BrdU and PCNA, antigens were retrieved by incubating sections 1 hour at 65 C in 50% formamide, 2×SSX, and 30 minutes in 2.0 N HCl at 37° C. Secondary antibodies were diluted 1:1000 and included cy2, cy3, and cy5 conjugated antibodies (Jackson Immunoresearch) and Alexa 350, 488, 568, 594 conjugated antibodies (Molecular Probes). For some antibodies (monoclonal PBK/TOPK) signal was sometimes amplified with Tyrimide Signal Amplification. In all cases, no primary controls yielded no labeling except in P7 animals anti-mouse IGG alexa 488 apparently labels some cells with a glial morphology. Where necessary, subtype specific antibodies were used to avoid this confound.

Nuclei were counterstained with DAPI-containing mounting media (Vector Labs) or with Topro-3-iodide (Molecular Probes), a nuclear stain fluorescing in the far red range (650 nm), by exposing tissue sections for 5 min to a 20 micromolar solution in PBS.

Immunocytochemistry: Coverslips were harvested and fixed in 4% paraformaldhyde, washed in PBS, and blocked for 30 min in 5% NGS 0.25% triton PBS. Cells were then exposed to primary overnight at room temperature at the following concentrations anti-PBK/TOPK 1:500 (serum) or 1:100 (monoclonal), NeuN 1:250 (Chemicon MAB377), PCNA 1:5000, Doublecortin 1:500, Cyclin B1:500 (Cell Signaling Technology 4125), Phospho-P38 monoclonal 1:100 (Cell Signaling Technology 9216) Phospho-P38 polyclonal 1:500 (Cell Signaling Technology 9211). Secondaries and counterstaining were as above.

Microscopy: All fluorescent images were acquired on either a Leica TCS-SP MP Confocal and Multiphoton Inverted Microscope (Heidelberg, Germany) and a two photon laser setup (Spectra-Physics) or Zeiss LSM 510 META confocal microscope, using lasers and filters appropriate for the fluorophores, and pseudo-colored images were overlaid with Zeiss software or Adobe Photoshop. Infrared wavelengths were most often pseudo-colored blue.

Cell counts: For immunocytochemistry, all cells in 10 random 40× fields were counted. For the stem cell ablation experiments we used stereo investigator to count all PBK/TOPK cells in the SEZ ventral to the anterior horn at the level of the anterior commissure bilaterally in 3 replicate animals. T-tests were performed in Microsoft Excel.

### Example 6

### MELK can Inhibit the Growth of Brain Tumor Cells In Vivo

The following example illustrated that practicing the methods and compositions of the invention using the exemplary MELK-inhibitory compositions can inhibit the growth of brain tumor cells in vivo.

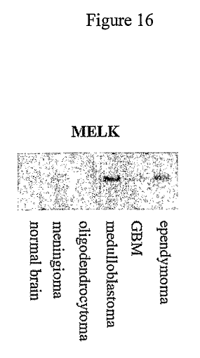

MELK is expressed by brain tumors and roughly correlates with grade: Our studies with neural progenitors led us to begin investigation of MELK in brain tumors. Initial, semi-quantitative RT-PCR analysis, using GAPDH to normalize, demonstrated that MELK mRNA was expressed in several different kinds of brain tumors, see FIG. 16, with a suggestion of higher expression in medulloblastoma, ependymoma (the sample shown here is an anaplastic ependymoma) and lower expression in oligodendroglioma, meningioma and normal brain. FIG. 16 illustrates data from an RT-PCR analysis of brain tumor and normal brain samples.

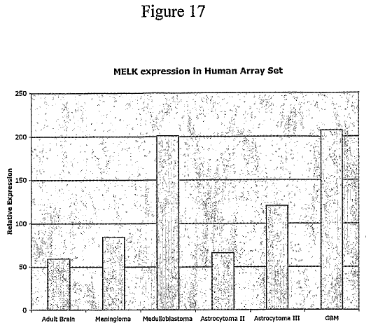

To more systematically analyze MELK expression in brain tumors, we examined expression of MELK across several tissue samples that have been hybridized to the Affymetrix U133™ microarray. Data were analyzed and normalized to a global mean for each array and presented here as the mean of each subtype. In this data set were approximately 73 GBMs, 5 medulloblastomas, 8 grade III and 2 grade II astrocytomas. Highest levels of expression were found in medulloblastomas and GBM, with lower levels of expression in low grade tumors, see FIG. 17, illustrating data showing normalized MELK expression levels in a variety of tumor types and normal brain. There is poorer survival amongst GBM patients with higher levels of MELK expression than those with lower level of MELK expression.

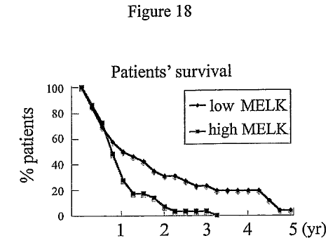

In a large GBM microarray dataset, it was calculated that the relative risk of MELK expression for death to be 1.75, suggesting that patients with higher levels of MELK expression do more poorly than those with lower levels. To further examine this, we divided patients up into 2 groups; the upper 50% and lower 50% as determined by MELK expression on the microarray. As shown in FIG. 18, the survival curve of the higher expressers diverged from those of the lower expressers. These data indicate that higher levels of MELK expression are found in more aggressive tumors. FIG. 18 illustrates a survival curve of patients with GBM divided into two groups; high vs. lower MELK expression.

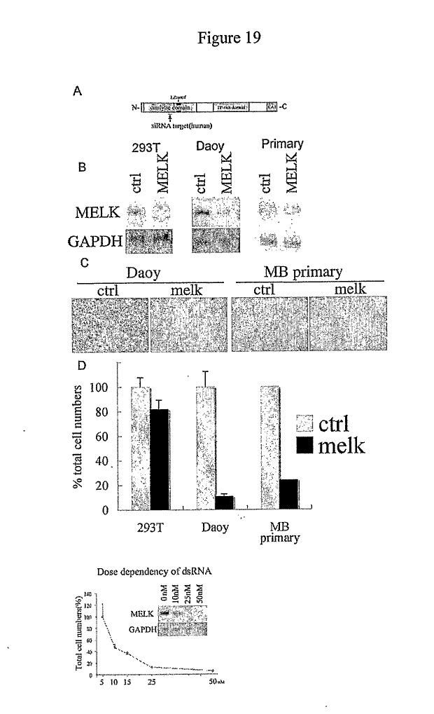

MELK regulates medulloblastoma cell proliferation and/or survival. We have demonstrated that MELK mRNA is expressed in the cerebellar EGL, the probable cells of origin of medulloblastomas. Therefore, we tested the effects of MELK siRNA to influence the proliferation and/or survival of Daoy and primary medulloblastoma cells in vitro. As shown in FIG. 19, MELK siRNA dramatically inhibited the growth of medulloblastomas, while there was little or no effect on 293T cells. FIG. 19 illustrates data demonstrating the results of human medulloblastoma cells treated with RNAi for MELK in culture. (FIG. 19A) A schema showing targeting of siRNA. A different region of human MELK is selected compared with mouse target. (FIG. 19B) MELK expression is downregulated by siRNA treatment of three human cell types. 293T, fibroblast cell line; Daoy, medulloblastoma cell line; and MB primary tumor culture from a patient with medulloblastoma. (FIG. 19C) Pictures of siRNA treated Daoy cells and MB primary cells after siRNA treatment (a) and the graph indicates resultant total cell numbers (b). (FIG. 19D) Total cell numbers following treatment with MELK siRNA. The dose-dependency is shown at the bottom.

This latter observation further demonstrates that MELK does not regulate the proliferation of all dividing cells. In the Daoy cells, it is likely that MELK influences cell survival in addition to proliferation as there is a dramatic effect on cell number, even after only 2 days of treatment. Additionally, we observed an increased number of condensed and fragmented nuclei, consistent with the hypothesis that knockdown of MELK induces apoptosis.

Low expression was also found in oligodendrogliomas and Schwannomas. Expression was high in one anaplastic ependymoma and lower in a lower grade ependymoma. While these data have limitations in the numbers of samples from different tissue types, they are strongly suggestive that MELK expression correlates with the tumor grade in gliomas and that MELK is highly expressed in medulloblastomas. It is interesting to note that low grade gliomas do not have higher levels of expression of MELK than normal brain. It is possible that MELK does not play a role in the proliferation of these tumors, or that MELK is only expressed by a minority of cells, such as the tumor stem cells. The analysis of array data also suggest that MELK is not simply a marker of proliferation, since some highly proliferative tumors that are not of neural tube origin have low levels of MELK expression. For example, even high grade meningiomas had the same low level of MELK expression as low grade meningiomas, with a mean relative expression of 80 vs. 87, respectively. It is important to note that MELK is not uniquely expressed in tumors of neural origin. Single samples of metastatic lung and colon cancer had very high levels of MELK expression.

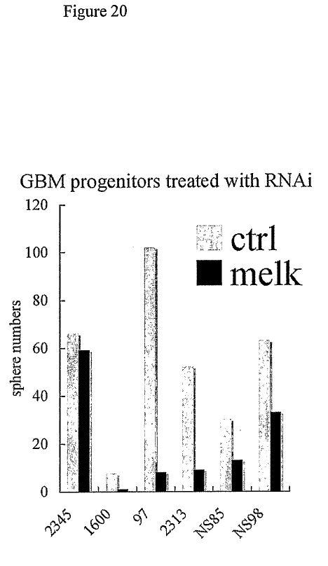

In order to determine whether MELK influences the proliferation of GBM cells, we examined the effects of MELK knockdown on the proliferation of primary cultures derived from GBMs. Tumor samples had been previously dissociated and plated in monolayer cultures until confluent. After thawing, the cultures were grown on substrate in serum and then treated with MELK or siRNA and transferred into sphere-forming conditions (see, e.g., Hemmati et al., 2004) to assay for the production of putative brain tumor progenitors. The number of spheres were counted one week following treatment. As shown in FIG. 20, MELK siRNA inhibited sphere production in 5/6 gliomas, indicating that it may play an important role in the production of brain tumor stem cells. Data illustrated in the graph of FIG. 20: GBM progenitors were treated with siRNA, and grown as spheres; the graph shows the numbers of spheres after siRNA treatment of each patient's sample.

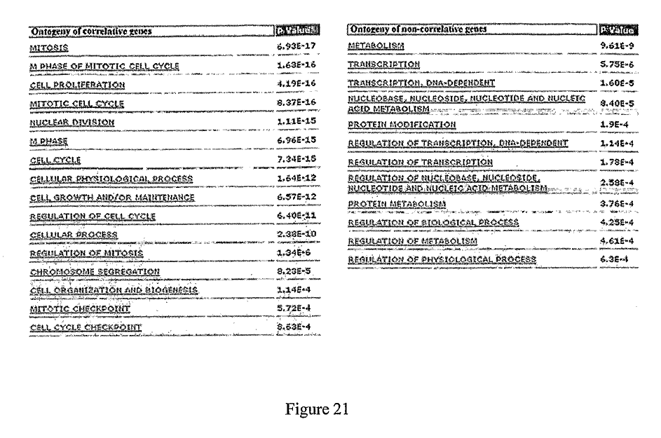

MELK expression in gliomas is co-regulated with other cell cycle genes: Our functional studies demonstrated a role for MELK in glioma proliferation, but do not demonstrate the mechanisms. To explore some of these potential mechanisms, we took advantage of a large microarray data set derived from human brain tumors (see, e.g., Freije et al., 2004) to identify genes whose expressions were co-regulated with MELK. Microarray data from each tumor was normalized to the global mean and a pairwise comparison made between MELK and each gene. The Pearson coefficient of correlation was then determined and a ranked list developed. There were 1,601 genes were positively correlated with MELK expression and 1,317 anti-correlated. The general functional category of each positively and negatively correlated was then determined using DAVID/EASE (http://apps1.niaid.nih.gov/david). As shown in FIG. 21, (from Nakano et al., 2005), MELK expression was highly and significantly correlated with genes known to play roles in the cell cycle, especially those associated with the mitosis (M) phase. FIG. 21 illustrates data showing gene ontology of MELK correlated genes (left) and anti-correlated genes (right). The p-value is the degree of significance of the positive and negative correlations for each group. Genes whose expression was not correlated with MELK functioned in other processes as determined by Gene Ontology, including metabolism, transcription and protein modification. Thus, this genome-wide analysis of co-regulation supports a role for MELK in cell cycle regulation. It's high correlation with genes that promote cell cycle progression suggests, but does not prove, that MELK serves a similar function, rather than an inhibitory role.

Examination of the identity of MELK-associated genes revealed that many of the most correlated genes had been previously identified as part of the cell cycle gene co-expression module, with the genes most highly correlated with MELK expression being more likely to be part of the cell cycle co-expression module than those not quite as highly correlated. Of these genes identified, all were associated with a poor prognosis (relative risk significantly greater than 1), see Table 2:

TABLE 2
GBM genes associated and unassociated
(anti-correlated) with MELK.

| | Associated | | Unassociated |
|---|---|---|---|
| | Top 10 most | Top 100 most | (100 most |
| | associated | associated | anti- |
| | genes | genes | correlated) |
| Fraction present in the cell | 5/10 | 19/100 | 0/100 |
| cycle module | | | |
| Fraction of the genes listed | 5/5 | 19/19 | N/A |
| above that are associated | | | |
| with poor prognosis | | | |

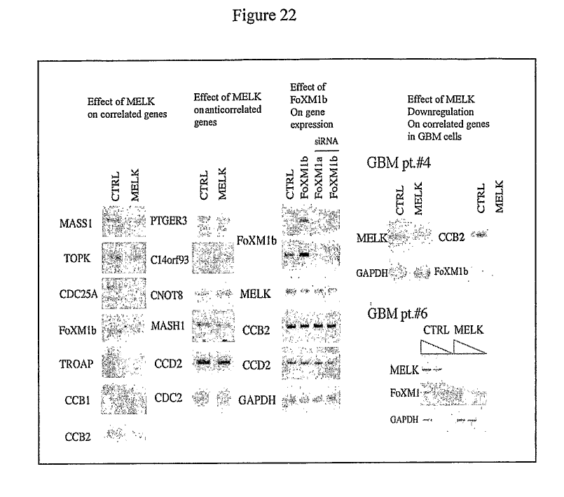

These observations raised the possibility that MELK either regulates or is part of a pathway that is important for GBM proliferation and malignancy. To demonstrate this hypothesis, we examined the effects of MELK knockdown on the expression of genes that were correlated or anti-correlated with MELK in Daoy cells, see FIG. 22. These cells were used for our initial studies, as we find siRNA knockdown to be highly reliable and reproducible in these cultures. Preliminary data indicate that 48 hr following treatment with the MELK siRNA the expression of 6/7 of the genes correlated with MELK examined was markedly diminished, as revealed by RT-PCR. However, none of the anti-correlated genes were downregulated by treatment with MELK siRNA, with some genes appearing to increase in expression. Additionally, we performed similar experiments in a few primary GBM samples. MELK siRNA treatment caused a knockdown of cyclin B2 and FoXM1 in one GBM sample, and FoXM1b in another (the only genes assayed), confirming that similar mechanisms are likely to hold in Daoy and GBM cells. These data demonstrate that MELK regulates the expression of the genes that are co-regulated with it. Knockdown of FoXM1b, however, did not influence MELK expression, suggesting that MELK is upstream of FoXM1 or that the loss of FoxM1b expression does not reduce the survival of MELK-expressing cells.

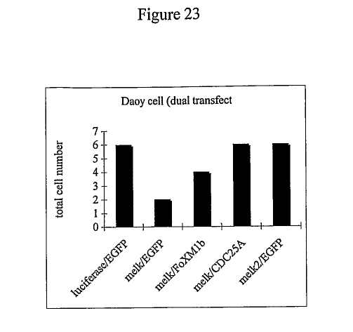

FIG. 23 illustrates gene expression in Daoy cells treated with either MELK or Luciferase (ctrl) siRNA. On the left is the effect of MELK siRNA on MELK-associated genes, in the middle is the effect of MELK siRNA on expression of MELK-unassociated genes. Gene expression investigated by RT-PCR in 2 GBM samples demonstrating knockdown of expression of cyclin B2 and FoXM1.

In order to further elucidate the MELK pathway, we performed a rescue experiment. FoXM1, CyclinB2 and CDC 25 are all part of the FoXM1 pathway that regulates the cell cycle (see, e.g., Teh et al., 2002). Daoy cells were treated simultaneously with MELK or control (luciferase) siRNAs along with a FoXM1b (the active form), CDC25 or EGFP (control) overexpression vector. As shown in FIG. 23, our observations indicated that FoXM1 and CDC25A are capable of at least partial rescue of the reduced cell number seen in MELK siRNA-treated cells. Effects of MELK siRNA are rescued by FoXM1b. Daoy cells were treated with MELK siRNA and co-treated with EGFP (control), FoXM1, or CDC25A cDNAs. The MELK2 siRNA is inactive.

These data are consistent with the hypothesis that MELK regulates its activity through the FoXM1 pathway, although other explanations are certainly possible. MELK regulates the growth of brain tumor cells following implantation.

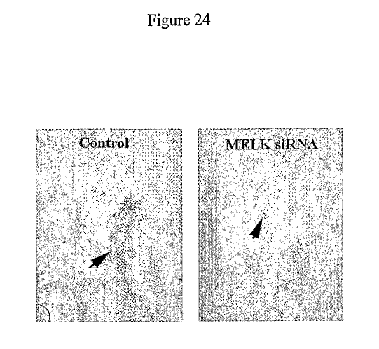

We also examined the role of MELK in experimental brain tumors in vivo. We incubated progenitors with MELK or control siRNA immediately prior to implantation into immunodeficient mice (N=3). After two weeks, the brains were fixed, sectioned and stained using an antibody directed against human nuclei. As shown in FIG. 24, we found virtually no cells on the experimental (MELK knockdown) side while a mass of cells was present on the control side. These data demonstrate that MELK knockdown inhibits brain tumor progenitor growth in vivo. FIG. 24 illustrates data demonstrating that MELK siRNA treatment ex vivo inhibits in vivo growth of ependymoma progenitors. Cells were treated with MELK siRNA and then transplanted into immunodeficient mice. Brains were removed after 2 weeks and then stained for anti-human nuclear antibody. Similar results were obtained in 3 separate animals. Arrows show implanted cells.

### Example 7

### Inhibition of Maternal Embryonic Leucine Zipper Kinase (MELK) Diminishes Self-Renewal of Multipotent Progenitors Derived from the Cortex

This example presents studies demonstrating that MELK is expressed in subsets of progenitors in the developing and adult brain and can serve as a marker for self renewing multipotent neural progenitors in embryonic and postnatal cortical cultures. Overexpression of MELK enhances while inhibition (knockdown), using, e.g., small interfering RNA (siRNA), diminishes self-renewal of multipotent progenitors derived from the cortex. This function is likely to be mediated by the proto-oncogene B-myb and independent of the PTEN controlled signaling/akt pathway. MELK is upregulated during the transition of neonatal GFAP-positive astrocytes to LeX-positive rapidly amplifying progenitors in vitro, and MELK downregulation by siRNA treatment dramatically inhibits this transition. These data demonstrate that MELK is a critical regulator of the proliferation of self-renewing multipotent progenitor cells from the embryonic brain and that it regulates the transition from type B stem cells to type C rapid amplifying cells in the postnatal brain.

Self-renewal and multipotency are critical properties of stem cells. This is certainly the case with neural stem cells which are defined by their ability to self-renew, and their capacity to produce the three major cell types of the brain: neurons, astrocytes and oligodendrocytes (see, e.g., Gage, 2000; Momma et al., 2000; Panchision and McKay, 2002). In the adult subventricular zone (SVZ), type B cells, a slowly dividing glial fibrillary acidic protein (GFAP)-positive cell type, are thought to be neural stem cells; while type C cells, a more rapidly proliferative population of self-renewing multipotent progenitors, are derived from the type B cells (for review, see Garcia-Verdugo et al., 1998; Alvarez-Buylla et al., 2002). In early brain development, it is not clear whether such distinctions exist. Therefore, we will use the term multipotent progenitor cell (MPC) to generally denote self-renewing, tripotent cells, derived from the CNS.

In this study, we tested the hypothesis that MELK is expressed by multipotent neural progenitors and regulates their proliferation. Expression analysis revealed MELK to be highly enriched in neural progenitors in vitro and in vivo. Double labeling of developing brain sections using in situ hybridization and immunohistochemistry demonstrated that MELK is expressed specifically in PCNA-positive proliferating progenitor cells in the brain but not ubiquitously in proliferating cells outside of the brain. We also demonstrated that cultured MELK-expressing cells were co-localized with neural progenitor markers, LeX, and nestin by immunocytochemistry, while MELK was not expressed by cells bearing markers of differentiation or lineage commitment. Analysis of MELK function demonstrated that MELK regulates MPC self-renewal, and the underlying signaling mechanism was independent of PTEN/AKT pathway and likely mediated through the protooncogene, B-myb.

Additionally, we found that MELK expression was increased as GFAP-positive astrocytes progressed to GFAP-negative, LeX-positive progenitors with the competence to produce neurons in neonatal cortical progenitors. Inhibition of MELK expression during this process resulted in a dramatic decrease in the appearance of LeX-positive cells without significantly influencing cell death.

In summary, these data demonstrated that MELK-regulates MPC self-renewal through regulation of MPC cell cycle in the embryonic brain. Furthermore, MELK regulates the transition of GFAP-expressing neonatal astrocytes to a GFAP-negative rapidly amplifying progenitor state in vitro.

MELK is expressed by neural progenitors: In our previous study, we identified MELK to be enriched in cortical (bFGF or TGF alpha-stimulated) or striatal neurospheres (NS) derived from P0 mice, as compared to cells that had been differentiated for 24 hours—conditions under which the MPC population decreased by 10-fold. We also found MELK to be highly expressed in hematopoietic stem cells.

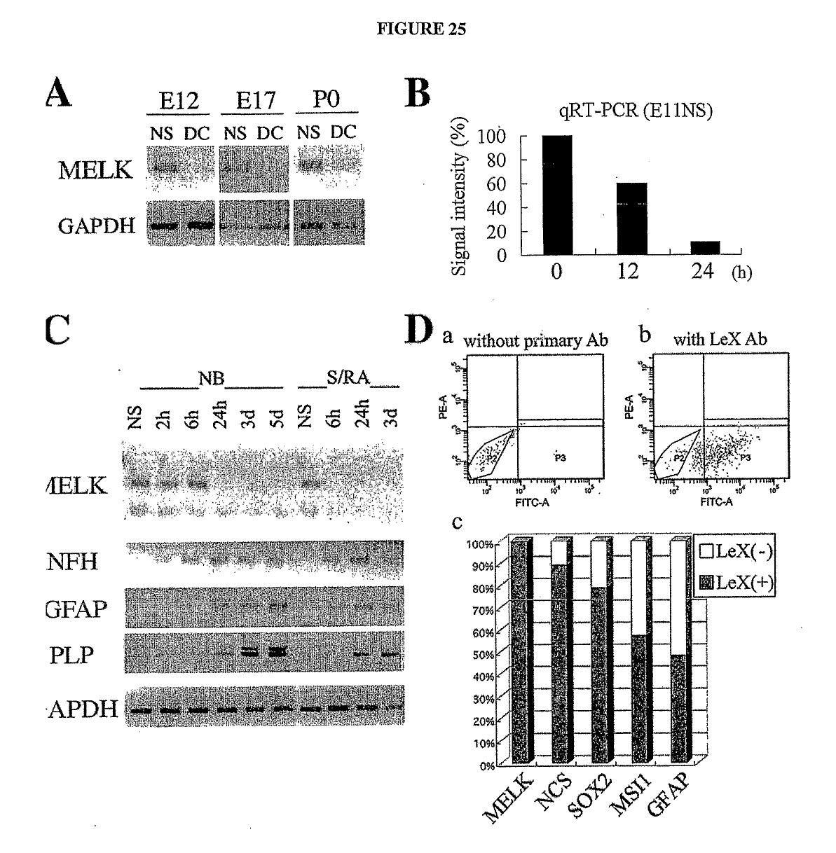

Since MPC have different characteristics, depending upon the age at which they are isolated, we further analyzed MELK expression in neurospheres derived from the following tissues: E12 telencephalon, a stage of neurogenesis; E17 cortex, a transitional stage; and P0 cortex, a time of gliogenesis. The results shown in FIG. 25A demonstrate that MELK was expressed by each NS population and downregulated after mitogen withdrawal. MELK was also expressed in NS derived from adult striatal subventricular zone. To quantify the enrichment of MELK expression in E11 NS, real-time RT-PCR was used. MELK mRNA levels declined to 60% within 12 hours of bFGF withdrawal in neural progenitors and to less than 10% of the original expression after 24 hours (see FIG. 25B).

In order to further determine the characteristics of cultures under differentiation conditions and to provide a basis for subsequent studies, we used NS derived from E12 brains and differentiated them by two methods: withdrawal of bFGF and addition of fetal bovine serum and retinoic acid (see FIG. 25C). The differentiation of the cultures was confirmed by increased expression of neurofilament heavy chain (NFH), GFAP, and proteolipid protein (PLP), marker of neuronal, astrocytic and oligodendroglial differentiation, respectively. Under both conditions, MELK expression declined with the onset of expression of differentiation markers.

To further determine the association of MELK with neural progenitors, we tested whether MELK mRNA was present in cells that express LeX/SSEA1. This cell surface molecule is known to be expressed by and enriched in multipotent, self-renewing MPC from brain or NS cultures. Cortical NS cultures from embryos at E12 were attached to polyornithine-fibronectin substrate and then LeX-positive and LeX-negative cells were separated by FACS sorting using an anti-LeX antibody (see FIG. 25D). Approximately 65 percent of the cells in the cultures were LeX-positive (FIGS. 25D, a and b). RT-PCR analysis demonstrated that MELK mRNA was completely restricted to the LeX-positive fraction, with no detectable expression in the LeX-negative fraction (FIG. 25D, c shows the relative signal between LeX positive and negative cells). LeX-sorting also yielded an enrichment for other stem cell-associated genes, including nucleostemin (NCB), SOX2, and musashi-1 (Msi1). Both NCS and SOX2 were highly expressed in LeX-positive populations. In contrast, GFAP was more highly expressed in the LeX-negative fraction, although it was still expressed in the LeX-positive fraction, as described previously (Capela and Temple, 2002), consistent with a status as a “marker” for both differentiated astrocytes and some multipotent progenitors. Taken together, these findings indicate that MELK is enriched in multipotent neural progenitor populations from embryonic brain.

FIG. 25 illustrates data showing that MELK is highly enriched, in multiple neural stem cell-containing cultures. FIG. 25A. MELK expression is higher in undifferentiated neurospheres compared to differentiated cells. Neurospheres were isolated from brains of the ages shown (NS), and one half of the cells were incubated in the absence of added mitogen to induce differentiation (DC). The cells were then subjected to semiquantitative RT-PCR, using GAPDH as a standard. Note the higher expression level in the NS compared to DC. FIG. 25. Quantitative RT-PCR shows that MELK expression in E11 cortical neurosphere cultures declines over ten-folds following withdrawal of bFGF within 24 hours. FIG. 25C. MELK expression declines with differentiation induced by mitogen withdrawal or stimulation of retinoic acid and fetal bovine serum. E12 neurospheres (NS) were differentiated and then subjected to RT-PCR analysis at various times later for MELK and for lineage-specific markers: neurofilament (NF) for neurons, glial fibrillary acidic protein (GFAP) for astrocytes and proteolipid protein (PLP) for oligodendrocytes. Under both differentiation conditions, the decline in expression of MELK is associated with upregulation of those three lineage markers. FIG. 25D. MELK expression segregates with neural progenitor marker, LeX. Anti-LeX antibody was used for cell sorting of neural progenitors from E12 telencephalon. 65% of the sorted cells were LeX positive (P3 in b), and 35% were LeX negative (P2 in b). Less than 0.2% of cells showed false-positivity, when incubated in the absence of primary antibody (P3 in a). RT-PCR demonstrates that LeX positive cells have stronger expression of several stem cell markers, including SOX2 and nucleostemin (NCS), while GFAP expression was more enriched in LeX negative cells (c). Transcripts of MELK are exclusively detected in LeX positive fractions.

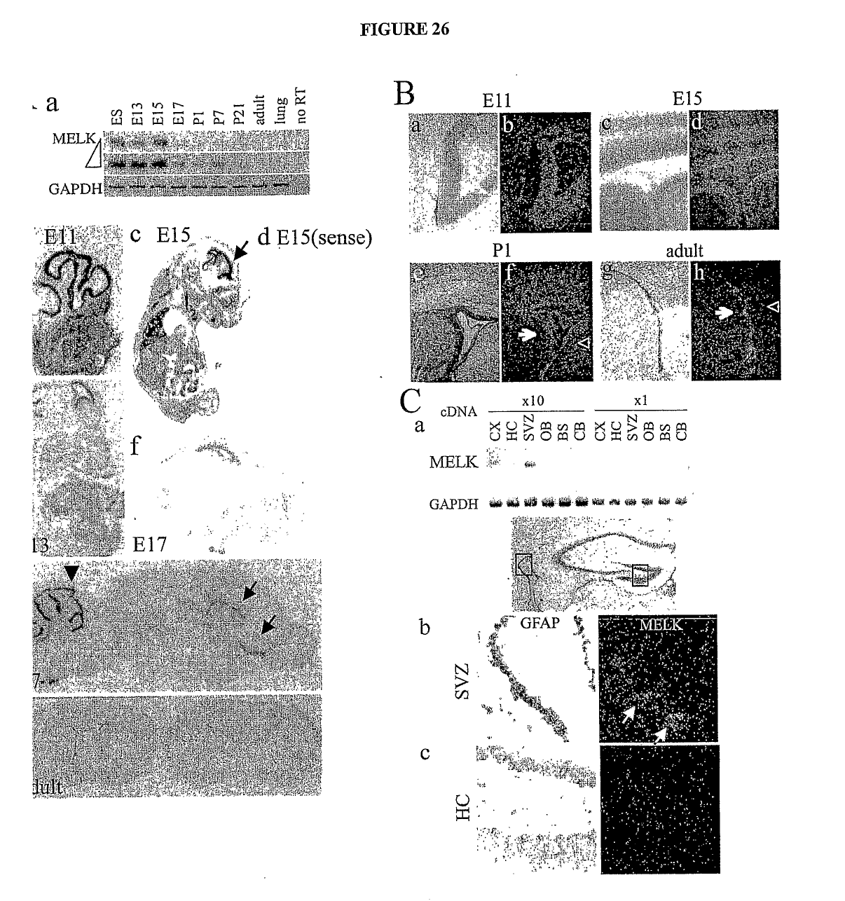

MELK mRNA expression in germinal zones in vivo: Although the above data demonstrate MELK expression in progenitors in vitro, it is critical to establish whether it is also expressed in vivo. Semiquantitative RT-PCR analysis demonstrated that MELK mRNA was expressed in the developing brain during early and mid-embryonic periods with a dramatic decline between E15 and E17 (FIG. 26, panel a). There was no detectable MELK mRNA in the adult brain or lung (used as a control tissue). Expression in embryonic stem (ES) cells was relatively high, similar to that in the earliest embryonic brain (FIG. 26Aa, 1st lane).

Genes associated with MPC should be expressed within the proliferative germinal zones that house these cells. In situ hybridization analysis using [³⁵S]-labeled riboprobes demonstrated that MELK mRNA was expressed throughout central nervous system (CNS) within periventricular germinal zones (G7) as early as E11. This expression persisted through early postnatal periods to adulthood, including cells of the anterior subventricular zone (SVZa) and rostral migratory stream (RMS) (FIG. 26A, panel b to h). No labeling was detected with sense probes (FIG. 2A, panel d), and identical labeling was observed with multiple probes, including ones directed against the entire open reading frame (shown here) or against the original 3′ fragment previously identified using representational difference analysis. No specific hybridization was detectable in the CNS outside of germinal zones, indicating that MELK was not expressed by mature cell types. In the adult brain, the only hybridization was found within cells in the SVZ lining the lateral ventricle (FIG. 26A, panel h), along its entire rostrocaudal extent.

FIG. 26B shows emulsion-dipped sections, demonstrating nearly exclusive expression of MELK in CNS germinal zones at multiple ages. In the adult brain, only a minority of SVZ cells hybridized mRNA, and the signal was limited to the lateral side of the lateral ventricle, suggesting that MELK is expressed by a subset of progenitors in the adult mouse brain (arrows in C). In comparison to the expression in SVZ, no specific hybridization was detected in the adult hippocampus (HC), suggesting that MELK is not expressed in adult hippocampal-derived progenitors. FIG. 26C (photomicrographs) shows in situ hybridization of an adult section counterstained for GFAP immunoreactivity, demonstrating the absence of MELK mRNA in hippocampus and its presence in the SVZ of the same section. Lack of MELK expression in HC, and presence in SVZ, was further confirmed by RT-PCR (FIG. 26C, upper panels).

FIG. 26 illustrates data showing MELK is downregulated during ontogeny, and brain expression is restricted in the neurogenic regions throughout development. FIG. 26A. MELK expression during brain development by RT-PCR and in situ hybridization (ISH). Total RNA was extracted from embryonic stem cells (ES) and whole brains from E13 to adult, and was reverse-transcribed into cDNA. The amount of cDNA from each sample was normalized by examining expression of GAPDH as an internal control. MELK is strongly expressed in ES cells. In the brain, MELK mRNA is detected at the earliest stage examined, with levels peaking at E15 and then rapidly declining from E17 on. ISH with radiolabeled antisense MELK cRNA demonstrates high levels of expression in the neural tube as early as E11, and is present in periventricular germinal zones (GZ) throughout embryonic and early postnatal brain development. Together with GZ, intense MELK signals are also observed throughout the entire rostral migratory pathway (RMS; arrows in c and g), and in the developing cerebellum (CB, arrowhead in g). No signal is identified by radiolabeled sense MELK cRNA (d). FIG. 26B. ISH of SVZ with multiple ages. The right panels at each age are cresyl violet staining. Strong signals are detected in the whole germinal zones in E11 and E15 brains, and in the postnatal brain, MELK signals are stronger in the anteriolateral ventricular wall (arrows) than medial ventricular wall (arrowheads) (f and h). FIG. 26C. RT-PCR with different regions of adult brain shows that MELK expression is detectable in the SVZ but not in the hippocampus (HC) or cerebellum (CB; a). ISH with a brain section, which covers both SVZ and HC, detects MELK positive signals only in SVZ (arrows in b) but not in HC (c). Abbreviations, CX; cerebral cortex, OB; olfactory bulb, BS; brain stem.

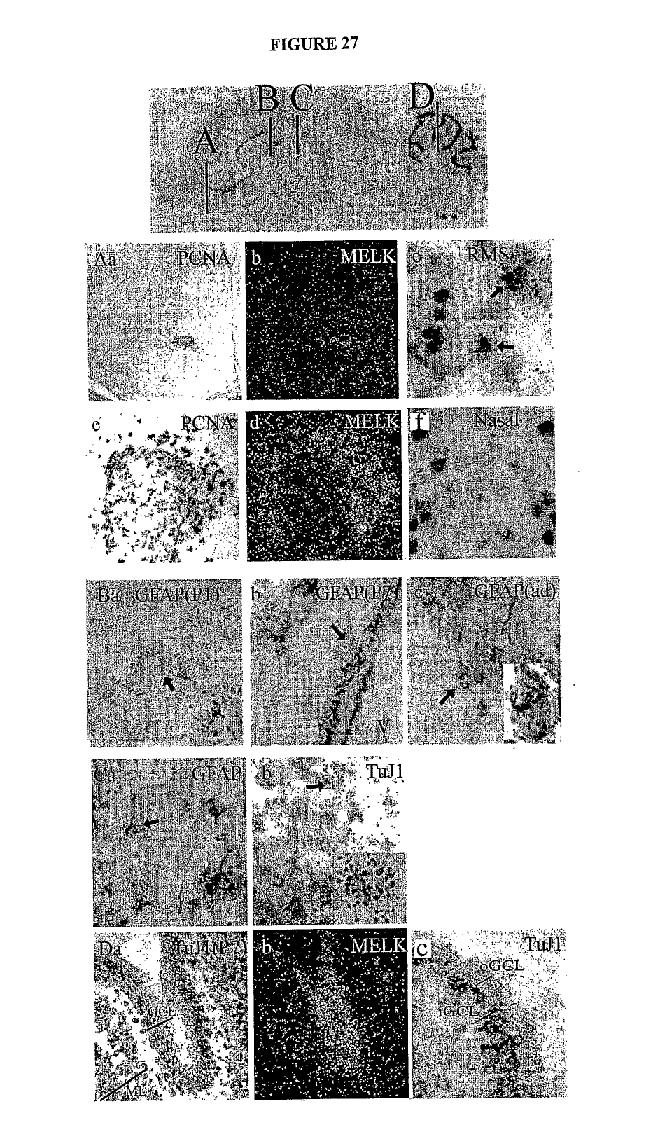

To better define cell types that express MELK, we performed double labeling in situ hybridization/immunohistochemistry analyses (see FIG. 27). MELK labeling occurred in cells expressing the proliferation marker PCNA, as exemplified in the rostral migratory stream in FIG. 27A (a-e)(a). MELK is not a general marker for cell proliferation, however, as it was not expressed by extracerebral PCNA-positive cells in the head (FIG. 27A, panel f).

MELK also exhibited some colocalization with GFAP although the extent of this colocalization was dependent on the developmental stage being analyzed. Throughout embryonic and early postnatal ages (P1), MELK expression was detected in GFAP-negative cells (FIG. 27B, insets in a and b), consistent with the hypothesis that few progenitors express GFAP at these early ages. However, during postnatal development, as GFAP expression increased in SVZ progenitors, MELK mRNA was detected in some GFAP-expressing cells. In the adult SVZ, MELK expression was readily detectable in GFAP-positive cells (inset in FIG. 27B, c). It was not clear whether MELK was readily detectable in GFAP-negative cells in the adult SVZ. Although MELK was not expressed in the adult hippocampus, as described above and shown in FIG. 26C, MELK, was indeed expressed in the hippocampus early postnatal ages, at least GFAP to P7, as shown in FIG. 27C. In the hippocampus at P7, MELK signal was detected in GFAP-positive cells at the hilar border of the dentate gyrus, a site of intense neurogenesis (inset in FIG. 27C, panel a). TuJ1-positive neurons in the dentate gyrus (or, indeed, anywhere else) did not express MELK (inset in FIG. 27C, b).

In addition to expression in the periventricular and hippocampal neurogenic regions, MELK mRNA was also identified within the external granule cell layer of the cerebellum as shown in FIG. 27D. Expression was detectable in the outer, proliferative, EGL with little or no expression in the inner premigratory zone, which is stained by the TuJ1 (βIII tubulin) antibody (FIG. 27D, panel c). Expression in the EGL was detectable as early as the EGL can be distinguished clearly from the rhombic lip at E13, and persisted until postnatal ages. Following the disappearance of the EGL by P14, cerebellum, MELK expression was no longer detectable in the cerebellum.

FIG. 27 illustrates data demonstrating that MELK is expressed only in proliferating PCNA-positive cells, but not in TuJ1-positive neuroblasts in developing brains. Dipped slides after hybridization with MELK cRNA were stained with multiple cell type specific markers. FIG. 27A. Coronal section at the frontal lobe at P7. MELK signals are restricted in the RMS in the cortex (a and b). MELK-positive cells in RMS are largely double-labeled with cell proliferation marker, PCNA (c and d, arrows in e). In contrast with PCNA-positive cells in the brain, MELK is not detected in extracranial PCNA-positive cells (f). FIG. 27B. Majority of MELK-positive cells in the SVZ are not stained with GFAP in the embryonic brains and early postnatal brains (arrows in a and b), while subpopulation of MELK positive cells turned into positive for GFAP in the adult SVZ (arrow in c). FIG. 27C. In the HC at P7, MELK signals are detected in GFAP-positive cells in the hilar border (arrow in a), but not in TuJ1-positive cells in the dentate gyrus (arrow in b). FIG. 27D. In the CB at P7, MELK mRNA was exclusively identified in the granule cell layer (GCL; a and b), particularly in the outer proliferative region, but not in the inner TuJ1-positive migrating neuroblasts (c).

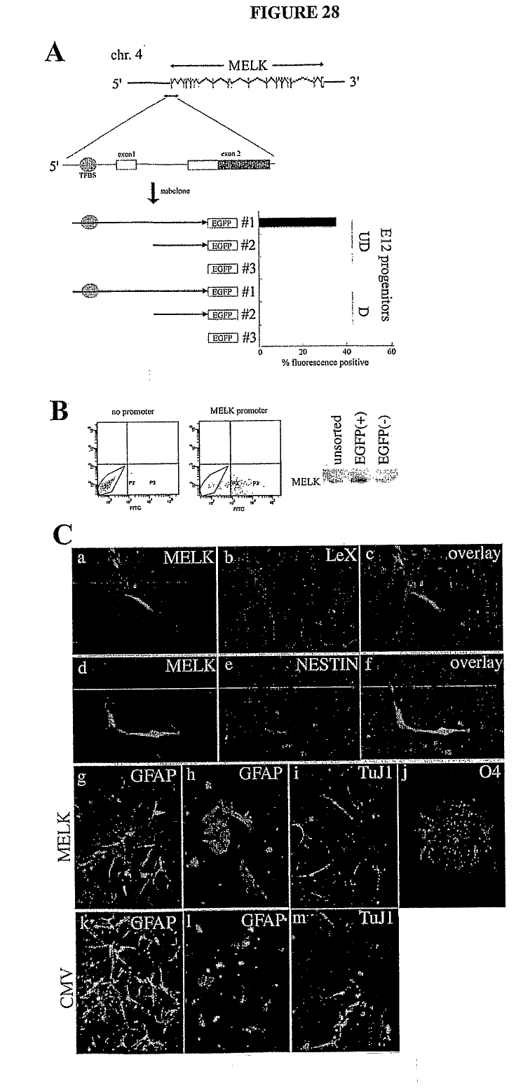

The MELK regulatory element lies upstream of its first exon, and is active only in undifferentiated LeX-positive neural progenitors. Both mouse and human MELK genes have 16 axons with a translation initiation site at exon 2 (see FIG. 28A). The homology of amino acid sequence between these two species is as high as 89%. The mouse gene is located in chromosome 4, and multiple transcription factor binding sequences lies XX kb upstream of mouse exon one (see FIG. 29); and XX kb upstream of human exon one. Therefore, not only the coding region of MELK is highly conserved between these two species, but the 5′-regulatory region in the genome is quite similar in mouse and human, suggesting similar mechanisms of transcriptional regulation.

In order to define the MELK regulatory region in the genome, DNA fragments carrying different lengths of the region upstream of MELK the translation initiation site were subcloned into a promoterless EGFP vector (PMELK-EGFP). Only the larger vector (#1 in FIG. 28A) contained multiple transcription factor binding sequences (TFBS) and the first exon. These TFBS are located at between −2764 bp and −2453 bp from the starting ATG codon, and includes 4 AP2 sites, 8 SpI and 1 NFkB site (see FIG. 29), implying that this region could serve as an active promoter. The three reporter constructs, demonstrated in FIG. 28A, including one without any promoter segment (#3), were transfected into E12 progenitors. A vector using the CMV promoter (PCMV-EGFP) served as a positive control. Following transfection, cells were analyzed by FACS for EGFP expression. As shown in FIG. 28A, fluorescence-positive cells were only found in undifferentiated (LTD) E12 progenitors transfected with a vector containing TFBS (#1). Under this condition, about 27% of cells were categorized as fluorescence-positive. Undifferentiated progenitors transfected with other vectors, or differentiated progenitors transfected with vector #1 contained very few (less than 0.5% of the population) EGFP-expressing cells. The positive control vector with CMV promoter yielded 71.1%, and 69.4% of fluorescence-positive populations in UD progenitors and D cells, respectively. These findings are in agreement with the RT-PCR analyses which demonstrated MELK-expression in undifferentiated, but not differentiated cells, and supports the use of this regulatory element as a functional MELK promoter sequence.

In order to further investigate the specificity of the MELK promoter (PMELK) sequence, cells were transfected with PMELK-EGFP or control vectors and then sorted based on EGFP expression. RT-PCR analysis was used to detect MELK expression both in EGFP positive and in negative populations (see FIG. 28B). As seen in the right panel, the PMELK-EGFP-positive fraction was highly enriched for MELK mRNA as compared to the EGFP-negative fraction or unsorted cells. Since transfection efficiency was not 100%, the faint band observed in the EGFP-negative fraction was expected.

Using the PMELK-EGFP construct we further sought to characterize the cellular specificity of MELK expression in cortical progenitors derived from E12 embryos. Cells were propagated as neurospheres, plated onto substrate, transfected and then stained either prior to or following differentiation (withdrawal of mitogen). Results are shown in FIG. 28C. The morphologies of cells expressing EGFP driven by the CMV promoter were heterogeneous, while MELK promoter-driven EGFP-positive cells were relatively homogeneous with a fusiforme shape (FIG. 28C). Immunocytochemical analysis using confocal fluorescence microscopy was performed on transfected cells using antibodies directed against LeX and nestin (to label progenitors) in proliferating cultures (panels a-f). Differentiated cells were assayed using antibodies to beta tubulin III (Tuji1; neurons), GFAP (astrocytes) and O4 (oligodendrocytes) to label differentiated cells in either proliferating (FIG. 28C: panels g and k) or differentiating (panels h-j, l, m) cultures. MELK-positive fluorescent cells co-localized with LeX and with nestin staining in proliferating cultures (panels a-f), but no PMELK-driven EGFP was detected in cells expressing differentiation markers, even in proliferating cultures (panel g-j). These data indicate that the MELK promoter is active only in LeX/nestin-positive neural progenitors, and not in more differentiated cells. Furthermore, the data are consistent with, and support the findings of native MELK expression in vivo and in vitro described above.

FIG. 28 illustrates data showing that the regulatory element of MELK transcripts is localized in the upstream of its first exon, and is active only in undifferentiated neural progenitors. FIG. 28A. Two genomic fragments with different lengths were isolated from the upstream of the coding region of MELK, and were subcloned into a vector encoding green fluorescence protein sequences (EGFP) without a promoter sequence. Fluorescence-positive populations are compared both in undifferentiated (UD) and differentiated (D) progenitors. Only clone #1 with 3.5 kb genomic fragment encodes multiple transcription factor binding sequences (TFBS) and the first exon of MELK gene. UD progenitors transfected with clone #1 have fluorescence positive populations; however, either UD progenitors transfected with other vectors or D progenitors transfected with clone #1 have no detectable fluorescence positive populations. FIG. 28B. FACS analysis of transfected cells identifies 27.1% EGFP positive cells (P3 quadrant), while 0.5% are positive when transfected with the same plasmid lacking the MELK promoter sequence. After separating fluorescence-positive cells and negative cells by flow cytometry, total RNA was extracted from both populations. RT-PCR demonstrates that the EGFP-positive population, but not negative one, has highly enriched MELK expression. FIG. 28C. The MELK promoter drives EGFP in cells co-expressing neural progenitor-markers, but not differentiated cell type-markers in E12 cortical progenitors. MELK expressing cells, indicated by EGFP expression (panels a and d) co-localizes with the neural progenitor cell markers, LeX (b) and nestin (e), but does not co-localize with GFAP-positive astrocytes (g) in UD progenitors at E12. In D progenitors, EGFP expression is not present, indicating the absence of MELK in differentiated cell types (g-j). In the control cultures, transfected with the PCMV-EGFP construct, all differentiated cell types express EGFP (k-m).

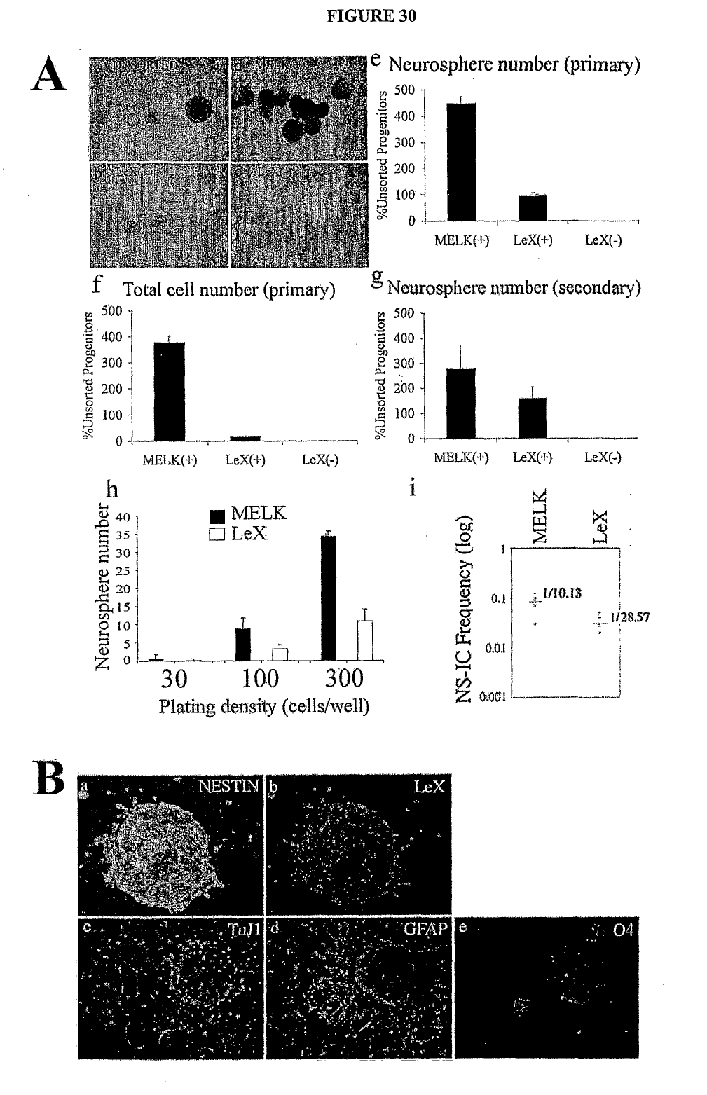

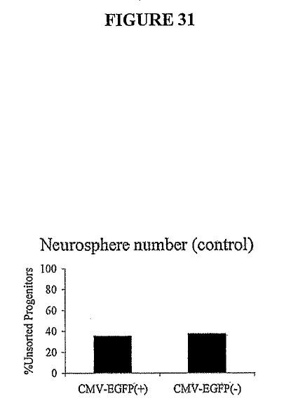

MELK is a marker for tripotent, self-renewing progenitors in embryonic cortical cultures: MPC have the fundamental properties of self-renewal and multipotency. Therefore, we tested the ability of MELK-expressing cells to form primary and secondary neurospheres and examined the differentiation capacity of these spheres. Previous studies have demonstrated that LeX-positive cell fractions are highly enriched in neurosphere-forming cells, and we used this property to compare to the capacity of MELK-expressing cells. Progenitors were cultured as spheres, plated on polyornithine-fibronectin substrate, propagated in bFGF and then were either sorted using anti-LeX antibody or transfected with PMELK-EGFP. After that, these progenitors were sorted for EGFP-positive and negative cells as described in Methods, below. Following sorting, the cells were propagated as “primary” neurospheres (initial spheres derived from adherent progenitors). As demonstrated in FIG. 30A, MELK-positive E15 progenitors generated approximately 5 times more primary neurospheres than LeX-positive cells at a density (2,000 cells/ml) deemed to be clonal or near-clonal. This suggests that the MELK-positive fraction of LeX-positive cells is more highly enriched for sphere-initiating cells. LeX-negative populations did not contain neurospheres when plated at this density. In addition, progenitors formed “secondary” neurospheres (passaged from primary spheres) at this density, indicating that MELK-positive progenitors were capable of self-renewal (FIG. 30A, panel g). Control cultures transfected with PCMV-EGFP yielded equivalent percentages of neurospheres in EGFP positive and negative fractions (see FIG. 31).

In order to more accurately determine the frequency of neurosphere initiating cells (NS-IC), sorted progenitors from E12 cultures were plated at 300, 100 or 30 cells/well in a 96 well plate using serial dilutions. The two lower densities are clearly clonal as our previous experiments demonstrated that virtually all sphere generated from a starting density of 1,000 cells/ml or less are derived from a single cell. At each density, MELK-positive progenitors gave rise to significantly greater numbers of spheres than did LeX positive progenitors. Combining the data from each of the three dilutions, one out of 10.13 MELK-positive progenitors were NS-IC, while 1 out of 28.57 LeX-positive cells were NS-IC (FIG. 30A, panel h and i). Thus, even at extremely low seeding density, MELK-expressing cells were highly enriched for NS-IC.

Neurospheres formed from MELK-expressing cells are derived from multipotent progenitors. Staining of undifferentiated neurospheres revealed that virtually all cells expressed nestin and LeX (FIG. 30B, panel a and b), markers, albeit imperfect, of neural progenitors. After differentiation of primary or secondary spheres, staining revealed that the spheres formed neurons, astrocytes and oligodendrocytes (FIG. 30B, panel c, d, and e). Thus, MELK-expressing stem cells are self-renewing, multipotent progenitors, meeting at least some of the criteria to be called stem cells. Furthermore, the expression of MELK, as indicated by PMELK-driven EGFP fluorescence is highly useful to enrich for MPCs. This, in combination with the expression data in vivo and in vitro, indicates that MELK expression is a useful marker for MPC.

FIG. 30 illustrates data showing that MELK-expressing progenitors are neurosphere-initiating stem cells. FIG. 30A. Neurospheres were grown from MELK-expressing progenitors as well as from LeX-sorted and unsorted cells at low density (2000 cells/mL). Seven days later, the numbers of neurospheres were counted for each condition. Neurospheres are reliably produced under all conditions with the exception of the LeX-negative fraction (FIG. 30A panels a-d). FIG. 30A Panel e shows neurosphere numbers in comparison with unsorted progenitors seeded and propagated following transfection of adherent progenitors, and panel f shows its corresponding cell numbers. FIG. 30A Panel g shows secondary neurosphere numbers after dissociation of the primary spheres counted in panel e in comparison with the primary neurosphere numbers from unsorted progenitors. The graph in FIG. 30A panel h shows the numbers of neurosphere resulting from the seeding of 300, 100 or 30 cells, achieved by serial dilution, of MELK-positive cells and LeX-positive cells. Based on the numbers of resultant neurospheres, the frequency of neurosphere-initiating cells (NS-IC) is analyzed and shown in panel i. FIG. 30B. Neurospheres formed from MELK-expressing cells are derived from typical multipotent stem cells. Secondary neurospheres from MELK positive progenitors were stained as spheres (upper panels) or following differentiation in the absence of mitogen. UD spheres stained with anti-nestin and anti-LeX antibodies. Differentiated spheres demonstrate TuJ1-positive neurons, GFAP-positive astrocytes, and O4-positive oligodendrocytes.

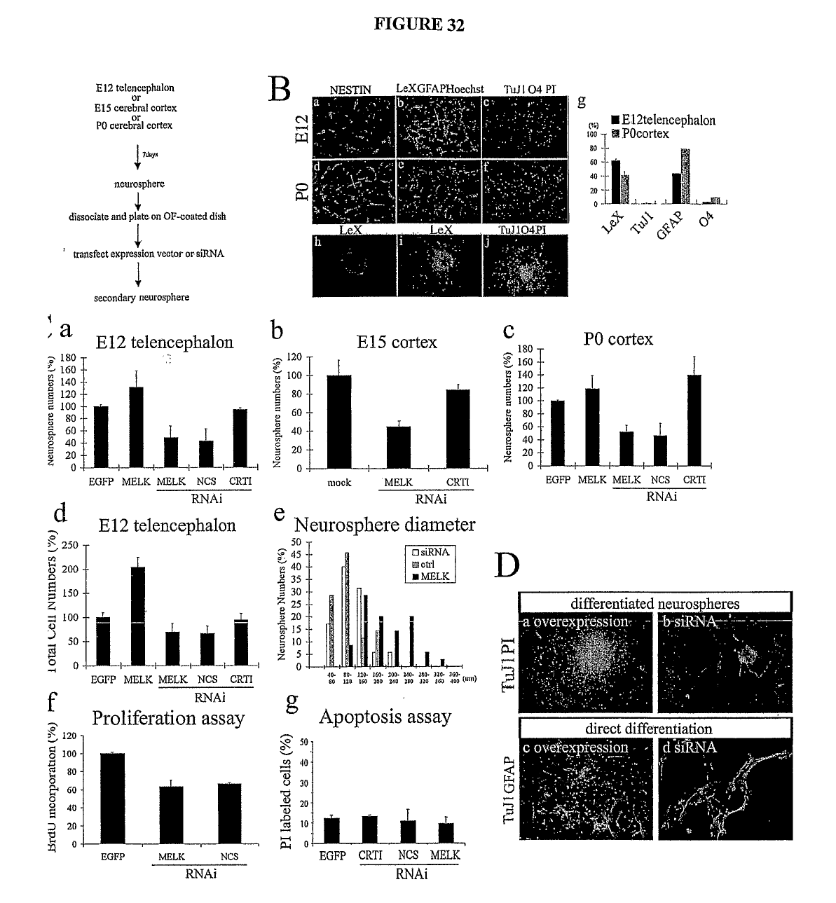

MELK regulates MPC proliferation. The studies described above demonstrate that MELK is expressed by MPC. In order to determine the function of MELK in these cells, we used both “traditional” overexpression experiments as well as knockdown (inhibition) with small interfering RNA (siRNA). FIG. 32A shows the experimental-strategy employed. Neurospheres were generated from the following: E12 telencephalon as a stage of neurogenesis, E15 and P0 cerebral cortex as stages of transition and gliogenesis. After 7 days in culture as spheres, the cells were plated onto polyomithine/fibronectin substrate. The monolayers of progenitors derived from neurospheres were transduced with expression vectors or appropriate double-strand RNA (dsRNA). Using PCMV-EGFP we estimated transfection efficiency at approximately 70%. Forty-eight hours after transfection, cells were collected for RNA extraction to verify the manipulated gene doses.

FIG. 32 illustrates data from experimental manipulation of MELK influences neural progenitor proliferation: MELK-overexpressing progenitors generate more neurospheres, and MELK downregulation diminishes neurosphere numbers. FIG. 32A. Experimental design. Neurospheres were cultured, dissociated, and plated on polyornithine/fibronectin-coated dishes. Adherent progenitors were then transfected grown as secondary neurospheres. FIG. 32B. Characterization of adherent progenitors from neurospheres generated from E12 telencephalon and P0 cerebral cortices (a-f). Monolayer progenitor cultures from neurospheres were immunostained for nestin, LeX, GFAP, TuJ1, and O4 antibodies. PI was used for nuclear staining. The majority of adherent cells from both ages are nestin-positive (a and d), with both LeX positive and GFAP positive subpopulations, (b, u and e, f. The graph in panel g shows that E12 progenitors contain more LeX positive populations, and in turn, P0 progenitors contain more GFAP-positive cells. Three times more O4-positive oligodendrocytes are found in P0 progenitors, whereas no cells are stained with TuJ1 antibody in either condition. After passaging adherent progenitors back into sphere conditions, they form LeX positive secondary neurospheres (h), which are capable of differentiation into all three cell types (j). FIG. 32C. Sphere counts (a-c), total cell counts (d), sphere diameters (e), and percent BrdU incorporation (f), percent apoptotic cells (g) following overexpression or knockdown of MELK in adherent progenitors from E12 telencephalon (a, d-g), E15, and P0 cerebral cortecies (b and c). Overexpression of MELK gives rise to more neurospheres and knockdown of MELK with siRNA reduces the number of neurospheres compared to the control conditions. Knockdown of nucleostemin produced similar results to knockdown of MELK as expected. The graph of total cell numbers of the resultant spheres shows strong effect by overexpression of MELK into E12 progenitors (d). Histograms of the percentage of neurospheres in each size group (e) indicate that the diameters of neurospheres were similar in MELK siRNA and control cultures, while the diameters of MELK-overexpressing spheres are greater. At least 3 independent experiments for each developmental age had been done to confirm the results shown here. The number of proliferating cells (f) and apoptotic cells (g) following MELK or nucleostemin knockdown are measured by incorporation of BrdU antibody or Propidium Iodide and Hoescht, respectively. Proliferation is inhibited by siRNA for MELK and nucleostemin, while the results (+/−SEM) of apoptosis assay demonstrate no significant differences 3 days following treatment. FIG. 32D. Effect of MELK for neural progenitor differentiation. Secondary neurospheres, which are derived from primary neurospheres following transfection, were induced to differentiate (a and b). As demonstrated by TuJ1-staining, the neurogenic ability of sphere-forming cells is not altered by changing MELK expression levels in the adherent progenitors. Dissociated progenitors from the primary E12 neurospheres were transfected with MELK expression vector or siRNA for MELK, and were directly differentiated for 5 days (c and d). Abundant TuJ1-positive neurons are induced in both conditions.

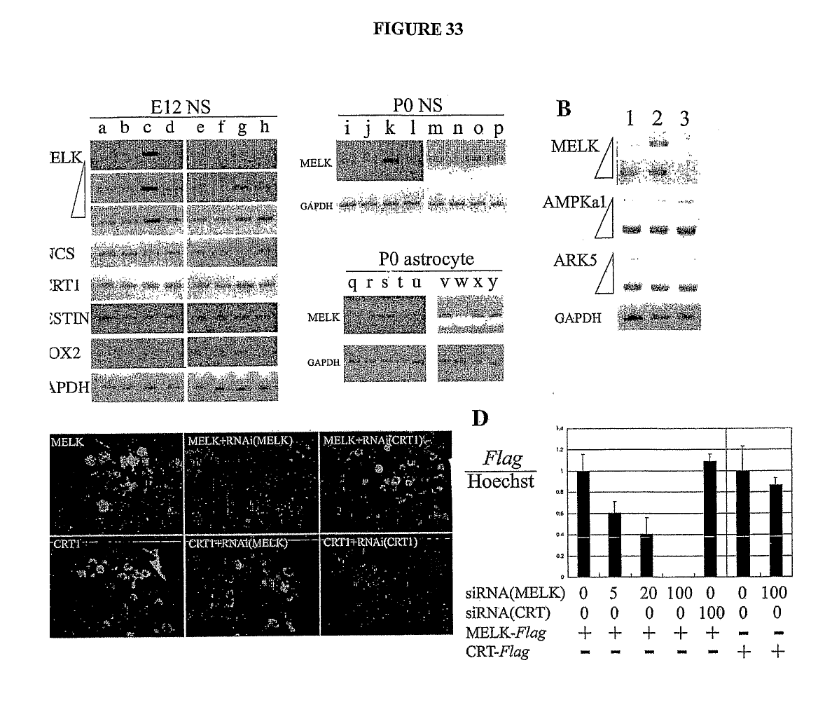

The specificity and efficacy of the overexpression and siRNA vectors used is described below, and illustrated in FIG. 33. FIG. 33A illustrates data showing the expression levels of MELK in E12 progenitor cultures after transduction of various constructs. Serial cycles of RT-PCR for MELK demonstrated that overexpression of MELK, but not of control vectors including pCMV-EGFP, a self-inactivating lentiviral vector (CSCG), or a phosphoserine phosphatase-expression vector, resulted in a specific increase in MELK mRNA.

To evaluate specificity of RNA interference constructs, progenitors were transduced with 2 different concentrations of small interfering RNA (siRNA), 10 and 100 nM. Both concentrations resulted in lower MELK expression levels compared to the controls in which cultures were treated with siRNA for calreticulin (CRT1) or nucleostemin, two genes known to be expressed by neural stem cells. On the other hand treatment with siRNA for nucleostemin or CRT1 resulted in specific knockdown of these genes without interfering with MELK expression. To further exclude nonspecific effects by siRNA, we also examined the effects of MELK siRNA on expression of SOX2 and nestin mRNA levels. Neither was changed by any siRNA treatment used (FIG. 33B).

Additionally, in order to further address the specificity of gene silencing, the effects of MELK siRNA expression, of the nearest AMPK-family members was investigated in the mouse neuroblastoma, Neuro2a (N2a) cells—cells that we have found to express MELK and at least two other family members, AMKKa1 and ARK5. Although MELK expression levels were altered, neither overexpression nor siRNA targeting MELK affected the expression levels of other AMPK-family members, AMPKa1 and ARK5 (FIG. 33B). The other nearest member, AMPKa2, was not detected in any conditions.

To test whether siRNA knockdown selectively influenced expression at the protein level, N2a cells were first transfected with a Flag-tagged MELK construct (MELK-Flag) or a control construct containing the CRT1 coding region tagged Flag (CRT1-Flag), together with either MELK or CRT1 siRNA. As shown in FIG. 33D, treatment with MELK siRNA resulted in specific silencing of Flag expression only in those cells transfected with MELK-flag, while treatment with CRT1 siRNA knocked down flag expression only in cells co-transfected with CRT1-Flag. To quantify the effect of gene silencing, normalized fluorescence intensity was measured by ELISA for flag signal for each condition (FIG. 33D). The results demonstrated a clear, specific, dose-dependent reduction of protein by MELK siRNA.

FIG. 33 illustrates data showing MELK expression is specifically altered by the expression vector and by synthesized dsRNA. FIG. 33A and FIG. 33B. Vector specificity. FIG. 33A. Adherent E12 progenitors were transfected as follows 48 hr prior to RNA collection: a, mock transfection, b, EGFP-containing plasmid, c, MELK-expression vector, d, calreticulin1 expression vector (CRT1) e, MELK dsRNA (10 nM), f, MELK dsRNA (100 nM), g, nucleostemin dsRNA (100 nM), h, Crt1 dsRNA (100 nM). Specific overexpression and knockdown are confirmed for MELK, and knockdown of nucleostemin and CRT1 are also specific for each of their conditions, whereas the expression levels of neural stem cell-related genes, nestin and Sox2, are not affected by any gene transduction at the time points tested. The three panes for MELK (as shown by the triangle) correspond to increasing numbers of PCR cycles. FIG. 33B. Manipulation of MELK mRNA does not influence the levels of other AMPK family members. Semiquantitative RT-PCR shows relative expression levels of MELK, AMPKa1 and ARK5 mRNA in N2a cultures transfected as follows: mock-transfected control (1), MELK-overexpression (2), and MELK dsRNA (3). FIG. 33C and FIG. 33D. MELK siRNA specifically knocks down its protein expression. FIG. 33C. Immunocytochemistry using anti-Flag antibody following transfection of primary progenitors with the MELK-Flag expression vector (a-c) or CRT1-Flag expression vector (d-f). Dual transfection with siRNA for MELK decreased Flag signals only for MELK-Flag vector (b and e), while dual transfection with siRNA for CRT1 decreased Flag signals for CRT1-Flag vector (c and f). FIG. 33D. Fluorescence intensity of Flag was measured for each condition and normalized for cell content by Hoescht nuclear staining. Each intensity (i SEM) is based on three independent experiments and confirms the findings in C.

Given the high degree of specificity of the reagents, as discussed-above, we then examined the influence of MELK overexpression and siRNA vectors on neural progenitors in E12, E15, and P0 according to the scheme outlined in FIG. 6A, similar to that used for analysis of neurosphere formation. Neurospheres were generated from the cortex in the presence of bFGF. After 1 week, the spheres were dissociated and plated onto polyomithine/fibronectin-coated dishes and then transfected with the appropriate vector. In addition to mock transfection, we used nucleostemin and CRT1 siRNAs as controls, due to reports of the effects of these genes on NSC proliferation. These adherent cultures resemble the characteristics of progenitors on each age respectively. E12 telencephalic cells largely contain nestin/LeX positive cells, with a minority of cells that immunostained for GFAP, and virtually no TuJ1 or O4-staining cells (FIG. 32B). P0 cortical cells have fewer LeX-positive progenitors with more GFAP-positive cells, some of which are also LeX-positive. Although E12 cultures do not contain O4-positive cells, 2.4% of the cells in the P0 cortical cultures are oligodendrocytes. Spheres were then generated from these attached cultures after 24 hours. These spheres were propagated for 1 week in bFGF, measured, counted and then replated on poly-L-lysine coated coverslips to assay differentiation potential. To assay potency, we differentiated E12-derived spheres by removal of growth factor and plating on substrate and found that they reliably and readily formed neurons, astrocytes and oligodendrocytes (FIG. 32B, panel j).

Overexpression of MELK in neural progenitors yielded an increased number of spheres and total cells after transfection, while knockdown resulted in a diminished number of spheres compared to controls, indicating that MELK regulates the proliferation of sphere-forming progenitors (FIG. 32C, panel a-c). The total number of cells within cultures was affected, as well, with MELK overexpression resulting in a greater number of cells and knockdown in fewer cells. In addition to a greater numbers of total cells, MELK overexpression resulted in larger spheres, as shown in FIG. 32C, panel e, compared to control conditions or siRNA for MELK. This latter finding suggests that MELK overexpression influences not only sphere-initiating cells, but also cells that contribute to overall sphere size.

The number of neurospheres and total cells can be altered by affecting either cell proliferation or survival. To address this question, proliferation was analyzed by labeling with BrdU and apoptosis was measured by nuclear propidium iodide (PI) uptake and nuclear morphology using Hoechst labeling in progenitors at 48 hours after transfection of RNAi. As shown in panel f and g of FIG. 32C, cell proliferation is inhibited by MELK siRNA, while apoptosis is not significantly affected. These data suggest that MELK influences proliferation itself rather than simply survival of proliferating cells.

Spheres generated by MELK knockdown or overexpression were multipotent, yielding neurons, astrocytes and oligodendrocytes. As shown by staining using the TuJ1 antibody (FIG. 32D, panel a and b), the neurogenic capacity is not significantly altered by the change of MELK expression. These observations indicate that endogenous MELK likely regulates the proliferation of sphere-forming cells, which are, in turn, multipotent without influencing the relative numbers of differentiated cells, i.e., the proliferation of committed progenitors. These experiments, however, do not determine whether regulation of MELK can directly influence differentiation potential of progenitor cells. To assess this possibility, we analyzed the effects of MELK knockdown and overexpression in E12 cortical progenitors that were then directly differentiated for 5 days by withdrawal of bFGF. If MELK were to directly influence differentiation potential or proliferation of a committed progenitor, then one would expect to see different proportions of neurons and glia. MELK-overexpression and MELK-downregulation did not affect the formation of neurons, astrocytes, or oligodendrocytes in these cultures (see FIG. 32D, panel c and d). Taken together, these functional experiments indicate that MELK regulates MPC proliferation without a major effect on the proliferation of committed progenitors or on cell fate decisions.

MELK function is independent of the PTEN controlled signaling pathway and is likely mediated through the proto-oncogene B-Myb. The tumor suppressor PTEN regulates MPC proliferation without influencing cell fate. Since MELK also regulates MPC self-renewal, we sought to determine whether MELK functions via a similar mechanism. PTEN acts by antagonizing P13 kinase activity and indirectly inhibiting the phosphorylation of AKT serine/threonine kinase. In order to determine the relationship between MELK functions and the PTEN-controlled signaling pathway we performed a series of experiments using PTEN conditional knock-out animals and PTEN null neurospheres. First, we documented that MELK expression level as well as pattern are not influenced by PTEN status. FIG. 7Aa shows that MELK is expressed at a similar level in the germinal zones of wildtype (WT) and PTEN conditional knock-out animals in which PTEN was deleted specifically in nestin-expressing cells and their differentiated progenies as previously described in Groszer (2001) Science 294:2186-2189. This result suggests that MELK expression is not controlled by PTEN or PTEN controlled signaling pathway.

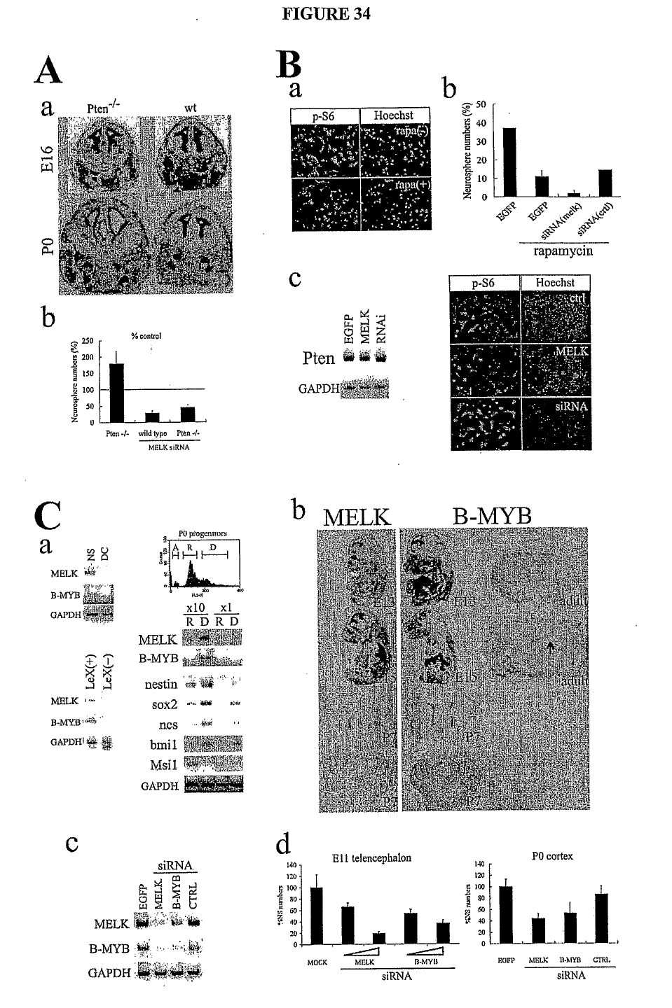

Given that MELK was expressed in PTEN-deficient progenitors, the next set of experiments was designed to assess the effects of MELK knockdown on MPC derived from wild type and Pten conditional knockout mutants. As previously described in Groszer (2001) Science 294:2186-2189, PTEN-deficient embryos yielded greater numbers of neurospheres than wildtype littermates. As is shown in FIG. 34Ab, downregulation of MELK by MELK siRNA significantly and similarly diminished the sphere-forming capacity of both wild type and PTEN-deficient mice. This finding indicates that MELK functions in the absence of PTEN and that PTEN is not likely to function via a MELK-dependent mechanism. Furthermore, MELK overexpression or knockdown did not influence the level of PTEN mRNA as determined by semiquantitative RT-PCR (FIG. 34B, panel c), indicating that MELK does not function by inhibiting PTEN expression.

To further explore a possible relationship between MELK and the AKT pathway, we examined MELK function in rapamycin-treated cultures. Rapamycin is a specific inhibitor of mTOR (REF), a kinase which is regulated by P13 kinase/AKT and, in turn, phosphorylates the S6 kinase. E14 wildtype progenitors were treated with rapamycin (as described in Methods). The efficacy of this treatment was demonstrated by reduced phospho-S6 staining in treated cultures (FIG. 34B, panel b). As would be predicted if AKT action were a determinant of NS formation, rapamycin treatment diminished the numbers of neurospheres formed. The number of neurospheres was further diminished by the addition of MELK siRNA to the rapamycin treatment (FIG. 34B, panel b). These findings demonstrate that MELK function is not dependent on the actions of mTOR. MELK does not appear to function upstream of the mTOR pathway, either, since overexpression or knockdown of MELK did not influence the extent of phospho-S6 as indicated by immunostaining (FIG. 34B, panel c). Taken together, this series of experiments indicates that MELK functions parallel to the PTEN/AKT/mTOR pathway.

Recently, using a lung cancer cell line, see Seong (2002) Biochem. J. 361:597-604, we demonstrated that MELK binds the transcription factor Zpr9, which, itself, has been shown bind to the proto-oncogene, B-myb, a cell cycle regulator (see, e.g., Seong (2003) J. Biol. Chem. 278:9655-9662). In order to test the hypothesis that MELK function is mediated by B-myb, we first examined B-myb expression in our culture systems (FIG. 34C, panel a-c). As is the case for MELK, B-myb was highly enriched in NS from P0 cortex compared to DC, and among cells in NS, B-myb was also enriched in the LeX positive fractions (FIG. 34C, panel a). When proliferating P0 progenitors were divided into apoptotic, resting (G0/G1 phase), and dividing (G2/M and S phases) populations by propidium iodide (PI) labeling followed by FACS, both MELK and B-myb were only found in dividing populations (FIG. 34C, panel b). These data are consistent with previous findings demonstrating that B-myb was highly upregulated at late G1 and S phase. Interestingly, most NSC-related genes were also enriched in dividing population, with the only exception being Msi-1, which is known to be expressed in some lineage-committed astrocytes. Thus, MELK and B-myb were expressed in similar populations of neural cells.

If MELK acts via B-myb, then the inhibition of MELK should diminish B-myb expression. As is shown in FIG. 34C, panel c, MELK siRNA downregulates (inhibits) both MELK and B-myb. In turn, B-myb siRNA treatment results in a near complete loss of B-Myb mRNA, similar to its effects on MELK mRNA. B-myb siRNA yielded a slight decrease of MELK expression. Such might be predicted if the number of MELK-expressing cells were reduced by B-myb treatment, even at the early timepoints used to assay mRNA. Control siRNA did not influence either MELK or B-MYB expression. We next determined if knockdown of B-Myb produced similar effects to MELK, as would be predicted if MELK were upstream of B-myb. As shown in FIG. 34C, panel d, knockdown of B-Myb resulted in a dose-dependent decrease in neurosphere formation from progenitors. Thus, these data suggest that inhibition of endogenous MELK expression downregulates B-myb, which, in turn, results in the reduction of neurosphere numbers.

FIG. 34 illustrates data demonstrating that the signaling pathway of MELK is independent of Pten-akt pathway, and is likely through a protooncogene, B-myb. FIG. 34Aa. In situ hybridization of MELK using Pten conditional mutant. Pten mutant mice have a phenotype of enlarged brains as well as hydrocephalus at P0. MELK expression at the germinal zones, however, is not altered by Pten deletion in the brains both at E16 and at P0. FIG. 34Ab. MELK function was analyzed in Pten-deleted neural progenitors. The graph shows the ratio of neurosphere formation in each condition compared to the wild type. FIG. 34B. Decreased neurosphere formation by mTOR specific antagonist, rapamycin, does not alter the effect of MELK siRNA. XX nM of rapamycin was added in the culture and 48 hours later, treated progenitors as well as untreated progenitors, were stained with phospho-S6 antibody (a). The graph in FIG. 34 panel b shows the effect of MELK siRNA against neurosphere formation from rapamycin treated progenitors. Panel c shows that MELK siRNA treatment does not affect the expression of Pten or phospho-S6. Expression of Pten was compared by RT-PCR between MELK overexpressing progenitors, MELK siRNA treated progenitors, and the control progenitors. In the right panel, neural progenitors were stained with phospho-S6 antibody after treatment with MELK expression vector, MELK siRNA or control vector. FIG. 34C. B-myb studies. RT-PCR of MELK and B-myb using P0 neurospheres (NS), differentiated neurospheres (DC), and E12 neurospheres sorted with LeX antibody (a). Panel b shows RT-PCR with MELK, B-myb, and other genes after separation of P0 progenitors into apoptotic (A), resting (R), and dividing (D) populations. The upper panel shows the flow cytometry of P0 neurospheres using Propidium Iodide. Both MELK and B-myb, as well as some of neural stem cell-related genes, are highly enriched in D cells in neurospheres. In contrast, Msi1 is highly enriched in R cells. RT-PCR in panel c shows the expression of MELK and B-myb after treatment of neural progenitors with siRNA for either MELK, B-myb, or the control gene. Panel b shows in situ expression of B-myb using brains at multiple developmental ages. MELK expression at each corresponding stage is shown in parallel. Panel c is RT-PCR of both MELK and B-myb after treatment of siRNA against MELK or B-myb. B-myb expression is inhibited by siRNA treatment for both MELK and B-myb. Panel d. Functional study of B-myb using neural progenitors in E11 telencephalon and P0 cortex. E11 progenitors were treated with MELK siRNA at 25 nM and 100 nM, or B-myb siRNA at 25 nM and 100 nM. P0 progenitors were also treated with 100 nM of siRNA targeting each gene. The graph shows the ratio of neurosphere formation from each progenitors compared to the control condition. Each data shown here had been confirmed by at least three independent experiments.

MELK regulates the transition from GFAP expressing progenitors to a proliferative,

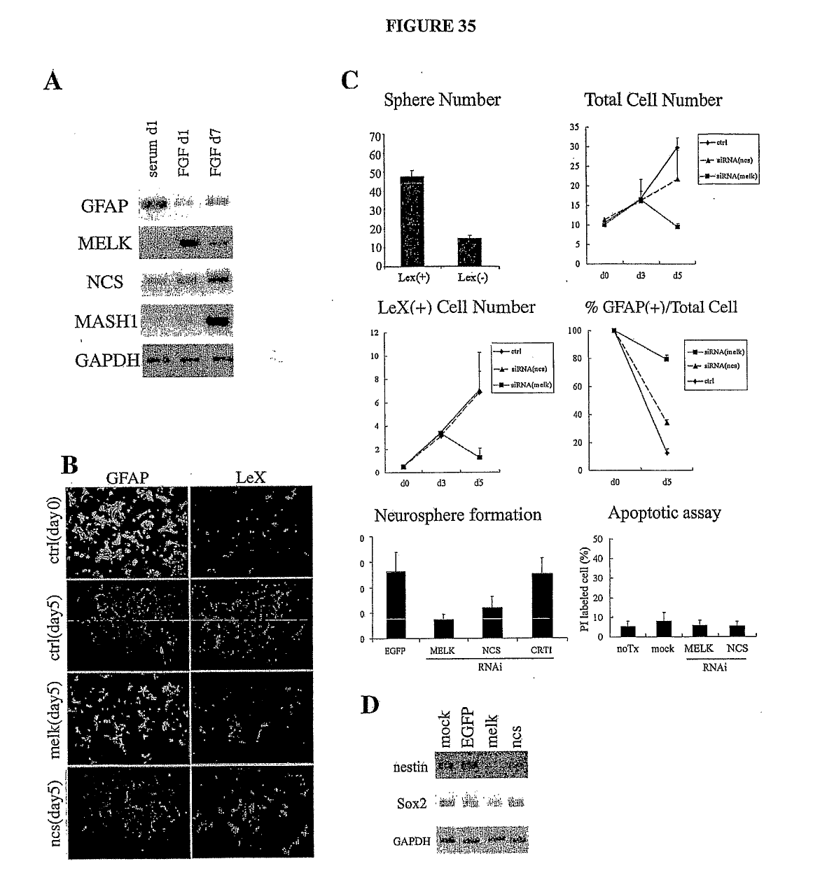

multipotent state: Recent studies have documented the ability of subventricular zone (SVZ) GFAP-positive astrocytes to form rapidly amplifying GFAP-negative progenitors in the presence of bFGF (see, e.g., Tropepe (2001) Dev. Biol. 208:166-188). These transition processes can be monitored by RT-PCR and immunocytochemistry, see FIG. 35A and FIG. 35B). Twenty-four hours of bFGF-treatment resulted in diminished GFAP mRNA expression and increased nucleostemin expression. MASH1 mRNA was upregulated following 7 days, but not 24 hr of treatment (FIG. 35A). On day 0 virtually all the cells in culture were GFAP immunoreactive, while very few were LeX positive (FIG. 35B and FIG. 35C). Five days after placement in bFGF, GFAP immunoreactivity had dramatically declined, and approximately 30% of the total cell numbers were LeX positive (FIG. 35B and FIG. 35C). The LeX positive cells were either GFAP negative or weakly GFAP positive.

The LeX-positive cells among GFAP-positive astrocytes function as progenitors. Astrocyte cultures were exposed to bFGF for two days and then subjected to FACS using anti-LeX antibody. The number of neurospheres produced from the LeX positive fraction was markedly higher than the number from the LeX negative fraction (FIG. 35C) and the LeX positive cell-derived spheres were competent to produce neurons. Taken together, these findings are consistent with the hypothesis that the addition of bFGF to astrocyte cultures results in the conversion of GFAP positive/LeX negative astrocyte-like progenitors to rapidly proliferative, GFAP negative LeX positive MPC. Furthermore, this culture system is reminiscent of the in vivo transition from “type B”, astrocyte-like stem cells to “type C” rapidly proliferative multipotent progenitors (see, e.g., Alvarez-Buylla (2002) Brain Res. Bull. 57:751-758).

MELK mRNA expression was examined during these transition states. Strikingly, as shown in FIG. 35A, MELK expression was upregulated as these GFAP-positive cells were stimulated with bFGF. These observations suggest that high levels of MELK expression is either a reflection of the MPC state or that MELK regulates this process. In order to determine whether this transition was dependent on MELK, we knocked down MELK expression during bFGF stimulation by treating with dsRNA just prior to addition of bFGF. Strikingly, siRNA for MELK, but not for nucleostemin, resulted in diminished numbers of neurospheres and prevented the appearance of LeX positive cells (FIG. 35B and FIG. 35C). Instead, there was a relative persistence of GFAP-positive cells. Knockdown of MELK also resulted in the reduced expression of nestin and SOX2 during bFGF treatment.

One potential explanation for the effects of MELK siRNA on proliferation in these cultures is that MELK mediates the survival of proliferating cells. In order to test this possibility, we counted the number of condensed or fragmented nuclei in treated cultures 3 days after treatment with MELK or control siRNAs and found no significant differences. Taken together, these findings show that MELK promotes the transition of SVZ astrocytes into proliferating progenitors without a major effect on cell survival.

FIG. 35 illustrates data showing that MELK upregulation is necessary for transition from GFAP-positive neural stem cells into GFAP-negative, LeX positive rapidly amplifying progenitors in vitro. FIG. 35A. MELK expression during transition of GFAP-positive cells with bFGF. MELK is upregulated as positive cells were stimulated to form rapidly amplifying LeX-positive progenitors with bFGF as indicated by RT-PCR analysis. Analysis of marker genes confirmed the change of gene expression corresponding to neural stem cells (GFAP), progenitors (NCS), and neuroblasts (MASH1). MELK upregulation was identified earlier than that of other marker genes. FIG. 35B and FIG. 35C. MELK siRNA prevents the proliferation of LeX-positive cells in astrocyte cultures. Cultures containing GrAP-positive cells were transfected with siRNA for MELK. Mock transfection and siRNA treatment for nucleostemin were used as control. FIG. 35B. Cells were stimulated with bFGF to form multipotent progenitors and stained with GFAP and LeX. Note the decreased LeX staining only in cultures treated with MELK siRNA. FIG. 35C. LeX-positive cells derived from GFAP-positive astrocytes form neurospheres at a higher frequency than LeX negative cells (mean+/−SEM). Counts demonstrate that MELK siRNA blocks the increase in the total number of cells and LeX positive cells following bFGF treatment for the number of days indicated, and also prevents the decline in the number of GFAP positive cells normally seen with bFGF treatment. No increase in the number of apoptotic cells is observed in MELK or nucleostemin siRNA-treated cultures. The counts of stained cells are based on two independent experiments for each condition. FIG. 35D. RT-PCR analysis of cultures, demonstrating that MELK siRNA results in lower levels off nestin and SOX2 mRNA than controls following bFGF treatment.

In these studies we demonstrated that MELK is expressed by and is a marker for self renewing, tripotent progenitors-MPC and that MELK regulates MPC proliferation, based on in vivo expression and in vitro functional studies. We had previously identified MELK in a genome-wide screen for neural stem/progenitor genes, using the strategy of genetic subtraction coupled to cDNA microarrays and downstream in situ hybridization. MELK was found to be highly expressed in multiple populations of neurospheres as well as hematopoietic stem cells and enriched in CNS germinal zones, making it a strong candidate to regulate neural stem cell functional processes.

MELK is a useful marker for multipotent neural progenitors in the embryonic brain. In these studies we demonstrated that MELK expression can be used to prospectively isolate MPC from developing brain. The MELK promoter element drives EGFP expression faithfully, allowing for isolation of MELK-expressing cells by FACS. This approach has been taken using other genes, including nestin, Msi1, and SOX2. Using the nestin promoter/enhancer or the Msi1 promoter, these approaches others have found that approximately 1-2% of the isolated, EGFP-expressing cells form neurospheres. Other, non gene-based methods have also been used to enrich for neural stem cells from brain or neurospheres, including size, and exclusion of Hoescht dye. Using this latter method, Kim (2003) J. Neuroscience 23:10703-10709, reported that approximately 10% of the side population formed multipotent neurospheres when sorted from other neurospheres. Positive sorting using anti-LeX antibody has also been shown to enrich for neural stem cells in adult brain. In these studies, we demonstrated that the relative enrichment for neurosphere initiation with PMELK-EGFP was greater than that for LeX, as well as for previously reported results using other promoters. There was approximately the same level of enrichment reported using side population purification. The cell-sorting and immunocytochemical data presented here are consistent with the hypothesis that MELK-expressing cells are the subset of LeX-positive cells that form neurospheres.

MELK regulates MPC proliferation in vitro. Data described herein demonstrated that MELK regulates MPC proliferation from embryonic and early postnatal neocortex. Cell cycle regulation has not been reported previously as a function either for MELK or for other members of the AMPK/snf1 family, which largely mediate cell survival under hostile conditions.

According to current theory, any division by a stem cell should be self-renewing, with some divisions being symmetric, resulting in two stem cells, and others being asymmetric, resulting in one stem cell and another committed cell. The neurosphere formation assay has been used previously to demonstrate that Bm1, a transcriptional repressor regulates neural stem cell self-renewal, as well as the transcription factor SOX2 and the phosphatase Pten. MELK regulates symmetric MPC self-renewal in our assays, since in the studies described herein we showed diminished numbers of secondary multipotent neurospheres in siRNA-treated cultures. It is not yet clear if MELK is also capable of regulating asymmetric divisions.

Interestingly, we did not see a significant effect of MELK siRNA on neurosphere size. Neurosphere size is determined by the symmetric and asymmetric proliferation of MPC cells as well as the proliferation of more committed progenitors and the size and packing density of the cells within the spheres. The lack of effect of MELK siRNA on neurosphere size may be because those MPC that do form spheres are ones that escaped transfection with dsRNA, because the knockdown of MELK in neural progenitors by the siRNA is temporary or because MELK does not regulate the proliferation of more committed progenitors within the spheres, which may make up the bulk of the sphere volume.

MELK overexpression, as expected, results in greater numbers of neurospheres. This is compatible with MELK regulating MPC self-renewal. However, overexpression also regulates the size and number of cells per neurospheres. One explanation for this is that the effects of the expression vector persist in the cultures during neurosphere enlargement, and that MELK continues to act upon MPC proliferation. Alternatively, it is possible that the forced, ectopic expression of MELK in other progenitors also promotes their proliferation, resulting in larger neurospheres. The latter possibility would suggest that more cells are capable of responding to MELK than normally express it.

It is not known whether MELK only regulates symmetric self-renewing division, rather than simply regulating any proliferation by a MPC. It has been proposed that stem cell genetic programs exist for the purpose of self-renewing division-sets of genes that would distinguish stem cells from other proliferating progenitors that do not self-renew. One gene proposed to play such a role is Bmi-1, a polycomb transcriptional repressor. Bmi-1 null neural stem cells (MPC) have diminished proliferative/self-renewal capacity, whereas there is no influence on more restricted progenitors. Like Bmi-1, MELK is expressed in several self-renewing stem cell populations, including embryonic (shown here), hematopoietic, and neural stem (MPC) cells (as shown in the data presented herein). Also, like Bmi-1, MELK is required for neural stem cell self-renewal, at least in vitro. Since our functional studies are focused only on the MPC derived from embryonic and neonatal brains and not on other cell types, it is still yet to be determined if MELK actually mediates proliferation in a cell that is constrained to self-renewing divisions in other regions. The data presented herein—the expression data—clearly indicated, however, that MELK is not likely to be a general cell cycle gene, as it is not expressed by PCNA positive cells in the head outside the brain.

Does MELK regulate the transition of “type B” cells into “type C” cells? Little is known about the mechanisms underlying how GFAP-expressing, slowly proliferative or quiescent type B cells transition to more highly proliferative self-renewing type C cells. MELK expression is upregulated as GFAP-positive cells derived from the postnatal cortex are driven to a neurogenic state with bFGF. Previous studies demonstrate that GFAP-expressing cells derived from the neocortex-presumably the subventricular zone-form clonal neurospheres and produce neurons in the presence of bFGF. The studies described herein found that approximately 5% of GFAP-expressing cells in culture express LeX without bFGF stimulation, and that the number of LeX-expressing cells increases up to 30% following bFGF treatment. These LeX-expressing cells form multipotent neurospheres. These changes are accompanied by an upregulation of MELK as well as a downregulation of GFAP. There is a later upregulation of NCS and MASH1, consistent with the hypothesis that the addition of bFGF results in the development of a multipotent and then neurogenic state. Interestingly, cultures have a fairly high level of expression of both SOX2 and nestin prior to bFGF addition, suggesting that the cells have immature, progenitor-like features prior to the addition of bFGF and are not “fully differentiated” astrocytes.

Addition of MELK siRNA inhibits the transition of GFAP-positive cells to GFAP-negative, LeX-positive progenitor cells in the presence of bFGF without a dramatic influence on the GFAP-positive populations. MELK siRNA also inhibited NS formation from GFAP-positive cells by bFGF treatment. The studies described herein indicate that, in vitro, MELK regulates this transition.

Potential mechanism underlying MELK function. One of the key regulators of MPC self-renewal is Pten which, in turn, is a part of the Akt/mTOR/S6 Kinase pathway. Our data are not consistent with MELK exerting its function through manipulation of this pathway, nor are they consistent with the actions of the pathway being dependent on MELK function. Instead, the studies described herein suggest that MELK function is mediated by the proto-oncogene B-Myb. This transcription factor is known to promote G1 to S transition. The studies described herein demonstrated that MELK knockdown (inhibition of expression) strongly downregulates B-myb expression in primary progenitors, and B-myb knockdown also inhibits NSC proliferation in a dose dependent manner. Technical limitations—the presence of a significant fraction of untransfected cells and difficulty in dual transfection with siRNA—have prevented the definitive demonstration that MELK acts through B-myb (however, the invention is not limited by any particular mechanism of action).

The studies described herein demonstrated that MELK is a gene highly expressed in the proliferating progenitors in vivo and regulates MPC proliferation in vitro. These findings demonstrate that the compositions and methods of the invention are effective in the treatment, diagnosis and prognosis of brain pathological states, such as brain tumors, where aberrant progenitor proliferation is implicated. These findings demonstrate that the compositions and methods of the invention can be used to study brain development and CNS repair mechanisms and treatments.

Materials and Methods

Neural progenitor cultures. Neurosphere cultures were prepared as described previously. Cortical telencephalon was removed from E12 CD-1 mice, and cerebral cortex was isolated from E15 and P0 (Charles River). Cells were dissociated with a fire-polished glass pipette, and resuspended at 50,000 cells per ml in DMEM/F12 medium (Invitrogen) supplemented with B27 (Gibco BRL), 20 ng/ml basic fibroblast growth factor (bFGF) (Peprotech), and penicillin/streptomycin (Gemini Bioproducts) and heparin (Sigma). Growth factors were added every 3 days. For differentiation, culture medium was replaced into Neurobasal (Invitrogen) supplemented with B27 without FGF onto poly-L-lysine (PLL)-coated dishes, and maintained up to 5 days. For secondary sphere formation assay, the primary spheres were dissociated and plated into 96-well microwell plates in 0.2 ml volume of growth media including conditioned media at 40,000 cells per milliliter, and the resultant sphere numbers were counted at 7 days.

To assay the influence of gene knockdown or overexpression, the neurosphere culture system was modified. Neurospheres were propagated for 1 week and then dissociated with trypsin (0.05%) followed by trituration with a fire-polished pipette. The cells were then placed in DMEM/F12 with 2% fetal bovine serum (Gibco BRL #26140-079, Carlsbad) and plated onto polyornithine/fibronectin coated glass coverslips (Sun et al., 2001). After 6 hours, the serum-containing medium was removed and the cells were placed back in the neurosphere growth medium without heparin and supplemented with bFGF (20 ng/ml). Transfection was then performed as described below. To assay the sphere-forming potential of the transfected cells, they were lifted off the plate with trypsin (0.05%) and then placed into Neurobasal media supplemented with B27, bFGF and heparin (see, e.g., Wachs (2003) Lab Invest. 83:949-962). To assay the function of cells expressing EGFP driven by the MELK promoter, 1 week neurospheres were plated onto coverslips as above and transfected. Some cultures were then placed into neurosphere conditions to assay sphere-forming potential, while others were propagated and differentiated on the coated coverslips after transfection. Proliferation activity was measured by BrdU incorporation for 24 hours at DIV3, which is shown as O.D. 492 nm, using Cell Proliferation ELISA, BrdU (colorimetric) kit (Roche), according to manufacturer's protocol.

GFAP-positive astrocyte-enriched cultures. Primary astrocyte cultures were prepared from P1 mouse cortices as described previously (see, e.g., Imura, T., et al., J. Neurosci. (2003) 23:2824-2832). Briefly, as cells became confluent (12-14 DIV), they were shaken at 200 rpm overnight to remove nonadherent cells and obtain pure astrocytes, and passaged on PLL-coated coverslips for RNA collection or FGF stimulation. To determine the expression and function of MELK during the production of neural stem cells from astrocyte-like progenitors, the media were changed to neurosphere growth medium with heparin.

Quantitative and Semiquantitative RT-PCR. Total RNA was isolated from each sample using TRIzol (GIBCO BRL), and 1 ug of RNA was converted to cDNA by reverse transcriptase following the manufacturer's protocol (Impron). For semiquantitative RT-PCR, the amount of cDNA was examined by RT-PCR using primers for glyceraldehyde-3-phosphate-dehydrogenase gene (GAPDH) as an internal-control from 20 to 45 cycles. After correction for the amount of GAPDH amplicons for each set, the resultant cDNA was subjected to PCR analysis using gene-specific primers listed in FIG. 15A. The protocol for the thermal cycler was: denaturation at 94° C. for 3 min, followed by corresponding cycles of 94° C. (30 sec), 60° C. (1 min), and 72° C. (1 min), with the reaction terminated by a final 10 min incubation at 72 C. Control experiments were done either without reverse transcriptase and/or without template cDNA to ensure that the results were not due to amplifications of genomic or contaminating DNA. Each reaction were visualized after 2% agarose gel electrophoresis for 30 min, and the expression levels were compared between the cDNA samples on a same gel.

For quantitative RT-PCR. DNase treated RNA samples (1 ug) were directly reverse transcribed with IMPROMT-II (ImPromt-II) RT™ (Promega). Real-time PCR was performed utilizing a LightCycler rapid thermal cycler system (Roche Diagnostics) according to the manufacturer's instructions. A master mix of the following reaction components was prepared to the indicated end—concentrations: 8.6 μl of water; 4 μl of Betaine (1M) 2.4 μl of MgCl₂ (4 mM), 1 μl of primer nix (0.5 μM) and 2 μl LightCycler (Fast Start DNA Master SYBR Green I: Roche Diagnostics). LightCycler MASTERMIX™ (18 μl) was filled in the LightCycler glass capillaries and 2 μl cDNA was added as PCR template. A typical experimental run protocol consisted of an initial denaturation program (95° C. for 10 min), amplification and quantification program repeated 45 times (95° C. for 15 s, 62° C. for 5s, 72° C. for 15 s followed by a single fluorescence measurement). Relative quantification is determined using the LightCycler Relative Quantification Software (Roche Diagnostics), which takes the crossing points (CP) for each target transcript and divides them by the reference GAPDH CP.

Immunocytochemistry. Immunocytochemistry of neurospheres, adherent progenitors, and neonatal astrocytes were performed as described previously (See, e.g., Geschwind (2001) Neuron 29:325-339). Cells were fixed with 3% paraformaldehyde (PFA) for 30 minutes and immunostained with the following primary antibodies: nestin (Rat401; 1:200; Developmental Studies Hybridoma Bank), LeX (CD15; 1:200; Invitrogen), TuJ1 (1:500, Berkely Antibodies), GFAP (1:1000, DAKO), and O4 (1:50, Chemicon). Primary antibodies were visualized with Alexa 568 (red), 488 (green) and 350 (blue) conjugated secondary antibodies (Molecular Probes). Hoechst 333342 (blue) and PI (red) were used as a fluorescent nuclear counterstain.

Sphere Diameter Analysis. Secondary neurospheres from E12.5 telenceophalon were plated into coverslips and fixed with 4% PFA. Diameters of 30-120 randomly chosen spheres from each condition were measured using the Microcomputer Imaging Device Program (MCID). A minimum cutoff of 40 um was used in defining a neurosphere.

Construction of Vectors.

pCMV-MELK. The full-length coding region of mouse MELK was amplified by PCR using mouse embryonic neurospheres as a template, and subcloned into TEASY™ (TEasy) vector (Promega). After sequence verification, MELK fragment was subcloned into pCMV-tag vector (Stratagene) at NotI site.

PMELK-EGFP. The putative MELK promoter region was defined using PromoterScan (Center for Information Technology, National Institutes of Health, Bethesda, Md.). This program indicated that the 2.7 kb upstream of the starting ATG codon had multiple transcription factor binding sequences as is shown in supplemental Table 2. A bacterial artificial chromosome (BAC) clone was obtained from BAC/PAC resources (Children's Hospital Oakland Research Institute in Oakland). Using this BAC clone as a template, 3.5 kb and 0.7 kb upstream of the starting ATG codon of mouse MELK was amplified and subcloned into Teasy vector. After the sequence confirmation, a genomic region of MELK promoter was fused to EGFP polyA (Clontech) yielding PMELK-EGFP. siRNA Synthesis. siRNA was synthesized using the Silencer siRNA Construction Kit following manufacturer's instruction (Ambion). Four different targeting sequences were designed from coding region of mouse MELK. Each of the four demonstrated different levels of mRNA knockdown, and one was chosen for further analysis. Its targeting sequences are as follows: MELK specific siRNA, AACCCAAGGCTCAACAAGGAdTdT (SEQ ID NO:4).

Flow Cytometry and Sorting. Flow cytometry and sorting of EGFP+ cells from E12- and E15-derived neural progenitors were performed an a FACS Vantage (Becton-Dickinson) using a purification-mode algorithm. Gating parameters were set by side and forward scatter to eliminate dead and aggregated cells, and EGPF vector without promoter transfected cells were used for a negative control to set the background fluorescence; false positive cells were less than 0.5%. For isolation of LeX+ cells, E12 progenitors were labeled with LeX antibody (Invitrogen) for 30 minutes and ALEXA 530™ was used for flow cytometry and sorting. Background signals were investigated by the same set of progenitors without primary antibody.

Transient Transfection. Cells were transfected using LIPOFECTAMINE 2000™ (Invitrogen) following manufacturer's protocol. For transfection of plasmid vectors, the cells were incubated with reagents for 6 hours with the primary progenitor cells, and for 24 hours with N2a cells. For transfection of the double stranded siRNA complex, serial doses of siRNA from 5 to 200 nM were tested to obtain specific knockdown of the gene of interest, and 100 nM was chosen as the concentration for functional study. Incubation with siRNA complex was 6 hours with primary cells and 24 hours with cell lines.

A number of aspects of the invention have been described. Nevertheless, it will be understood that various modifications may be made without departing from the spirit and scope of the invention. Accordingly, other aspects are within the scope of the following claims.

What is claimed is:

1. A composition for inhibiting the growth, proliferation, differentiation and/or survival of a neural stem cell or a cancer cell, or progenitor stem cell thereof, comprising at least one compound capable of
(i) inhibiting transcription of a gene or inhibiting translation of a gene's transcript, wherein the gene is selected from the group consisting of a maternal embryonic leucine zipper kinase (MELK) gene, a T-LAK cell-originated protein kinase (TOPK) gene, a phosphoserine phosphatase (PSP) gene, a forkhead box M1 (FoxM1) gene, a B-myb gene, a Rho/Rac/Cdc42-like GTPase activating protein (RACGAP) gene, a kinesin superfamily protein member 4 (KIF4) or KIF4A gene, a cell cycle control protein CDC2 gene, a EZHa gene, a HCAP-G gene, a minichromosome maintenance (MCM)-7 (MCM7) gene, a chromatin assembly factor-1A (CHAF-1A) gene, a minichromosome maintenance protein 6-(MCM6) gene, a thymopoietin (TMPO) gene, a sperm associated antigen 5 (SPAG5) gene, a baculoviral IAP repeat-containing 5 (BIRC5) gene, a thymidylate synthase (TYMS) gene, a karyopherin (importin) alpha 2 (KPNA2) gene, a kinesin family member 2C (KIF2c) gene, a MAD2 (mitotic arrest deficient, homolog)-like 1 (MAD2L1) gene, a NIMA (never in mitosis gene a)-related kinase 2 (NEK2) gene, a BUB1 budding uninhibited by benzimidazoles 1 homolog beta (yeast) (BUB1B) gene, an epithelial cell transforming sequence 2 oncogene (ECT2) gene, a ubiquitin-conjugating enzyme E2C (UBE2C) gene, a fatty acid elongase (FEN1) gene, a H2A histone family, member X (H2AFX) gene, a serine/threonine kinase 6 (STK6) gene, a methyltransferase TI (DNMT1) gene, a proliferating cell nuclear antigen (PCNA) gene, a polymerase A (POLA) gene, a thyroid hormone receptor interactor 13 (TRIP13) gene, a MK167 (proliferation-related Ki-67 antigen) gene and a solute carrier family 35, member B1 (SLC35B1) gene; or
(ii) inhibiting the expression or activity of protein selected from the group consisting of a maternal embryonic leucine zipper kinase (MELK), a T-LAK cell-originated protein kinase (TOPK), a phosphoserine phosphatase (PSP), a forkhead box M1 (FoxM1) protein, a B-myb protein, a Rho/Rac/Cdc42-like GTPase activating protein (RACGAP), a kinesin superfamily protein member 4 (KIF4) or KIF4A protein, a cell cycle control protein CDC2, a EZHa protein, a HCAP-G protein, a MCM7 protein, a CHAF1A protein, a MCM6 protein, a TMPO protein, a SPAG5 protein, a BIRC5 protein, a TYMS protein, a KPNA2 protein, a KIF2c protein, a MAD2L1 protein, a NEK2 protein, a BUB1B protein, a ECT2 protein, a UBE2C protein, a FEN1 protein, a H2AFX protein, a STK6 protein, a DNMT1 protein, a PCNA protein, a POLA protein, a TRIP13 protein, a MK167 (proliferation-related Ki-67 antigen) protein and a solute carrier family 35 (SLC35B1) protein.

2. A pharmaceutical composition comprising at least one composition as set forth in claim 1, and a pharmaceutically acceptable excipient.

3. The composition of claim 1 or the pharmaceutical composition of claim 2, wherein the pharmaceutical composition comprises at least two, three, four or five compounds capable of inhibiting the growth, proliferation, differentiation and/or survival of a neural stem cell or a cancer cell.

4. The composition of claim 1 or the pharmaceutical composition of claim 2, wherein the at least one compound inhibits the growth, proliferation, differentiation and/or survival of a brain tumor cell or a stem cell progenitor thereof.

5. The composition of claim 1 or the pharmaceutical composition of claim 2, wherein the at least one compound inhibits growth, proliferation, differentiation and/or survival of a granule cell precursor cell or a self-renewing neural cancer cell or a stem cell progenitor thereof.

6. The composition of claim 1 or the pharmaceutical composition of claim 2, wherein the compound comprises a nucleic acid, a carbohydrate, a fat, a small molecule or a polypeptide or peptide.

7. The composition of claim 6, wherein the at least one nucleic acid compound capable of inhibiting transcription of a gene or inhibiting translation of a gene's transcript nucleic acid comprises an oligonucleotide.

8. The composition of claim 6, wherein optionally the oligonucleotide comprises an antisense oligonucleotide, a double-stranded inhibitory RNA (RNAi) molecule, a ribozyme, an RNase III-prepared short interfering RNA (esiRNA) or a vector-derived short hairpin RNAs (shRNA).

9. The composition of claim 7, wherein the double-stranded inhibitory RNA (RNAi) molecule, ribozyme, RNase III-prepared short interfering RNA (esiRNA) or vector-derived short hairpin RNAs (shRNA) comprises a subsequence of a promoter or a message of a maternal embryonic leucine zipper kinase (MELK) gene, a T-LAK cell-originated protein kinase (TOPK) gene, a phosphoserine phosphatase (PSP) gene, a forkhead box M1 (FoxM1) gene, a B-myb gene, a Rho/Rac/Cdc42-like GTPase activating protein (RACGAP) gene, a kinesin superfamily protein member 4 (KIF4) or KIF4A gene, a cell cycle control protein CDC2 gene, a EZHa gene, a HCAP-G gene, a MCM7 gene, a CHAF1A gene, a MCM6 gene, a TMPO gene, a SPAG5 gene, a BIRC5 gene, a TYMS gene, a KPNA2 gene, a KIF2c gene, a MAD2L1 gene, a NEK2 gene, a BUB1B gene, a ECT2 gene, a UBE2C gene, a FEN1 gene, a H2AFX gene, a STK6 gene, a DNMT1 gene, a PCNA gene, a POLA gene, a TRIP13 gene, a MK167 (proliferation-related Ki-67 antigen) gene or a solute carrier family 35 (SLC35B1) gene.

10. The composition of claim 6, wherein the at least one polypeptide or peptide compound capable of inhibiting transcription of a gene or inhibiting translation of a gene's transcript nucleic acid comprises (a) an antibody, or (b) a polypeptide or peptide capable of binding a transcriptional activator of a maternal embryonic leucine zipper kinase (MELK) gene, a T-LAK cell-originated protein kinase (TOPK) gene, a phosphoserine phosphatase (PSP) gene, a forkhead box M1 (FoxM1) gene, a B-myb gene, a Rho/Rac/Cdc42-like GTPase activating protein (RACGAP) gene, a kinesin superfamily protein member 4 (KIF4) or KIF4A gene, a cell cycle control protein CDC2 gene, a EZHa gene, a HCAP-G gene, a MCM7 gene, a CHAF1A gene, a MCM6 gene, a TMPO gene, a SPAG5 gene, a BIRC5 gene, a TYMS gene, a KPNA2 gene, a KIF2c gene, a MAD2L1 gene, a NEK2 gene, a BUB1B gene, a ECT2 gene, a UBE2C gene, a FEN1 gene, a H2AFX gene, a STK6 gene, a DNMT1 gene, a PCNA gene, a POLA gene, a TRIP13 gene, a MK167 (proliferation-related Ki-67 antigen) gene or a solute carrier family 35 (SLC35B1) gene.

11. A method for inhibiting the growth, proliferation, differentiation and/or survival of a neural stem cell or a cancer cell or progenitor stem cell thereof, comprising the steps of contacting the cell with a composition as set forth in claim 1.

12. The method of claim 11, wherein the neural stem cell or a cancer cell is a neural tumor cell proliferation or a progenitor thereof.

13. A method for inhibiting the growth, proliferation, differentiation and/or survival of a neural stem cell or a cancer cell, or progenitor stem cell thereof, in an individual in need thereof, comprising the steps of administering to the individual a therapeutically effective amount of a pharmaceutical composition as set forth in claim 2.

14. An array comprising
(a) at least one nucleic acid comprising a gene sequence or a transcript or cDNA sequence, wherein the sequence comprises a maternal embryonic leucine zipper kinase (MELK) sequence, a T-LAK cell-originated protein kinase (TOPK) sequence, a phosphoserine phosphatase (PSP) sequence, a forkhead box M1 (FoxM1) sequence, a B-myb sequence, a Rho/Rac/Cdc42-like GTPase activating protein (RACGAP) sequence, a kinesin superfamily protein member 4 (KIF4) or KIF4A sequence, a cell cycle control protein CDC2 sequence, a EZHa sequence, a HCAP-G sequence, a MCM7 sequence, a CHAF1A sequence, a MCM6 sequence, a TMPO sequence, a SPAG5 sequence, a BIRC5 sequence, a TYMS sequence, a KPNA2 sequence, a KIF2c sequence, a MAD2L1 sequence, a NEK2 sequence, a BUB1B sequence, a ECT2 sequence, a UBE2C sequence, a FEN1 sequence, a H2AFX sequence, a STK6 sequence, a DNMT1 sequence, a PCNA sequence, a POLA sequence, a TRIP13 sequence, a MK167 (proliferation-related Ki-67 antigen) sequence or a solute carrier family 35 (SLC35B1) sequence, or a combination thereof, or
(b) at least one protein or peptide comprising a sequence or subsequence of a protein or peptide comprising a maternal embryonic leucine zipper kinase (MELK) protein, a T-LAK cell-originated protein kinase (TOPK), a phosphoserine phosphatase (PSP), a forkhead box M1 (FoxM1) protein, a B-myb protein, a Rho/Rac/Cdc42-like GTPase activating protein (RACGAP), a kinesin superfamily protein member 4 (KIF4) or KIF4A protein, a cell cycle control protein CDC2, a EZHa protein, a HCAP-G protein, a MCM7 protein, a CHAF1A protein, a MCM6 protein, a TMPO protein, a SPAG5 protein, a BIRC5 protein, a TYMS protein, a KPNA2 protein, a KIF2c protein, a MAD2L1 protein, a NEK2 protein, a BUB1B protein, a ECT2 protein, a UBE2C protein, a FEN1 protein, a H2AFX protein, a STK6 protein, a DNMT1 protein, a PCNA protein, a POLA protein, a TRIP13 protein, a MK167 (proliferation-related Ki-67 antigen) protein or a solute carrier family 35 (SLC35B1) protein, or a combination thereof.

15. A compilation of probes comprising
(a) at least two nucleic acids comprising a gene sequence or a transcript or cDNA sequence, wherein the sequence comprises a maternal embryonic leucine zipper kinase (MELK) sequence, a T-LAK cell-originated protein kinase (TOPK) sequence, a phosphoserine phosphatase (PSP) sequence, a forkhead box M1 (FoxM1) sequence, a B-myb sequence, a Rho/Rac/Cdc42-like GTPase activating protein (RACGAP) sequence, a kinesin superfamily protein member 4 (KIF4) or KIF4A sequence, a cell cycle control protein CDC2 sequence, a EZHa sequence, a HCAP-G sequence, a MCM7 sequence, a CHAF1A sequence, a MCM6 sequence, a TMPO sequence, a SPAG5 sequence, a BIRC5 sequence, a TYMS sequence, a KPNA2 sequence, a KIF2c sequence, a MAD2L1 sequence, a NEK2 sequence, a BUB1B sequence, a ECT2 sequence, a UBE2C sequence, a FEN1 sequence, a H2AFX sequence, a STK6 sequence, a DNMT1 sequence, a PCNA sequence, a POLA sequence, a TRIP13 sequence, a MK167 (proliferation-related Ki-67 antigen) sequence or a solute carrier family 35 (SLC35B1) sequence, or a combination thereof; or
(b) at least two proteins or peptides capable of binding specifically to a protein comprising a sequence or subsequence of a maternal embryonic leucine zipper kinase (MELK) protein, a T-LAK cell-originated protein kinase (TOPK), a phosphoserine phosphatase (PSP), a forkhead box M1 (FoxM1) protein, a B-myb protein, a Rho/Rac/Cdc42-like GTPase activating protein (RACGAP), a kinesin superfamily protein member 4 (KIF4) or KIF4A protein, a cell cycle control protein CDC2, a EZHa protein, a HCAP-G protein, a MCM7 protein, a CHAF1A protein, a MCM6 protein, a TMPO protein, a SPAG5 protein, a BIRC5 protein, a TYMS protein, a KPNA2 protein, a KIF2c protein, a MAD2L1 protein, a NEK2 protein, a BUB1B protein, a ECT2 protein, a UBE2C protein, a FEN1 protein, a H2AFX protein, a STK6 protein, a DNMT1 protein, a PCNA protein, a POLA protein, a TRIP13 protein, a MK167 (proliferation-related Ki-67 antigen) protein or a solute carrier family 35 (SLC35B1) protein, or a combination thereof.

16. A method of identifying a compound that inhibits the growth, growth, proliferation, differentiation or survival differentiation or survival of a neural stem cell or a cancer or tumor cell, or a progenitor stem cell thereof, comprising
(a) providing a candidate compound and a neural stem cell, a cancer or tumor cell, or a progenitor stem cell thereof;
(b) contacting the cell with a candidate compound;
(c) measuring the level of expression of at least one of a maternal embryonic leucine zipper kinase (MELK) gene, a T-LAK cell-originated protein kinase (TOPK) gene, a phosphoserine phosphatase (PSP) gene, a forkhead box M1 (FoxM1) gene, a B-myb gene, a Rho/Rac/Cdc42-like GTPase activating protein (RACGAP) gene, a kinesin superfamily protein member 4 (KIF4) or KIF4A gene, a cell cycle control protein CDC2 gene, a EZHa gene, a HCAP-G gene, a MCM7 gene, a CHAF1A gene, a MCM6 gene, a TMPO gene, a SPAG5 gene, a BIRC5 gene, a TYMS gene, a KPNA2 gene, a KIF2c gene, a MAD2L1 gene, a NEK2 gene, a BUB1B gene, a ECT2 gene, a UBE2C gene, a FEN1 gene, a H2AFX gene, a STK6 gene, a DNMT1 gene, a PCNA gene, a POLA gene, a TRIP13 gene, a MK167 (proliferation-related Ki-67 antigen) gene or a SLC35B1 gene, or a combination thereof, wherein the level of expression of the gene is measured by determining the level of expression of the gene, a message transcribed by the gene or a protein encoded by the gene; and
(d) comparing under substantially the same conditions the level of expression of at least one of the gene, message or protein in a cell not contacted by the candidate compound to the level of expression of at least one of the gene, message or protein in a cell contacted by the compound, whereby the candidate compound is identified as a compound that inhibits the growth, proliferation, differentiation or survival of the cell growth as one that decreases expression the gene, message or protein.

17. The method of claim 16, further comprising assessing the inhibition of growth, proliferation, differentiation, survival and/or self-renewal potential of the cell in the presence of the compound.

18. The method of claim 17, wherein the growth, proliferation, differentiation and/or survival inhibition is assessed by primary sphere formation assay, proliferation or differentiation potential.

19. The method of claim 5, wherein the compound is identified as an inhibitor of growth or proliferation when proliferation or growth of the cell in the presence of the compound is 10%, 20%, 30%, 40%, 50%, 55%, 60%, 65%, 70%, 75%, 80%, 85%, 90% or more inhibited in the presence of the compound.

20. A method of identifying a candidate compound for inhibiting growth or proliferation of a neural stem cell or a cancer or tumor cell, or a progenitor stem cell thereof, comprising
(a) providing a candidate compound;
(b) contacting the candidate compound with a protein comprising a sequence or subsequence of a maternal embryonic leucine zipper kinase (MELK) protein, a T-LAK cell-originated protein kinase (TOPK), a phosphoserine phosphatase (PSP), a forkhead box M1 (FoxM1) protein, a B-myb protein, a Rho/Rac/Cdc42-like GTPase activating protein (RACGAP), a kinesin superfamily protein member 4 (KIF4) or KIF4A protein, a cell cycle control protein CDC2, a EZHa protein, a HCAP-G protein, a MCM7 protein, a CHAF1A protein, a MCM6 protein, a TMPO protein, a SPAG5 protein, a BIRC5 protein, a TYMS protein, a KPNA2 protein, a KIF2c protein, a MAD2L1 protein, a NEK2 protein, a BUB1B protein, a ECT2 protein, a UBE2C protein, a FEN1 protein, a H2AFX protein, a STK6 protein, a DNMT1 protein, a PCNA protein, a POLA protein, a TRIP13 protein, a MK167 (proliferation-related Ki-67 antigen) protein or a solute carrier family 35 (SLC35B1) protein, or a combination thereof; and
(c) measuring or determining the effect of the compound on the biological activity of the protein, whereby a compound that inhibits at least one biological activity of at least one protein is identified as a candidate compound for inhibiting growth or proliferation of a neural stem cell or a cancer or tumor cell, or a progenitor stem cell thereof.

21. The method of claim 20, wherein inhibition of at least one biological activity of at least one protein identifies the compound as a candidate compound for inhibiting the growth, proliferation, differentiation and/or survival of a granule cell precursor cell or a self-renewing neural cancer cell or a stem cell progenitor thereof.

22. A method of diagnosing the metastatic potential of a neural tumor comprising determining the presence or absence of expression of a maternal embryonic leucine zipper kinase (MELK) protein, a T-LAK cell-originated protein kinase (TOPK), a phosphoserine phosphatase (PSP), a forkhead box M1 (FoxM1) protein, a B-myb protein, a Rho/Rac/Cdc42-like GTPase activating protein (RACGAP), a kinesin superfamily protein member 4 (KIF4) or KIF4A protein, a cell cycle control protein CDC2, a EZHa protein, a HCAP-G protein, a MCM7 protein, a CHAF1A protein, a MCM6 protein, a TMPO protein, a SPAG5 protein, a BIRC5 protein, a TYMS protein, a KPNA2 protein, a KIF2c protein, a MAD2L1 protein, a NEK2 protein, a BUB1B protein, a ECT2 protein, a UBE2C protein, a FEN1 protein, a H2AFX protein, a STK6 protein, a DNMT1 protein, a PCNA protein, a POLA protein, a TRIP13 protein, a MK167 (proliferation-related Ki-67 antigen) protein or a solute carrier family 35 (SLC35B1) protein, or a combination thereof.

23. A kit comprising a composition as set forth in claim 1, wherein optionally the kit comprises instructions for practicing the methods of any of claims 11 to 13 or claims 16 to 23.


SEQUENCE LISTING (32 sequences)

SEQ ID NO: 1 - DNA, 19 bp, Artificial Sequence
  mouse MELK
aacccaagggcaacaagga

SEQ ID NO: 2 - protein, 12 aa, Artificial Sequence
  synthetic peptide
NFKTPSKLSEKC

SEQ ID NO: 3 - protein, 17 aa, Artificial Sequence
  synthetic peptide
CTNEDPKDRPSAAHIVE

SEQ ID NO: 4 - DNA, 20 bp, Artificial Sequence
  mouse MELK
aacccaaggctcaacaagga

SEQ ID NO: 5 - protein, 651 aa, Artificial Sequence
  synthetic construct
MKDYDELLKYYELHETIGTGGFAKVKLACHILTGEMVAIKIMDKNTLGSDLPRIKTEIEALKNLRHQHICQLYHVLETANKIFMVLEYCPGGELFDYIISQDRLSEEETRVVFRQIVSAVAYVHSQGYAHRDLKPENLLFDEYHKLKLIDFGLCAKPKGNKDYHLQTCCGSLAYAAPELIQGKSYLGSEADVWSMGILLYVLMCGFLPFDDDNVMALYKKIMRGKYDVPKWLSPSSILLLQQMLQVDPKKRISMKNLLNHPWIMQDYNYPVEWQSKNPFIHLDDDCVTELSVHHRNNRQTMEDLISLWQYDHLTATYLLLLAKKARGKPVRLRLSSFSCGQASATPFTDIKSNNWSLEDVTASDKNYVAGLIDYDWCEDDLSTGAATPRTSQFTKYWTESNGVESKSLTPALCRTPANKLKNKENVYTPKSAVKNEEYFMFPEPKTPVNKNQHKREILTTPNRYTTPSKARNQCLKETPIKIPVNSTGTDKLMTGVISPERRCRSVELDLNQAHMEETPKRKGAKVFGSLERGLDKVITVLTRSKRKGSARDGPRRLKLHYNVTTTRLVNPDQLLNEIMSILPKKHVDFVQKGYTLKCQTQSDFGKVTMQFELEVCQLQKPDVVGIRRQRLKGDAWVYKRLVEDILSSCKV

SEQ ID NO: 6 - DNA, 20 bp, Artificial Sequence
  nestin s primer
ggagcgcgtggcacacatgg

SEQ ID NO: 7 - DNA, 25 bp, Artificial Sequence
  nestin as primer
gtagaccctgcttctcctgctccag

SEQ ID NO: 8 - DNA, 24 bp, Artificial Sequence
  nucleostemins primer
caaatgtggggaaaagcagtgtca

SEQ ID NO: 9 - DNA, 23 bp, Artificial Sequence
  nucleosteminas primer
gcagggggatggcaatagtaacc

SEQ ID NO: 10 - DNA, 20 bp, Artificial Sequence
  Msi1 s primer
gagactgacgcgccccagcc

SEQ ID NO: 11 - DNA, 22 bp, Artificial Sequence
  Msi1 as primer
cgcctggtccatgaaagtgacg

SEQ ID NO: 12 - DNA, 24 bp, Artificial Sequence
  CALR s primer
atatcgactgtgggggcggctacg

SEQ ID NO: 13 - DNA, 27 bp, Artificial Sequence
  CALR as primer
ggtggcagaaagtcccaatcatcctcc

SEQ ID NO: 14 - DNA, 24 bp, Artificial Sequence
  b-myb s primer
cgcctgtcaaaaccctccccttct

SEQ ID NO: 15 - DNA, 24 bp, Artificial Sequence
  b-myb as primer
ataggggcaggggttggaatgtgg

SEQ ID NO: 16 - DNA, 22 bp, Artificial Sequence
  Mash1 s primer
cctcagggggcggtcacaagtc

SEQ ID NO: 17 - DNA, 23 bp, Artificial Sequence
  Mash1 as primer
caccttgctcatcttcttgttgg

SEQ ID NO: 18 - DNA, 24 bp, Artificial Sequence
  NFH s primer
ccacggaaaagcccaaggactcta

SEQ ID NO: 19 - DNA, 21 bp, Artificial Sequence
  NFH as primer
gggggctggctttctttctgg

SEQ ID NO: 20 - DNA, 26 bp, Artificial Sequence
  Melk s primer
cttggatcagaggcagatgtttggag

SEQ ID NO: 21 - DNA, 23 bp, Artificial Sequence
  Melk as primer
gctgtaatcttgcatgacccagg

SEQ ID NO: 22 - DNA, 25 bp, Artificial Sequence
  GFAP s primer
gcagagatgatggagctcaatgacc

SEQ ID NO: 23 - DNA, 18 bp, Artificial Sequence
  GFAP as primer
acctccagccgggcactg

SEQ ID NO: 24 - DNA, 24 bp, Artificial Sequence
  ARK5 s primer
ggtgggtgaactggggctacaaga

SEQ ID NO: 25 - DNA, 20 bp, Artificial Sequence
  ARK5 as primer
tggggagggggatggcagtc

SEQ ID NO: 26 - DNA, 19 bp, Artificial Sequence
  AMPKa1 s primer
cccggcacaccctggatga

SEQ ID NO: 27 - DNA, 21 bp, Artificial Sequence
  AMPKa1 as primer
agggaggagtcgagggaggtg

SEQ ID NO: 28 - DNA, 28 bp, Artificial Sequence
  AMPKa2 s primer
agtgactggcaactatgtgaaaatgaag

SEQ ID NO: 29 - DNA, 27 bp, Artificial Sequence
  AMPKa2 as primer
ttgcagagacgtaataccgagtgagat

SEQ ID NO: 30 - DNA, 21 bp, Artificial Sequence
  siRNA(crtI)  primer
aaagagcagttcttggacgga

SEQ ID NO: 31 - DNA, 21 bp, Artificial Sequence
  siRNA(melk)  primer
aagagagaaatacccgcctca

SEQ ID NO: 32 - DNA, 21 bp, Artificial Sequence
  siRNA(NCS)  primer
aagaactaaaacagcagcaga